(19) United States
(12) Patent Application Publication
(10) Pub. No.: US 2015/0353986 A1
(43) Pub. Date: Dec. 10, 2015

(54) METHODS FOR TREATMENT OF CORONARY HEART DISEASE EVENTS BASED ON LIPOPROTEIN-ASSOCIATED PHOSPHOLIPASE A2 ACTIVITY

(71) Applicants: Patrick Alfred ARENSDORF, Palo Alto, CA (US); Mary Jane CERELLI, Burlingame, CA (US); Robert William GERWIEN, JR., Newington, CT (US); Emi ZYCHLINSKY, Palo Alto, CA (US)

(72) Inventors: Patrick Alfred ARENSDORF, Palo Alto, CA (US); Mary Jane CERELLI, Burlingame, CA (US); Robert William GERWIEN, JR., Newington, CT (US); Emi ZYCHLINSKY, Palo Alto, CA (US)

(21) Appl. No.: 14/540,749

(22) Filed: Nov. 13, 2014

Related U.S. Application Data
(60) Provisional application No. 62/010,403, filed on Jun. 10, 2014.

(51) Int. Cl. C12Q 1/44; A61K 45/06; A61K 38/51; A61K 31/216; A61K 31/573; A61K 38/48; A61K 31/616; A61K 31/352

(52) U.S. Cl. CPC C12Q 1/44; A61K 31/616; A61K 45/06; A61K 31/352; A61K 31/216; A61K 31/573; A61K 38/484; A61K 38/51

(57) ABSTRACT

Methods of treating coronary heart disease (CHD) events by measuring Lp-PLA2 activity are described herein. Described herein are methods that compare Lp-PLA2 activity levels to a binary cut point to guide clinical diagnosis and treatment of CHD. The methods described herein provide robust treatment of patients regardless of race or gender, and may allow the simplification of complex treatment decisions and enhance patient care.

### CROSS REFERENCE TO RELATED APPLICATIONS

This patent application claims priority to U.S. Provisional Patent Application No. 62/010,403, titled “APPARATUS AND METHODS FOR DIAGNOSIS, TREATMENT AND ASSESSMENT OF CORONARY HEART DISEASE EVENTS BASED ON LIPOPROTEIN-ASSOCIATED PHOSPHOLIPASE A2 ACTIVITY,” filed on Jun. 10, 2014 which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are methods, and assays (e.g., compositions, kits, etc.) for of using lipoprotein-associated phospholipase A2 (Lp-PLA₂) activity levels for identifying and treating cardiovascular disease, including diagnosing and prognosticating cardiovascular disease, and for treating coronary heart disease.

### BACKGROUND

Lipoprotein-associated phospholipase A₂ (Lp-PLA₂ or Lp-PLA2), also known as platelet activating factor acetylhydrolase (PAF-AH), is a key participant in the chronic vascular inflammation which characterizes atherosclerosis progression and coronary heart disease. Lp-PLA₂ is a secreted approximately 50 kD phospholipase enzyme encoded by the PLA2G7 gene in humans. It is associated primarily with low-density lipoprotein (LDL) in plasma (Stafforini et al. 1987). Lp-PLA2 has been previously identified and characterized in the literature by Tew et al. (1996) Arterioscler. Thromb. Vase. Biol. 16:591-599, Tjoelker, et al. (1995) Nature 374(6522):549-53), and Caslake et al. (2000) Atherosclerosis 150(2): 413-9. In addition, the protein, assays and methods of use have been described in the patent literature WO 95/00649-A1: U.S. Pat. Nos. 5,981,252, 5,968,818, 6,177,257, 7,052,862, 7,045,329, 7,217,535, 7,416,853; WO 00/24910-A1: U.S. Pat. Nos. 5,532,152; 5,605,801; 5,641,669; 5,656,431; 5,698,403; 5,977,308; and 5,847,088; WO 04/089184; WO 05/001416: U.S. Pat. No. 7,531,316; WO 05/074604; WO 05/113797; the contents of which are hereby incorporated by reference in their entirety.

Lp-PLA₂ is a Ca²⁺ independent member of the phospholipase A₂ enzyme family (Stafforini et al. 1997). Phospholipases are a broad group of enzymes that hydrolyze phospholipids into fatty acids and other lipophilic molecules. They are ubiquitously expressed with individual phospholipase family members having diverse biological functions including roles in inflammation, cell growth, signaling and death and maintenance of membrane phospholipids.

Lp-PLA2 circulates bound mainly to LDL, co-purifies with LDL, and is responsible for substantially all (>95%) of the phospholipase activity associated with LDL. It was initially characterized by its ability to hydrolyze and inactivate platelet-activating factor (PAF), a biologically potent phospholipid involved in inflammation (Tew et al. 1996). The Lp-PLA2 enzyme is produced by inflammatory macrophages, and shows increased expression in atherosclerotic lesions (MacPhee et al. 1999). The enzyme hydrolyzes the sn-2 fatty acid of oxidized LDL, resulting in the formation of lysophosphatidylcholine (lyso-PC) and oxidized free fatty acids (ox-FFA) (Carpenter et al. 2001; C. C. Hsieh et al. 2000; MacPhee et al. 1999; Shi et al. 2007; Takahashi et al. 2002). These reaction products are potent cytokines which in turn amplify and perpetuate the inflammatory cascade (Glass and Witztum 2001).

Multiple prospective epidemiology studies have suggested positive associations between Lp-PLA₂ measurements (both activity and mass) and risk of future CV morbidity and mortality. Unfortunately, consistent interpretation of these studies has proven difficult, particularly after adjusting for baseline concentrations of lipids and apolipoproteins. This variability of interpretation has prevented the adoption of general population risk assessment based on Lp-PLA₂ concentration or activity. See Rosen and Stafforini, “Role of Lipoprotein-Associated Phospholipase A2 in Vascular Disease (Chapter 13 of Clinical Lipidology: A Companion to Braunwald's Heart Disease (Elsevier Health Sciences, Nov. 14, 2014), p. 159). In general, more is known about Lp-PLA₂ mass (concentration) than activity. Although a cut point has been suggested for the mass assay (see, e.g., Lanman et al., “Lipoportein-Associated Phospholipase A2: Review and Recommendation of a Clinical Cut Point for Adults” in Preventive Cardiology, pp. 138-143 (Summary 2006)), this cut point (235 ng/ml) is specific to the mass (concentration assay) and has not been widely adopted, particularly across different assays, where variability has proven problematic. Further, this cut point is not applicable to the activity assay, particularly in light of the poor correlation between the activity and mass assays.

Recently, however, this association across many such studies has been the subject of an extensive analysis by the Lp-PLA₂ Studies Collaboration (LSC) in 2010 (Thompson et al. 2010). The LSC was a meta-analysis of 32 prospective studies, including individual records from 79,036 participants experiencing 17,722 incident fatal or non-fatal clinical outcomes during an aggregate 474,976 person-years. It consisted of both people with no history of vascular disease at baseline and patients with a history of stable vascular disease, the former of which is similar to the population studied in the REGARDS Clinical Validation Study, described below. Lp-PLA₂ measurements showed continuous association with risk for CHD events.

Cardiovascular disease remains the primary cause of premature mortality and morbidity in the United States, with one in six deaths due to coronary heart disease. Approximately 900 thousand heart attacks (myocardial infarctions) and 400 thousand coronary heart disease deaths occur annually in the US, with total direct and indirect costs estimated at over 315 billion dollars in 2010 (Go, Mozaffarian, Roger, Benjamin, Berry, Blaha, Dai, Ford, Fox, Franco, Fullerton, Gillespie, Hailpern, J. A. Heit, et al. 2014).

Atherosclerosis is the primary contributor to coronary heart disease. Atherosclerosis progresses as a consequence of unremitting inflammation in the vascular wall with an expanding lipid-laden necrotic plaque core and a thinning fibroatheromatous cap on the most vulnerable plaque (Hakkinen et al. 1999; Kolodgie et al. 2006). With plaque rupture and exposure of inflammatory products to the blood, the clotting cascade is immediately activated with risk of total vascular occlusion at the site of the plaque rupture with downstream ischemia and tissue necrosis. This is the etiology of most myocardial infarctions and about half of strokes (Ross 1999; Davies 2000).

Oxidation of low-density lipoprotein (LDL) in the arterial endothelium is understood to be the initiating event in the genesis of atherosclerotic plaque (Chisolm and Steinberg 2000; Witztum 1994). Oxidation of LDL initiates an inflammatory cascade within the arterial wall leading to the formation of the atherosclerotic lesion. Early atherosclerosis is characterized by an infiltration of monocytes into the arterial wall and their subsequent transformation into inflammatory macrophages which phagocytize and break-down the oxidized LDL. Macrophages become engorged with cholesterol, microscopically identifiable as cholesterol crystals, in the transformation into larger foam cells, which with apoptosis form the necrotic core of the vulnerable atherosclerotic plaque.

Multiple biomarkers, both demographic clinical parameters and blood serum measurements, are typically used in a physician's assessment of cardiovascular disease and the risk of coronary heart disease events. Such biomarkers are used both alone (as individual biomarkers) and together (with other biomarkers) for a more holistic assessment of patient risk.

The current recommendations for assessing heart disease risk in asymptomatic adults without prior history of disease use risk calculations that give predictions of the 10 year absolute risk (probability) of future CHD events. These predictions are generally made using an individual's demographic and serum biomarker data, such as age, gender, smoking history, diabetes, hypertension, and the serum measurement of total cholesterol, or its components LDL and HDL, as inputs. Examples of these predictive calculations include the Framingham Risk Score, FRS (Wilson et al. 1998), the Adult Treatment Panel III risk formulas (National Cholesterol Education Program 2002), and, most recently, the Pooled Cohort ASCVD Risk Equations (Goff et al. 2013). These risk calculators also vary in the endpoints used for risk prediction; from “hard” CHD events only to broader cardiovascular composite endpoints (the last calculator mentioned also includes non-coronary events such as stroke in a risk prediction of overall cardiovascular disease).

Such risk calculations used are derived from one or more large observational cohort studies, and are typically translated into a risk percentage or score based on the observed percentage of like participants experiencing a CHD event during the study (Eckel et al. 2013). Patients are assigned a calculated risk percentage and then grouped into three or more risk categories (for example, low, intermediate and high risk) which then inform provider recommendations for therapy, including lifestyle modification or pharmacotherapy, commensurate with their predicted level of CHD risk (Stone et al. 2013). Each such recommendation is further conditioned upon the specific modifiable risk factors and biomarker results of the individual patient; for example, anti-hypertension medications generally are prescribed based on an individual's blood pressure measurements, lipid-lowering medications such as statins in light of an individual's cholesterol measurements, and so on.

Together with these inputs and their own medical judgment, the provider develops a management plan for recommendation and discussion with the patient. This plan may be periodically revisited and revised as patient status changes over time; typically, in the absence of specific risk conditions, routine risk re-assessment is recommended every two to five years for this population as part of primary prevention standard guidelines (Goff et al. 2013).

However, the majority of individuals who experience an acute myocardial infarction (MI) have unremarkable serum cholesterol levels and are in fact classified by such risk scores as having low or intermediate risk (Sachdeva et al. 2009). More than half of sudden deaths from myocardial infarction occurs in patients with no prior history of CHD (Go et al. 2014), and most CHD events occur in a patients that would have been categorized as low or intermediate risk by standard risk calculator prior to their event (Naghavi et al. 2006). These observations underscore the need for better risk stratification tools given that there are effective therapies for reducing CHD events, particularly when applied before the development of symptomatic disease, and given the often catastrophic and deadly nature of such events.

Past studies have shown higher levels of Lp-PLA2 in men than in women without assessing the prognostic implications of the LpPLA2 measurements on CVD risk prediction, or the clinical implications of the observed sex difference in Lp-PLA2 (Brilakis et al. 2008). A recent study found that when fully adjusted for other risk factors Lp-PLA2 mass, but not activity, was independently associated with CVD in women (Cook et al. 2012). These observations underscore the need for better understanding of the difference in genders and if Lp-PLA2 activity levels should be interpreted differently in men or women, or if different cut points should be used for men and women when evaluating Lp-PLA2 activity levels for CVD risk assessment.

Some publications have indicated that Lp-PLA2 activity levels should be interpreted differently based on the patient's race. (Lee et al. 2011). One study found that compared with black subjects, adjusted Lp-PLA2 activity was higher in white and Hispanic subjects, respectively. (Brilakis et al. 2008). These observations also underscore the need for better understanding of the difference in races and if Lp-PLA2 activity levels should be interpreted differently in different races, or if different cut points should be used for different races when evaluating Lp-PLA2 activity levels for CVD risk assessment.

A low-frequency homozygous null mutation (V279F) has been reported in individuals with two Asian parents, resulting in a low level of circulating Lp-PLA2.

There are a handful of tests, both mass (e.g., ELIZA-type) assays and activity (e.g., enzymatic activity) assays that have been described. For example, the United States Food and Drug Administration (FDA) has granted clearance for the PLAC® Test (diaDexus, South San Francisco, Calif.), a mass assay, for the quantitative determination of Lp-PLA2 in human plasma or serum, to be used in conjunction with clinical evaluation and patient risk assessment as an aid in predicting risk for coronary heart disease, and ischemic stroke associated with atherosclerosis. Additional Lp-PLA2 assays include: the PLAC® Test for Lp-PLA2 Activity (diaDexus, South San Francisco, Calif.) available in Europe as a self-certified CE marked product for use in conjunction with clinical evaluation and patient risk assessment as an indicator of atherosclerotic cardiovascular disease, (Caslake, 2000), PAF activity assays (PAF Acetylhydrolase Assay Kit, Cat#760901 product brochure, Cayman Chemical, Ann Arbor, Mich., Dec. 18, 1997); Azwell/Alfresa Auto PAF-AH kit (available from the Nesco Company, Alfresa, 2-24-3 Sho, Ibaraki, Osaka, Japan or Karlan Chemicals, Cottonwood, Ariz., see also Kosaka (2000)), spectrophotometric assays for serum platelet activating factor acetylhydrolase activity (Clin Chem Acta 296: 151-161, WO 00/32808 (to Azwell)), and other published methods to detect Lp-PLA2 include WO 00/032808, WO 03/048172, WO 2005/001416, WO 05/074604, WO 05/113797, U.S. Pat. Nos. 5,981,252 and 5,880,273 and U.S. publication No. US 2012-0276569 A1.

Lp-PLA2 activity shows continuous association with risk of coronary heart disease events (Thompson et al. 2010). Since increasingly higher Lp-PLA2 activity levels are associated with increasingly higher risk of a CHD event, as a biomarker for risk assessment Lp-PLA2 activity has been evaluated on a continuous scale, not against a binary cut point. Commonly, the continuous scale is broken into arbitrary ranges of Lp-PLA2 activity, such as tertiles and quartiles, to aid clinicians with evaluating CHD risk (e.g., ≦151 nmol/min/mL=low risk, 152-194 nmol/min/mL=medium risk, ≧195 nmol/min/mL=high risk). However, these groups of low, moderate and high risk on the continuous scale may not clearly inform a clinician or patient if new, continued or increased therapeutic intervention should be taken; for example, is a reduction of high to moderate risk sufficient? Today, many people with CHD or at risk for CHD do not get identified or appropriately treated. As such, existing diagnostic methods using Lp-PLA2 may be problematic. Thus, there is a need for more reliable assays and treatment methods, as well as more effective methods to identify and stratify individuals at risk of CHD.

Described herein are methods and assays for determining a simple binary cut point grounded by clinical evidence that Lp-PLA2 activity levels at or above 225 nmol/min/mL (measured with a PLAC activity assay or other activity assay normalized to this cut point) is robustly associated with an increased risk of CHD, while activity levels below the cut point are associated with a lower risk of CHD. The cut point described herein, and the associated methods of identifying and guiding treatment of a patient based on this cut point, may be reliably and simply used for both men and women, and across races, despite previous reports. Further, the methods described herein may be used.

### SUMMARY OF THE DISCLOSURE

Described herein are methods and assays (including systems, kits, software/firmware, etc.) for determining or identifying, treating, and diagnosing coronary heart disease events and/or risk based on the activity of Lp-PLA₂ from patient samples (e.g., blood samples). The methods and assays described herein, unlike previously described Lp-PLA2 based assays, provide a robust binary cut point specific to the detected Lp-PLA₂ activity. In particular, the methods described herein may be relevant to patients of any race and/or gender to guide treatment of CHD. The detailed description below provides examples and illustrations, and also describes a group of clinical studies that support claimed methods and assays based on the cut point described herein. These clinical studies demonstrate the clinical effectiveness and performance characteristics of the methods and apparatuses described herein and validate that the Lp-PLA₂ activity assays and methods described are informative predictors (prognostic markers and risk factors) of coronary heart disease events (CHD) in patients with no prior history of cardiovascular events, including both binary classifiers (using a pre-specified analysis cut point) and also as continuous measures of risk (over ranges of values).

For example, described herein are methods of treating a patient for coronary heart disease (CHD). These methods may include performing or requesting a test providing the amount of lipoprotein-associated phospholipase A2 (Lp-PLA2) activity in a patient sample (the test may include: contacting a portion of the patient sample with an artificial substrate for Lp-PLA2 to enzymatically degrade the substrate, detecting a signal that is proportional to the degradation of the artificial substrate for Lp-PLA2, and determining an amount of Lp-PLA2 activity based on the signal); determining that the amount of Lp-PLA2 activity is at or above a cut-point when the amount of Lp-PLA2 activity in the patient sample is greater than or equal to a cut-point comprising an amount equivalent to a PLAC Activity value of 225 nmol/min/mL; and treating the patient for CHD when the amount of Lp-PLA2 activity in the patient sample is at or above the cut-point.

As will be described in greater detail below, a cut-point comprising an amount equivalent to a PLAC Activity value of 225 nmol/min/mL may be determined by normalizing the PLAC Activity assay (described in detail below) to other assays. For example, relative cut points for known Lp-PLA₂ Activity assays different from the PLAC assay may be calculated based on comparison plots of test results from a broad range panel. Such assays may be performed on calibrator solutions of known activity and regression (e.g., linear regression) may be applied to predict the corresponding cut point equivalent to the 225 nmol/min/ml of a PLAC assay. Although variations in substrates, calibration and reaction temperatures may have impact on the final activity results for each test, normalization across assay platforms may be easily determined by comparison with the PLAC Assay to determine an equivalent cut point. For example, commercially available Azwell PAF-AH activity assays referenced above may have a cut point equivalent to a PLAC Activity value of 225 nmol/min/mL of approximately 551.9 IU/L. Commercially available Cayman PAF-AH activity assays may have a cut point equivalent to a PLAC Activity value of 225 nmol/min/mL of approximately 16.4 nmol/min/mL. Similarly, commercially available DiaSys Lp-PLA2 FS activity assays may have a cut point equivalent to a PLAC Activity value of 225 nmol/min/mL of approximately 532.0 U/L.

A method of treating a patient for coronary heart disease (CHD) may include: requesting or performing a test providing the amount of Lp-PLA2 activity in a patient sample, wherein the test comprises: contacting a portion of the patient sample with an artificial substrate for lipoprotein-associated phospholipase A2 (Lp-PLA2) to enzymatically degrade the substrate, detecting a signal that is proportional to the degradation of the artificial substrate for Lp-PLA2; determining that the amount of Lp-PLA2 activity is at or above a cut-point when the amount of Lp-PLA2 activity in the patient sample is greater than or equal to a cut-point comprising an amount equivalent to a PLAC Activity value of 225 nmol/min/mL; and administering an agent to treat coronary heart disease when the amount of Lp-PLA2 activity in the patient sample is at or above the cut-point.

Treating may generally comprise administering an agent to treat CHD. For example, the agent may be a device and/or a drug. In some variations, treatment may include administering one or more medications selected from the group consisting of: an Lp-PLA2 inhibitor, an anti-inflammatory agent, an anti-thrombotic agent, an anti-platelet agent, a fibrinolytic agent, a lipid reducing agent, a direct thrombin inhibitor, a glycoprotein II b/IIIa receptor inhibitor, an agent that binds to cellular adhesion molecules and inhibits the ability of white blood cells to attach, an aldosterone antagonist, an angiotensin-converting enzyme (ACE) inhibitor, am angiotensin-receptor blocker (ARB), aspirin, a beta blocker, digoxin, a diuretic, an inotrope, digitalis, hydralazine, nitrates, statins, and warfarin.

Treating the patient for CHD may comprise treating the patient of any race (or gender) when the amount of Lp-PLA2 activity in the patient sample is greater than or equal to 225 nmol/min/mL. As described in greater detail below, the cut point for activity of an amount equivalent to a PLAC Activity value of 225 nmol/min/mL has been determined by both modeling and by clinical data (shown below) to be surprisingly effective at guiding diagnosis and/or treatment across multiple variables including, most surprisingly, race. Previous work had suggested that racial (e.g., between Caucasians, Asians, and African-Americans, for example) differences in activity level and risk assessment would require different cut points. The cut-point comprising an amount equivalent to a PLAC Activity value of 225 nmol/min/mL is effective for both male and female subjects and is not race-dependent. Cut points below or above this value may be ineffective, and may result in a unacceptable amount of false positives or false negatives.

Thus, for example, also described herein are methods of treating a patient of any race for coronary heart disease (CHD), the method comprising: performing or requesting a test providing the amount of Lp-PLA2 activity in a patient sample, wherein the test comprises: contacting a portion of the patient sample with an artificial substrate for lipoprotein-associated phospholipase A2 (Lp-PLA2) to enzymatically degrade the substrate, detecting a signal that is proportional to the degradation of the artificial substrate for Lp-PLA2, and determining an amount of Lp-PLA2 activity based on the signal; and administering an agent to treat coronary heart disease to a patient of any race when the amount of Lp-PLA2 activity in the patient sample is greater than or equal to a cut-point comprising an amount equivalent to a PLAC Activity value of 225 nmol/min/mL.

Also described herein are apparatuses, including software, hardware, and/or firmware. For example, described herein are non-transitory computer-readable storage medium storing a set of instructions capable of being executed by a processor (including one or more, e.g., linked processors), that, when executed by the processor(s), causes the processor(s) to: detect a signal indicating a degree of degradation of an artificial substrate for lipoprotein-associated phospholipase A2 (Lp-PLA2) after the artificial substrate for Lp-PLA2 has been placed in contact with a patient sample; determine an activity level of Lp-PLA2 in the sample based on the signal; and indicate if the activity level is equal to or greater than about a cut point comprising an amount equivalent to a PLAC Activity value of 225 nmol/min/mL. The set of instructions, when executed by the processor(s), may cause the processor to output a report including the activity level. The output may be a report including the indicator that the activity level is equal to or greater than about the cut point.

The one or more processors may be connected to and/or part of an automated clinical chemistry analyzer.

In general, the set of instructions, when executed by the processor may cause the processor to detect a plurality of signals indicating degradation of the artificial substrate for lipoprotein-associated phospholipase A2 (Lp-PLA2) after the artificial substrate for Lp-PLA2 has been placed in contact with the patient sample. For example, the set of instructions, when executed by the processor may cause the processor to generate a standard curve using a curve fit model.

Also described herein are non-transitory computer-readable storage medium storing a set of instructions capable of being executed by a processor (or connected set of processors) may, when executed by the processor, causes the processor to: receive a signal indicating a degree of degradation of an artificial substrate for lipoprotein-associated phospholipase A2 (Lp-PLA2) after the artificial substrate for Lp-PLA2 has been placed in contact with a patient sample; determine an activity level of Lp-PLA2 in the sample based on the signal; and indicate if the activity level is equal to or greater than about a cut point comprising an amount equivalent to a PLAC Activity value of 225 nmol/min/mL.

In some variations the non-transitory computer-readable storage medium including instructions that receive data (e.g. signals) from additional devices (e.g., automatically or manually) and process the data to determine the enzymatic activity of the LpPLA2 and analyze it to guide treatment by a physician, for example, by providing a report on the patient's Lp-PLA2 activity. The output (e.g., report) may include one or more indicator that the activity is at or above the cut point, such as a PLAC Activity value of 225 nmol/min/mL. The indicator may be a visual (e.g. color), symbol, text, or some other indicator.

For example, described herein are non-transitory computer-readable storage medium storing a set of instructions capable of being executed by a processor, that, when executed by the processor, causes the processor to: receive an activity level of Lp-PLA2 from a sample, wherein the activity level was determined from a signal indicating a degree of degradation of an artificial substrate for lipoprotein-associated phospholipase A2 (Lp-PLA2) after the artificial substrate for Lp-PLA2 has been placed in contact with a patient sample; and output a report indicting if the activity level is equal to or greater than about a cut point comprising an amount equivalent to a PLAC Activity value of 225 nmol/min/mL.

Also described herein are methods of treating a patient for coronary heart disease (CHD), the method comprising: contacting a portion of a sample from the patient with an artificial substrate for lipoprotein-associated phospholipase A2 (Lp-PLA2) to enzymatically degrade the substrate; detecting a signal that is proportional to the degradation of the artificial substrate for Lp-PLA2; calculating an amount of Lp-PLA2 activity based on the signal; and treating the patient for CHD when the amount of Lp-PLA2 activity is greater than or equal to a cut point comprising an amount equivalent to a PLAC Activity value of 225 nmol/min/mL.

As mentioned, treating may comprises instructing a physician that the patient is at risk for coronary heart disease so that the physician may administer to the patient a therapy for CHD. For example, treating may comprise instructing a physician that the patient's Lp-PLA2 activity is greater than or equal to a cut point comprising an amount equivalent to a PLAC Activity value of 225 nmol/min/mL and the patient is at risk for coronary heart disease so that the physician may administer to the patient a therapy for CHD. Treating may comprise administering an agent to treat CHD. Treating may comprise administering one or more medications selected from the group consisting of: an Lp-PLA2 inhibitor, an anti-inflammatory agent, an anti-thrombotic agent, an anti-platelet agent, a fibrinolytic agent, a lipid reducing agent, a direct thrombin inhibitor, a glycoprotein II b/IIIa receptor inhibitor, an agent that binds to cellular adhesion molecules and inhibits the ability of white blood cells to attach, an aldosterone antagonist, an angiotensin-converting enzyme (ACE) inhibitor, am angiotensin-receptor blocker (ARB), aspirin, a beta blocker, digoxin, a diuretic, an inotrope, digitalis, hydralazine, nitrates, statins, and warfarin.

For example, a method of treating a patient of any race for coronary heart disease (CHD) may include: contacting a portion of a sample from the patient with an artificial substrate for lipoprotein-associated phospholipase A2 (Lp-PLA2) to enzymatically degrade the substrate; detecting a signal that is proportional to the degradation of the artificial substrate for Lp-PLA2; calculating an amount of Lp-PLA2 activity based on the signal; and treating the patient of any race for CHD when the amount of Lp-PLA2 activity is greater than or equal to a cut point comprising an amount equivalent to a PLAC Activity value of 225 nmol/min/mL.

Also described herein are methods for predicting the risk of a coronary heart disease event (CHD) for a patient comprising: contacting a portion of a sample from the patient with an artificial substrate for lipoprotein-associated phospholipase A2 (Lp-PLA2), thereby enzymatically degrading the substrate when contacted to Lp-PLA2, the substrate producing a detectable signal when contacted to Lp-PLA2; quantifying the detectable signal produced in the contacting step, the signal being proportional to the Lp-PLA2 enzymatic activity in the sample, whereby an Lp-PLA2 activity is calculated; comparing the Lp-PLA2 activity determined in the quantifying step to a reference amount; and providing a risk of CHD for the patient if the amount Lp-PLA2 activity in the sample is equal to or greater than the Lp-PLA2 activity reference. The reference amount may be a binary cutoff; for example, the reference amount may be approximately (or exactly) 225 nmol/min/mL (e.g., equivalent to a PLAC Activity value of 225 nmol/min/mL). As mentioned, the method may also include treating the patient for CHD if the amount Lp-PLA2 activity in the sample is equal to or greater than a PLAC Activity value of 225 nmol/min/mL, for example, by administering an agent to treat CHD treating the patient for CHD if the amount Lp-PLA2 activity in the sample is equal to or greater than a PLAC Activity value of 225 nmol/min/mL. The agent may be one or more medications selected from the group consisting of: an Lp-PLA2 inhibitor, an anti-inflammatory agent, an anti-thrombotic agent, an anti-platelet agent, a fibrinolytic agent, a lipid reducing agent, a direct thrombin inhibitor, a glycoprotein II b/IIIa receptor inhibitor, an agent that binds to cellular adhesion molecules and inhibits the ability of white blood cells to attach, an aldosterone antagonist, an angiotensin-converting enzyme (ACE) inhibitor, am angiotensin-receptor blocker (ARB), aspirin, a beta blocker, digoxin, a diuretic, an inotrope, digitalis, hydralazine, nitrates, statins, and warfarin.

Also described are method for diagnosing (and/or treating) coronary heart disease event (CHD) for a patient that include examining the man-made intermediate (e.g. a complex of the Lp-PLA2 from the patient's sample and an artificial substrate for the Lp-PLA2. For example, a method may include obtaining a blood sample from the patient; combining the blood sample with an artificial substrate for lipoprotein-associated phospholipase A2 (Lp-PLA2), wherein the presence of the artificial substrate for Lp-PLA2 creates a transient complex of artificial substrate and Lp-PLA2 that degrades the artificial substrate; detecting the degradation of the artificial substrate resulting from the transient complex of artificial substrate and Lp-PLA2; quantifying an activity of the Lp-PLA2 in the sample from the detected degradation of the artificial substrate; and indicating a risk of CHD for the patient if the activity of the Lp-PLA2 in the sample in is equal to or greater than an Lp-PLA2 activity reference. As mentioned above, the Lp-PLA2 activity reference may be a binary cutoff, such as a PLAC Activity value of 225 nmol/min/mL. The method may also include treating the patient for CHD when activity of the Lp-PLA2 in the sample in is equal to or greater than a PLAC Activity value of 225 nmol/min/mL, e.g., by administering an agent to treat CHD.

Also described are methods for diagnosing coronary heart disease event (CHD) for a patient, the method comprising: obtaining a blood sample from the patient; combining the blood sample with an artificial substrate for lipoprotein-associated phospholipase A2 (Lp-PLA2), wherein the presence of the artificial substrate for Lp-PLA2 creates a transient complex of artificial substrate and Lp-PLA2 that degrades the artificial substrate; detecting the degradation of the artificial substrate resulting from the transient complex of artificial substrate and Lp-PLA2; quantifying an activity of the Lp-PLA2 in the sample from the detected degradation of the artificial substrate; and indicating a risk of CHD for the patient if the activity of the Lp-PLA2 in the sample in is equal to or greater than a PLAC Activity value of 225 nmol/min/mL.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings. The figures include:

FIGS. 1A and 1B are Kaplan Meier survival plots describing a first and second (respectively) training cohort used with the methods and assays described herein (showing incidence of chronic heart disease, CHD, in subjects with no prior cardiovascular disease from the REGARDS data). In both FIGS. 1A and 1B, the log-rank test between groups: p<0.0001, top line represents subgroup with Lp-PLA2 Activity <225 nmol/min/mL (using the PLAC Activity assay for Lp-PLA2 activity); bottom line represents subgroup with Lp-PLA2 Activity ≧225 nmol/min/mL (using the PLAC Activity assay).

FIG. 2 diagrammatically illustrates experimental parameters for some of the examples described herein, including the construction of clinical validation study case-cohort validating the cut point from the REGARDS data.

FIG. 3 and FIG. 4 illustrate parameters of the case-cohort as described herein, which was used to verify the cut point of the PLAC Activity value of 225 nmol/min/mL from the REGARDS data.

FIGS. 5A-5C illustrate PLCA assay distribution by gender from the REGARDS data.

FIG. 6 illustrates PLAC assay results by race and by gender from the REGARDS data.

FIG. 7 shows REGARDS Population Reference Range by Age (Total).

FIGS. 8A-8B illustrate REGARDS Population Reference Range by Age, showing Males (FIG. 8A) and Females (FIG. 8B) from the REGARDS data.

FIG. 9 shows a Kaplan-Meier Analysis of the absolute risks and log-Rank Test for the entire sample, for subjects above and below the cut point from the REGARDS data.

FIGS. 10A-10B show Kaplan-Meier Analysis of the absolute risks and log-rank tests for Caucasians and African-American's, respectively for subjects above and below the cut point from the REGARDS data.

FIGS. 11 and 12 show Kaplan-Meier Analyses—Pre-Test Absolute Risk of CHD Events from the REGARDS data.

FIG. 13A shows cox analyses indicating univariate hazard ratios by race from the REGARDS data.

FIG. 13B shows cox analyses indicating univariate hazard ratios from the REGARDS data.

FIG. 14 shows an interaction test illustrating the PLAC Activity Group by race from the REGARDS data.

FIG. 15A illustrates a cox analyses showing multivariate hazard ratios from the REGARDS data.

FIG. 15B shows an extensively risk-adjusted cox model from the REGARDS data.

FIGS. 16A and 16B illustrate subgroup analyses by gender (Kaplan Meier Analyses), showing males (FIG. 16A) and Females (FIG. 16B) from the REGARDS data using the cut point described herein.

FIG. 17 illustrates a subgroup analysis by gender showing Cox hazard ratios from the REGARDS data using the cut point described herein.

FIG. 18 illustrates a subgroup analysis by race showing Cox hazard ratios from the REGARDS data using the cut point described herein.

FIG. 19 illustrates secondary component endpoints (univariate hazard ratios) from the REGARDS data using the cut point described herein.

FIG. 20 shows a Kaplan-Meier analysis of myocardial infarction (MI) from the REGARDS data using the cut point described herein.

FIG. 21 shows a Kaplan-Meier analysis of coronary revascularization from the REGARDS data using the cut point described herein.

FIG. 22 shows a Kaplan-Meier analysis of CHD-related death from the REGARDS data using the cut point described herein.

FIG. 23 shows a Kaplan-Meier analysis of PLAC activity tertiles (absolute event rates and log-rank test) from the REGARDS data.

FIGS. 24A and 24B show Kaplan-Meier analyses of PLAC Activity tertiles by race, showing Caucasians (FIG. 24A) and African-Americans (FIG. 24B) from the REGARDS data.

FIG. 25 is a cox analysis showing the hazard ratios (continuous PLAC activity) from the REGARDS data.

FIG. 26 shows an extensively risk-adjusted Cox Model (Continuous PLAC Activity) from the REGARDS data.

FIGS. 27A, 27B and 27C show PLAC activity values by outcome subgroup (CRS, controls, and total cases respectively) from the REGARDS data.

FIG. 28 shows PLAC Activity values by race and outcome subgroups from the REGARDS data.

FIGS. 29A, 28B and 29C show PLAC Activity by Gender and Outcome for the full analysis set (FAS), FAS Males, and FAS Females, respectively from the REGARDS data.

FIGS. 30A, 30B and 30C show PLAC Activity by Gender and Outcome for African Americans, African American Males, and African American Females, respectively from the REGARDS data.

FIGS. 31A, 31B and 31C show PLAC Activity by Gender and Outcome for Caucasians, Caucasian Males, and Caucasian Females, respectively from the REGARDS data.

FIG. 32 shows gender and race subgroups from a normal reference range study.

FIGS. 33A and 33B show the male and female subgroups (respectively) from the normal reference range study.

FIGS. 34A, 34B and 34C show normal reference range study by age data for total, males and females, respectively.

FIG. 35A shows a graph of PLAC Activity distribution by race.

FIG. 35B shows a table of total PLAC Activity by race.

FIG. 36A shows a graph of PLAC Activity distribution by gender for males.

FIG. 36B shows a table of total PLAC Activity by gender for males.

FIG. 37A shows a graph of PLAC Activity distribution by gender for females.

FIG. 37B shows a table of total PLAC Activity by gender for females.

FIGS. 38A and 38B show PLAC Activity by race for males and females, respectively.

FIG. 39 shows a case-cohort clinical characteristic by race.

FIG. 40 is a table illustrating dataset variable specifications, as described herein.

FIG. 41 is a table comparing cut points across different Lp-PLA2 activity assays, including the PLAC Activity value of 225 nmol/min/mL.

### DETAILED DESCRIPTION

In general, described herein are methods and assays (including compositions, kits, systems, etc.) for detecting, identifying, prognosticating, preventing, stratifying, and treating coronary heart disease (CHD). In particular, described herein are methods and assays for use assessing, detecting, identifying, prognosticating, preventing, stratifying, and treating risk of CHD events may including myocardial infarction, coronary revascularization, and CHD-related death. The method and assays for analyzing risk of CHD typically use a simple cut point to evaluate Lp-PLA2 activity of a biological sample (e.g., blood, plasma, serum, whole blood, etc.). A method or assay may analyze the activity of Lp-PLA2 to determine if an individual has or is at risk of having CHD. Further, the assay or method may analyze the activity of the Lp-PLA2 against a specified cut point to identify the individual as either high or low risk for CHD. Such a test is an improvement over existing methods where no cut point is established and CHD risk assessment is based on a sliding scale to determine if Lp-PLA2 activity level is high. A simple assay with a single cut point dividing individuals into high CHD risk and low CHD risk may reduce the need for other expensive methods to assess risk. Further, the test with single cut point will enable selection and administration of costly therapies to reduce risk of CHD to those individuals who will benefit from such therapies.

The following terms and abbreviations may be used to described the clinical performance analyses and/or the PLAC Activity assay (PLAC Test) for LP-PLA₂ Activity: ACS (“Acute Coronary Syndromes”); AHA (American Heart Association); AMI or MI (Acute Myocardial Infarction); BAS (Black Analysis Set of the Clinical Validation Study, consisting of the African-American subjects only); FAS (Full Analysis Set of the Clinical Validation Study); HDL (High-density lipoprotein); HR (Hazard ratio); IRB (Institutional Review Board); LDL (Low-density lipoprotein); NHIS (National Health Interview Survey); NHLBI (National Heart Lung and Blood Institute); NIH (National Institutes of Health); NINDS (National Institute of Neurological Disorders and Stroke).

As used herein, baseline may refer to, at patient presentation or entry into a clinical study; typically, the earliest available patient characteristics in a study. For the studies presented here, the baseline date is consistently analyzed as the blood draw date of the subject sample tested. The term “case” may refer to a subject that experienced a clinical endpoint during a study. The term case-cohort cases may refer to the full set of cases analyzed in the Case-Cohort Study. Represents 933 of the total 1,028 Primary Clinical Endpoint (CHD) cases in the full Indicated REGARDS Population. This set includes both 152 CHD cases randomly selected in the Cohort Random Sample, as well as 781 additional CHD cases enriched from the remainder of the Indicated REGARDS Population.

As used herein, the case-cohort study refers to the study design used in the Clinical Validation Study efficacy analyses, in which characteristics of cases are compared with controls from a population cohort sample. In brief, a population cohort sample is selected from a parent study (parent study or parent cohort), with the controls and cases from this sample then further enriched with additional cases from the parent study. Case-cohort study analyses typically utilize sample weights in order to arrive at a representation of the overall parent study prevalence rates. In general, a control may refer to a subject that did not experience a clinical endpoint, e.g., during a study.

The term CHD may refer to coronary heart disease events, specifically acute myocardial infarction (MI), coronary revascularization procedures and CHD related death events. The term CI typically refers to confidence interval; in the analysis below, CIs shown may represent the 95 percentile confidence intervals (see LCI and UCI below). Clinical Program may refer to the complete clinical program described herein, including both the longitudinal Clinical Validation Study and cross-sectional population Reference Studies.

A Clinical Validation Study may refer to the longitudinal study used for clinical efficacy analyses (the Case-Cohort Study) and other clinical performance analysis of the product (selected subpopulations, as indicated), which was selected from the Indicated REGARDS Population. A Cohort Random Sample (CRS) may refer to the subset of the Clinical Validation Study that was sampled from the Indicated REGARDS Population using an equal probability selection method (EPSEM) procedure (without stratification or other criteria). Represents 3,817 subjects selected from the 23,019 subjects in the Indicated REGARDS Population.

As used herein, a Cohort Study refers to an observational study in which groups of patients are identified and followed up over time (longitudinal). Patient selection may occur at the point of enrollment and is usually based upon demographic criteria or exposure to an environmental or medical condition. A Continuous Analysis typically refers to statistical analysis of the PLAC Activity value across its reported range as a continuous predictor of the clinical endpoint of interest.

As used herein, a Cox Model refers to the Cox proportional hazards model.

Cut point Analysis may refer to a statistical analysis of subject PLAC Activity group membership as a dichotomous predictor of the specified Primary or Secondary Clinical Endpoint; in other words, using Lp-PLA2 Activity as a categorical classifier, grouping subjects into one of two binary categories based on their Lp-PLA2 Activity being either (i) less than the Analysis Cut point (the “low” Lp-PLA2 Activity group) or (ii) equal to or greater than the Analysis Cut point (the “high” Lp-PLA2 Activity group”).

CVD refers to cardiovascular disease events, which include acute stroke and CHD events.

As used herein, endpoint may refer to the clinical event or value used as the overall measurement of a clinical trial, usually acute events, deaths, severe toxicity, or other measure of the presence or the progression of disease.

The phrase high PLAC Activity Group may refer to subjects with a PLAC Activity value of nmol/min/mL (the Analysis Cut point).

Indicated REGARDS Population typically refers to the subpopulation of the REGARDS study participants having no prior history of cardiovascular events (CHD or stroke) at baseline. Represents 23,019 of the 30,183 total REGARDS participants.

The term IPW refers to the inverse probability weight, a sample weighting methodology that may be used in case-cohort and other clinical study designs, using the inverse of a subject's probability of being sampled from a parent population as that subjects weight.

LCI (lower confidence interval) may refer to the lower defined boundary of a probability distribution for a statistical result (herein consistently using a 95% CI). UCI (upper confidence interval) typically refers to the higher defined boundary of a probability distribution for a statistical result (herein a 95% CI).

The term Log 2(Lp-PLA2) typically refers to the Log 2 (base 2) transformed value of a PLAC Lp-PLA2 Assay measurement, used in Continuous Analyses.

A Low PLAC Activity Group may refer to subjects with a PLAC Activity value of <225 nmol/min/mL (below the Analysis Cut point).

As mentioned above, Lp-PLA2 (Lp-PLA₂) refers to Lipoprotein-associated phospholipase A2 (Lp-PLA2), also known as platelet activating factor acetylhydrolase (PAF-AH), The enzymatic activity level of Lp-PLA2 is the measurand for the subject test. A 45-kDa protein encoded in humans by the PLA2G7 gene. Depending on the context, the term PLAC Activity may refer to an Lp-PLA2 activity measurement (in nmol/min/mL) made using the PLAC® Test for Lp-PLA2 Activity.

The term PLAC Activity Test typically refers to the PLAC Test for Lp-PLA2 Activity, which is described in greater detail below.

As used herein, a primary clinical endpoint may refer to the primary clinical endpoint used in the Clinical Validation Study, a composite endpoint of coronary heart disease events, specifically acute myocardial infarction, coronary revascularization procedures and CHD-related death events.

A reference study may refer to a population-based study employing a single point of data collection for each subject being studied, used in the Clinical Program to establish population reference ranges for Lp-PLA2 activity values for the unselected general population and for selected races.

As used herein, REGARDS refers to the Reasons for Geographic And Racial Differences in Stroke study, the broad national U.S. population parent cohort study from which the Indicated REGARDS Population and the Clinical Validation Study case-cohort is selected. The study is a cross-sectional general population study(s) used to describe the expected reference range intervals for PLAC Activity.

A risk score typically refers to a global risk score calculation (such as the Framingham Risk Score), used to estimate an individual's global risk of experiencing coronary heart disease events within the next 10 years. Typical variables used to calculate risk scores include age, gender, low-density (LDL) and high-density (HDL) lipoproteins, smoking, diabetes, and hypertension.

A normal reference cohort may refer to the primary reference cohort used both for setting the analysis cut point as well as defining the normal expected PLAC Lp-PLA2 Assay reference ranges in an unselected population.

As used herein SAP (Statistical Analysis Plan) refers to the SAP used in the Clinical Validation Study.

A secondary clinical endpoint typically refers to other pre-specified clinical endpoints used as clinical outcome variables in the Clinical Validation Study, which were analyzed independently in supportive analyses. These comprised each of the component CHD event types contained within the Primary Clinical Endpoint composite, specifically (i) acute myocardial infarction, (ii) coronary revascularization procedures, and (iii) CHD-related death.

As used herein, WAS is the White Analysis Set of the Clinical Validation Study, consisting of the Caucasian subjects only.

The phrase without CVD at baseline refers to subjects enrolled in REGARDS-MI with no evidence of a prior history of CVD events at time of enrollment.

In general, the term analysis cut point (or simply “cut point”) may refer to a PLAC Activity value of nmol/min/mL.

The PLAC Test for LP-PLA2 Activity

The PLAC® Test for Lp-PLA2 Activity (referred to elsewhere in this section simply as “PLAC Activity”, as measured by the “PLAC Activity Test”) is summarized as follows:

Description.

The PLAC Test for Lp-PLA2 Activity is an enzyme assay which consists of reagents, calibrators and controls for the measurement of Lp-PLA2 activity in serum and plasma on automated clinical chemistry analyzers.

Intended Use.

The PLAC Test for Lp-PLA2 Activity is an enzyme assay for the in vitro quantitative determination of Lp-PLA2 (lipoprotein-associated phospholipase A2) activity in EDTA-plasma and serum on automated clinical chemistry analyzers. Lp-PLA2 activity is to be used in conjunction with clinical evaluation and patient risk assessment as an aid in predicting risk of coronary heart disease (CHD) in patients with no prior history of cardiovascular events.

Calibrators.

The Lp-PLA2 Activity Test Calibrators are intended to establish points of reference that are used in the determination of values in the measurement of Lp-PLA2 activity by the PLAC Test for Lp-PLA2 Activity.

Controls.

The Lp-PLA2 Activity Test Controls are intended for use as a quality control tool to monitor the performance within the clinical range of the PLAC Test for Lp-PLA2 Activity, an enzyme assay for the quantitative determination of Lp-PLA2 activity.

Clinical Program and Interpretation of Results.

The results of the Lp-PLA2 Clinical Program are clinical evidence validating the product's clinical use. As described herein, an assay for Lp-PLA2 activity with a cut point of 225 nmol/min/mL was found to demonstrate utility in assessing risk of CHD events in patients. Specifically, the PLAC Activity Test demonstrated utility to assess risk of CHD events when used in a method that compared measured Lp-PLA2 activity levels to a cut point of 225 nmol/min/mL.

Principle of the Test.

The PLAC Test for Lp-PLA2 Activity is an enzyme assay. Lp-PLA2, in serum or plasma, hydrolyzes the sn-2 position of the substrate, 1-myristoyl-2-(4-nitrophenylsuccinyl) phosphatidylcholine, producing a colored reaction product, 4-nitrophenol. The rate of formation of 4-nitrophenol is followed spectrophotometrically and the Lp-PLA2 activity is calculated from the rate of change in absorbance. A set of five Lp-PLA2 calibrators is used to generate a standard curve fit of change in absorbance versus Lp-PLA2 activity level in nmol/min/mL from which the sample Lp-PLA2 activity is derived.

The clinical implementation of this test following regulatory clearance follows the use of other traditional cardiovascular risk biomarkers. Specifically, health care providers performing risk assessment for a given patient order a Lp-PLA2 activity test to assess the patient's risk of CHD. Further, healthcare providers may place the Lp-PLA2 activity results in context with other clinical and demographic indicators (e.g. age, gender, smoking, etc.) and with traditional cardiovascular risk factors (e.g. low density lipoprotein, total cholesterol, etc.) to refine their risk assessment in routine primary prevention. Alternately, the laboratory, hospital, clinic, office, or other party that runs the Lp-PLA2 activity test may make an assessment for a patient's CHD risk by reporting that the patient's Lp-PLA2 activity level is above or below a specified cut point. The reporting party may report the patient's risk in numerous ways including: enumerating the patient's Lp-PLA2 activity level and indicating it is above or below the cut point, color coding the patient's Lp-PLA2 activity level (e.g., green is below the cut point, red is above the cut point), and graphically representing the patient's Lp-PLA2 activity level in comparison to the cut point. Further, the reporting party may place the Lp-PLA2 activity results in context with other clinical and demographic indicators (e.g. age, gender, smoking, etc.) and with traditional cardiovascular risk factors (e.g. low density lipoprotein, total cholesterol, etc.) to refine or provide an overall risk assessment for the patient.

Example Assay Procedure

Calibration.

The assay is calibrated using a calibration curve. Multiple points may be used to generate the curve (i.e., a 2-point curve, a 3-point curve, a 4-point curve, a 5-point curve, a 6-point curve, a 7-point curve, a 8-point curve, etc.). A standard curve is generated using the appropriate curve fit model indicated in an application sheet specific to the analyzer on which the assay is being run. Verify the calibration with at least two levels of controls according to the laboratory's requirements. Recalibrate and run controls for each kit from a new lot and thereafter, every 4 weeks for kits from the same lot. If controls fall outside of laboratory's acceptable range, recalibrate as necessary up to the expiration date of the opened reagents.

Quality Control.

Test at least two levels of an appropriate quality control material a minimum of once per day for each day of use. In addition, run controls after each new calibration run. It is recommended that low and high controls be included in each run. If control values are not within acceptance limits, repeat the assay. Additional quality control testing may be necessary according to local, state and/or federal regulations or accreditation requirements.

Example Assay Procedure.

The PLAC Test for Lp-PLA2 Activity should be run using the appropriate settings for the analyzer to be used. For detailed instructions and settings for each analyzer, please refer to the analyzer application sheet for the specific automated clinical chemistry analyzer used. A general explanation of the assay procedure for the Beckman Coulter (Olympus) AU400® Analyzer is described:

| | |
|---|---|
| Assay Code | Rate |
| Assay Time | 8.5 minutes |
| Read Cycle | 12 to 14 |
| Sample Volume | 25 μL |
| Reagent R1 vol. | 100 μL R1 reagent (R1 position) |
| Reagent R2 vol. | 25 μL R2 reagent (R2 position) |
| Wavelength | 1° 410 nm, 2° 520 nm |
| Calibration Method | Spline 5 point |
| Assay Range | 10 to 400 nmol/min/mL |

Procedural Notes.

It is recommended that each lab determine a suitable calibration frequency. At a minimum, a new calibration curve should be generated with a kit from a new lot, and thereafter, every 4 weeks for kits from the same lot. Run calibration when and if controls fall outside acceptable range. Upon storage, do not switch caps on reagent solutions as this may lead to contamination. All samples should be well mixed before testing, and especially after thawing stored samples. A vortex mixer may be used; however, any air bubbles or foaming of the samples should be avoided.

Performance Characteristics

Performance characteristics were established using the Beckman Coulter (Olympus) AU400® Analyzer. Refer to the specific clinical chemistry Analyzer Application Sheet for performance characteristics.

Sensitivity.

The clinical sensitivity (limit of quantitation) of the assay is ≦10 nmol/min/mL.

Assay Precision.

Intra-assay and inter-assay variability were determined by testing four human serum samples and two controls with Lp-PLA₂ activities ranging from 95 to 345 nmol/min/mL. Samples were assayed in duplicate, twice a day, over 20 days and with 3 reagent lots. Total precision CV's for each reagent lot and sample were <3%. The minimum and maximum Intra-assay % CV (n=40) for the 3 lots was 1.1% and 2.0%, respectively. The minimum and maximum Inter-assay % CV (n=40) for the 3 lots was 1.1% and 2.6%, respectively.

Linearity.

Several dilution series were prepared from serum samples with known high and low Lp-PLA2 activity levels and tested with 3 lots of reagents. In the dynamic range of 8 to 393 nmol/min/mL, linear regression of Lp-PLA2 activity levels resulted in slopes ranging from 0.99 to 1.02, intercepts ranging from −2.5 to 5.1 nmol/min/mL and R2 ranging from 0.998 to 1.000. Linearity was demonstrated from 30 to 400 nmol/min/mL with a deviation from linearity of ≦10%. The measuring range of the assay is determined to be 10 to 400 nmol/min/mL with the low end of the range based on the limit of quantitation.

Interfering Endogenous Substances.

Endogenous substances were titrated into samples with known levels of each endogenous substance and were tested. No appreciable interference was observed for the following substances: Albumin (60 g/L), Bilirubin (20 mg/dL), Cholesterol (500 mg/dL), Triglycerides (300 mg/dL), Hemoglobin (1 mg/mL).

Interfering Exogenous Substances.

Exogenous substances (common and prescription drugs) were evaluated for interference in the assay. Samples were spiked with two levels of the potential interferent and tested. No appreciable interference was observed for the following substances at the spiked levels tested (low, high): Albumin, g/L (35, 50); Triglycerides mg/dL (150, 500); Bilirubin, mg/dL (0.2, 5); Cholesterol, mg/dL (150, 250); Hemoglobin, mg/mL (0.62, 1.00); Acetaminophen, μmol/L (33, 199); Aspirin, μmol/L (720, 3600); Atorvastatin, μmol/L (2, 20); Diphenhydramine, μmol/L (2, 20); Fenofibrate, μmol/L (42, 125); Lisinopril, μmol/L (0.25, 0.74); Niacin, μmol/L (480, 4800); Tolbutamide, μmol/L (400, 2300); Warfarin, μmol/L (10, 33); Metformin, μmol/L (31, 310); Clopidogrel bisulfate, μmol/L (10, 100); Vitamin C, μmol/L (14, 342).

Recovery.

Various amounts of a high Lp-PLA2 activity level solution were added to an enzyme-free diluent to create seven activity levels. These spiked solutions were assayed with 3 lots of reagents and the Lp-PLA2 activity levels were then compared to expected values resulting in slopes ranging from 0.99 to 1.10, Intercepts ranging from −2.9 to 4.2 nmol/min/mL and R2 ranging from 0.997 to 1.000.

Clinical Program Overview

The overall objective of the Clinical Program was to validate that Lp-PLA2 activity values are informative as an aid, in conjunction with clinical assessment, in predicting risk of coronary heart disease events in patients with no prior history of cardiovascular events. The PLAC Activity test was used in the Clinical Program to demonstrate this objective.

Clinical Studies.

The Clinical Program was comprised of three clinical studies: the longitudinal case-cohort study in the intended use population with no prior history of CVD (the “Clinical Validation Study”), and two cross-sectional reference population studies (the “Reference Studies”), consisting of the “Normal Reference Study” in an unselected normal population and a parallel combined study analysis of PLAC Activity across selected race subgroups (the “Racial Diversity Study”).

The Clinical Program generally follows a “prospectively planned retrospective” study design, whereby retrospective specimens, collected at an earlier baseline encounter and properly preserved, are acquired from relevant studies and then tested by the PLAC Activity Test on a Beckman Coulter (Olympus) AU400® clinical chemistry analyzer while blinded to patient demographic or outcome clinical data. The resulting testing data is later joined with the clinical data for statistical analyses. In the longitudinal Clinical Validation Study, subsequent CHD clinical outcome data over the subsequent “prospective” follow up period are also added for efficacy analyses. In the cross-sectional Reference Studies, such clinical data is available solely at the single baseline encounter, as the patient is not followed afterwards.

An aggregate of 5,706 subjects were tested using the PLAC Test for Lp-PLA2 Activity, with 4,598 subjects, including 933 CHD cases, in the Clinical Validation Study, and an additional 1,108 subjects in the Normal Reference Study. More detailed descriptions of the study cohorts, including baseline clinical characteristics and inclusion and exclusion criteria, are provided in the following sections provided for each study.

A summary overview of the entire Clinical Program by study is presented in Table 1 below.

TABLE 1

| Study | Study Design | Subjects Tested | Source |
|---|---|---|---|
| Clinical | Longitudinal case- | 4,598 | 48 US states, |
| Validation | cohort study in the | (3,817 Cohort | 23,019 subject |
| Study | intended use | Random Sample; | parent study |
| | population | 933 Total | (within intended |
| | | Cases) | use population) |
| Normal | Cross-sectional | 1,108 | 3 US recruitment |
| Reference | study in unselected | | sites |
| Study | normal | | |
| | population age and | | |
| | gender subgroups | | |
| Racial | Cross-sectional | 4,925 | Parallel analysis |
| Diversity | study in specific | (CRS Sample only | of combined |
| Study | racial population | plus Normal | population studies |
| | subgroupss | Reference | by race subgroups |
| | | Study) | |
| Total | | 5,706 | |
| Subjects | | | |

Notes:
Ancillary and analytical studies covered in other sections of this filing are not shown above.

The specific design, including any inclusion and exclusion criteria, of the studies are discussed in further detail in each relevant study result section. Statistical methods used were pre-specified for the Clinical Validation Study prior to un-blinding data and beginning analysis; Reference Studies generally followed methods in FDA-recognized guidelines (FDA Recognition Number 7-224) from the Clinical and Laboratory Standards Institute (CLSI 2010), including the guideline application to specific partitioned subgroups of gender, age, and race.

Common Study Requirements.

All studies met certain common perquisites prior to their inclusion in the Clinical Program.

Institutional Review Board Approval and Patient Informed Consent.

Institutional Review Board (IRB) approvals for the source cohorts contained purposes consistent with the Clinical Program; all patients provided informed consent.

Consistent Sample Handling and Storage.

Sample processing consistent with the proposed instructions for use, with measures for continued sample integrity and storage at −70° C. (or below) by principal investigator or Sponsor through to the time of PLAC Activity measurement. Samples had been subjected to no more than two freeze-thaw cycles prior to testing; PLAC Activity has been shown to be stable over up to 12 such cycles.

Lp-PLA2 Activity Measurement.

With the exception of the real-time sample stability study, all measurements presented here were made with the PLAC Test for Lp-PLA2 Activity manufactured by the Sponsor and submitted here for FDA pre-marketing clearance; no research use Lp-PLA2 assays were used in any study discussed in this clinical performance section.

Sample and Data Availability.

All available samples were tested and are reported here. Statistical tests of the representativeness of the randomly selected sample to the complete Indicated REGARDS Population were also performed (see Section 4.6.1 Clinical Characteristics below).

Data Management.

In all studies of the Clinical Program, both subjects and treating clinicians were blinded to PLAC Activity values when providing specimens and during subsequent longitudinal follow-up (if any). Sponsor testing personnel were similarly blinded to the baseline clinical demographics and outcomes of the subjects in the Clinical Program, being provided only numerically coded samples without subject data.

For the Clinical Validation Study, all conduct and management of the REGARDS epidemiologic study, subject baseline and outcome datasets, and the archive and retention of subject specimens are the responsibility of the REGARDS Study Group, which was also blinded to PLAC Activity values in the selection and provision of samples. Test results and data exchange for the Clinical Validation Study occurred under a data management protocol using a third-party coordinator, with this analysis performed by a third-party statistician. Sponsor remains blinded to line-wise subject data for this study at the date of this filing.

For the purposes of this filing, the third party data coordinator will independently provide a line-wise subject level data file of all variables used in the Clinical Validation Study analyses directly to the FDA upon the agency's request, utilizing appropriate controls so as to preserve Sponsor blinding. Such line-wise data file requests should be directed to the Sponsor, which will authorize this direct transfer to the FDA so as to remain blinded to the actual combined clinical dataset used in the Clinical Validation Study.

All other Clinical Program data available used by the Sponsor in these analyses is provided with this filing, including separate data files with anonymized “line-wise” subject level data for the Normal Reference Study. Such data was provided directly by the sampling investigators for each such study together with the subject specimens used. Sponsor prepared and analyzed the Normal Reference Study directly, breaking the subject data blind only after testing was complete.

Statistical Analysis Plan

Purpose: This Statistical Analysis Plan (“SAP”) describes the methods to be implemented during the statistical analysis of data collected and results reported for the Clinical Investigation of the PLAC® Test for Lp-PLA₂ Activity as an Aid in Risk Prediction of Coronary Heart Disease in Patients with No Prior History of Cardiovascular Disease.

Objective: The primary objective of the study is to validate the clinical performance of the PLAC Test for Lp-PLA2 Activity as an aid in the prediction of risk for CHD events (Primary Clinical Endpoint) in a population of subjects with no prior history of cardiovascular disease (Indicated REGARDS Population).

Definitions. “Primary Clinical Endpoint” means the composite coronary heart disease (CHD) events including myocardial infarction, coronary revascularization procedures and CHD related death. “Indicated REGARDS Population” means REGARDS subpopulation having no prior history of cardiovascular events (CHD or stroke) at baseline.

Study Design.

Overall Design.

The validation study will use a case-cohort study design (Prentice 1986), sampling subjects from the REGARDS study, a large longitudinal observational study of cardiovascular diseases as described in section 9 of the protocol. A cohort random sample (CRS) selected from the REGARDS subpopulation of participants having no prior history of cardiovascular events (CHD or stroke) at baseline, referred to as the “Indicated REGARDS Population” or the “full cohort” within this protocol and SAP is combined with the Primary Clinical Endpoint cases. The combination of the CRS and cases comprises the complete validation subject set, referred to generally as the “validation case-cohort” in the following discussions.

Clinical Endpoints.

The primary outcome to be assessed is a composite endpoint of total CHD events (the “Primary Clinical Endpoint”). A Primary Clinical Endpoint event (hereafter simply referred to as an “event”) will be defined as (a) myocardial infarction, (b) coronary revascularization, or (c) CHD-related death. Any component or other endpoints (“Secondary Clinical Endpoints”) and their related events will be specifically identified as such in the following discussion and reports.

Expression of Lp-PLA2 Activity Results.

Lp-PLA2 activity will be expressed as a binary categorical variable for efficacy analyses with individual study subject results dichotomized using a pre-specified analysis cut point of 225 nmol/min/mL. Subjects with values below the cut point will be considered at reduced risk for events (the “low Lp-PLA2 group”). Conversely, subjects with values greater than or equal to the cut point will be considered at increased risk for events (the “high Lp-PLA2 group”).

Subsequent to the binary efficacy analyses, the underlying continuous Lp-PLA2 activity results may also be used in the additional analyses to the extent needed to achieve a well-powered analysis (see Sample Size and Power Assessment below). In such continuous analysis, the Lp-PLA2 activity results distribution will be assessed for normality, and log transformation (log 2) performed if required. The resulting transformed values are used in parallel in such analyses as noted below.

Analysis Sets.

The protocol will employ three different analysis sets. In addition to the full study analysis set, two racial subpopulations will form two non-overlapping analysis sets.

FAS: The full analysis set consists of the CRS and remaining added Primary Clinical Endpoint cases.

WAS: The Caucasian subpopulation analysis set.

BAS: The African-American subpopulation analysis set.

The use of the different analysis sets to evaluate the efficacy of the marker and the racial differences in marker performance is subject to the results from a statistical interaction test as detailed below.

Statistical Methods.

Validation Case-Cohort Study Subject Weights.

Adjustments to the partial likelihood are required because cases are over-represented in the validation case-cohort compared to the full cohort. As such, estimates of incidence rates from un-weighted analyses would not be representative of disease incidence in the full cohort. The case-cohort weighting method of Barlow (Barlow 1994) will be used for the validation case-cohort study given its consistency with the other validation study methods used versus other published weighting methods reviewed (Barlow et al. 1999).

The Barlow case-cohort weighting method is an inverse probability weighting (“IPW”) methodology for case-cohort study design, and builds upon earlier, more generalized work on sampling weights in survey and study design (Horvitz and Thompson 1952; Gelman and Carlin 2001). This method relies upon applying a further weighting factor to the CRS sub-cohort which is calculated as the ratio of the full cohort to the CRS sub-cohort. The method weights each CRS sub-cohort member by the inverse of the CRS sampling probability (or fraction) within the full cohort. The method allows CRS controls to be “up-weighted” back to the full cohort population from which they were chosen. Since all cases are utilized, the subject weight adjustment for cases is one and remains at the same base weight as in a full cohort analysis (this is true for both CRS cases and the remaining cases added to the CRS to arrive at the validation case-cohort study).

The case-cohort IPW weights will be used in all cross-sectional and longitudinal analyses to generate descriptive statistics, absolute risk estimates from Kaplan-Meier analyses, and Cox proportional hazards regression estimates.

General Statistical Methods.

All summaries and analyses will be presented in tabular or graphical form. Analyses will utilize the IPW case-cohort weights mentioned above as appropriate, with weighting approach identified in each such analysis output. All statistical tests will be 2-tailed and performed at the 5% significance level, unless stated otherwise.

“Descriptive statistics” refers to mean, standard deviation (SD), minimum, 25th, 50th (median), 75th percentiles and maximum values for continuous measurements, and number (count) and percentage of subjects within each level of a categorical measurement, and for each composite or component endpoint described. The sample size and number of missing observations will also be noted for each measurement.

Other specific statistical methods to be used will be pre-specified below as required for each analysis and defined in the final report. Statistical models and methods will also be evaluated for their appropriateness in each primary and secondary analysis (e.g. Cox proportional hazards models assessed for violation of the proportional hazards assumption, and continuous measurements for normality). In the event of a model violation, alternative statistical methods (e.g. extended Cox models, value transformations or categorizations) and alternative methods justification will be included in the final statistical report.

Missing Data.

Prior to database lock, the data will be reviewed for accuracy and completeness. It is expected that there will be very little missing data. Missing values will not be imputed for Lp-PLA2 activity testing results in any validation study analysis or for any other study variable in the primary and secondary efficacy analyses.

Imputation of other variables (non-Lp-PLA2 activity results such as model covariates) will be performed only (i) where needed to maximize available events and achieve a well-powered analysis (see Sample Size and Power Assessment below) and (ii) where missing values represent less than 10% of total validation case-cohort study values for such imputed variable. To the extent such imputation is required, multiple imputation with additive regression and predictive mean matching using the remaining full covariate data will be employed in each such analysis (Little 2004; van Buuren et al. 2006; de Groot et al. 2008; Siddique 2008), potentially including such variants as Markov Chain Monte Carlo. The validity of any imputations will be confirmed and tested for evidence of bias by performance of the same analysis solely within the subset of subjects with complete data.

Multiple Comparisons/Multiplicity.

Initially, a single hypothesis test on the race by Lp-PLA2 activity group interaction will be evaluated at a 5% significance level in the FAS (p<0.05).

If the interaction fails to achieve this level of significance, the interaction term will be removed from the model and the prognostic efficacy analysis for the Lp-PLA2 group effect will be evaluated in the FAS at the 5% level, followed by tests in the individual race subgroups as supportive descriptive analyses. In this event, no adjustment for multiplicity will be made in the supportive evaluation of the WAS and BAS individually.

If the interaction is significant, the subsequent efficacy analyses will be performed on the WAS and BAS, followed by tests in the overall study FAS (including an interaction term as appropriate) as a supportive descriptive analysis. In this scenario, any race subgroup demonstrating a statistically significant Lp-PLA2 group effect would be considered a positive study, and the individual races will each be evaluated at a 5% significance level (p<0.05) given the significant result of the interaction test.

The analyses will be performed and results presented for each of the individual analysis sets (FAS, WAS, and BAS) regardless of their position as primary efficacy or supportive analyses per the above discussion of multiplicity adjustments,

The order of importance of the secondary endpoints will be the order in which they are presented in the Efficacy Analyses section. The second hypothesis will be tested only if the first alternative (null) hypothesis has been rejected. Each subsequent alternative hypothesis will be tested only if the previous alternative hypotheses have been rejected, until the last alternative hypothesis has been reached or one of the alternative hypotheses is not rejected, thus maintaining the overall significance level at 5%. The remaining additional analyses are considered supportive or descriptive of the main efficacy analyses and no multiplicity considerations will be attempted.

Sample Size and Power Assessment.

As these are baseline samples from an existing enrolled study, the REGARDS parent cohort sample size was not pre-determined based on the Primary Clinical Endpoint of this Lp-PLA2 activity validation study. However, a sample size assessment, based on the actual events that have occurred to the latest REGARDS adjudication date of Dec. 31, 2010 (the study is continuing to follow its participants), is presented here in order to provide an assessment of the study statistical power for the validation study analyses by FAS, WAS, and BAS.

The various analyses of the validation study are considered statistically well-powered if they are estimated to have at least 90 percent power (1−β≧0.90) to detect a pre-specified relative risk or proportional hazard ratio for total events at a two-sided significance level of 5 percent (?=0.05). In the Cox proportional hazards analyses, covariate correlation with Lp-PLA2 is assessed at a variance inflation factor (VIF) ranging from 1.0 (R2=0.0) in univariate analyses to 2.0 (R2=0.5) in extensively risk-adjusted analyses using continuous variables (Hsieh & Lavori 2000; Schoenfeld 1983). Power is driven by predictor performance assumptions from prior studies of Lp-PLA2 activity, specifically (i) reduced hazard ratio estimates with increasing model size and (ii) increased Lp-PLA2 activity value correlation with the addition of lipoprotein covariates, due to the Lp-PLA2 enzyme's known molecular association and biological function in cholesterol and phospholipid metabolism.

In the binary categorization power analyses below, it is assumed that the Lp-PLA2 activity analysis cut point of 225 nmol/min/mL divides the weighted validation case-cohort FAS study population at its 70th percentile, with the lower 70 percent subpopulation formed representing the low Lp-PLA2 group and the higher 30 percent subpopulation formed representing the high Lp-PLA2 group, respectively. In the WAS and BAS populations, respectively, divisions at the 62nd and 83rd percentiles are assumed based value distributions in prior studies.

In the continuous Lp-PLA2 activity power analyses, lowered hazard ratios (versus those achieved using an optimized binary cut point) are also assumed consistent with prior studies. In addition to the stated hazard ratio and VIF assumptions, all power analyses included the impact of the case-cohort design and presented CRS size (Cai and Zeng 2004; Kim et al. 2006).

An analysis of power for each endpoint using the actual total Primary Clinical Endpoint cases within each study set is presented below. There are total cases of 1,001 (FAS), 639 (WAS), and 362 (BAS), respectively. For the primary and secondary analyses, the minimum detectable hazard ratio at a 90 percent power (1−β≧0.90) is given; the expected univariate relative risk and hazard ratios are 2.0 based on past studies (the clinical implications of such expected hazard ratios is discussed further elsewhere in the clinical validation plan).

The minimum total cases required to achieve a 0.9 and 0.8 power to detect the expected hazard ratio and VIF level is also provided below for all Cox proportional hazards regression models, which is the primary methodology used in the other analyses. By design, these other analyses, which include subgroups of the Indicated REGARDS Population and component events fully contained within the Primary Clinical Endpoint analyses, generally contain a lesser number of subjects, cases and events than is contained within the full cohort or the primary efficacy endpoint.

Kaplan-Meier Logrank Test (Primary).

The existing total events in each of the validation case-cohort study sets are well-powered (1−β≧0.90) to detect a minimum relative risk ratio of 1.6 using Kaplan-Meier estimators of absolute risk and a logrank test of the differences (r₁≠r₂, α=0.05) between the two subpopulations formed through a binary categorization by Lp-PLA2 activity values assuming the above analysis cut point population splits. No variance inflation factor is applied in this univariate analysis.

Univariate Cox Proportional Hazards Regression (Secondary).

The existing total events in each of the validation case-cohort study sets are well-powered (1−β≧0.90) to detect a minimum hazard ratio of 1.6 using Cox proportional hazards regression model analysis (HR>1.0, α=0.05) between the two subpopulations formed through a binary categorization by Lp-PLA2 activity values assuming the above analysis cut point population splits. No variance inflation factor is applied in this univariate analysis.

Multivariate Cox Proportional Hazards Regression (Binary).

Three model sizes using multivariate Cox proportional hazards regression of Lp-PLA2 activity binary categorization are planned in the additional analyses with the resulting power analyses (see Multivariate Cox Proportional Hazard Models below) in the validation case-cohort study summarized for each increasing model size in Table, below. All analyses assume Cox proportional hazards regression model analysis (HR>1.0, α=0.05) between the two subpopulations formed through a binary categorization by Lp-PLA2 activity values assuming the above analysis cut point population splits. Expected hazard ratios and variance inflation factors are shown, based on prior studies.

TABLE

| | | | 70/30 Split | | 62/38 Split | | 83/17 Split | |
|---|---|---|---|---|---|---|---|---|
| | | | (FAS) Cases | | (WAS) Cases | | (BAS) Cases | |
| | Expected | | Required at | | Required at | | Required at | |
| Cox | Performance | | Power (1-β) of | | Power (1-β) of | | Power (1-β) of | |
| Model | HR | VIF | 0.80 | 0.90 | 0.80 | 0.90 | 0.80 | 0.90 |
| Demographically- | 1.70 | 1.25 | 166 | 223 | 148 | 198 | 247 | 331 |
| Adjusted | | | | | | | | |
| Risk-Adjusted | 1.50 | 1.25 | 285 | 381 | 254 | 340 | 423 | 567 |
| Extensively Risk- | 1.30 | 1.67 | 905 | 1,212 | 807 | 1,080 | 1,347 | 1,804 |
| Adjusted | | | | | | | | |

As noted above, the first demographically-adjusted model appears well-powered for the Primary Clinical Endpoint in all three analysis sets (FAS, WAS, and BAS). The second risk-adjusted model appears well-powered in the FAS and WAS, but under-powered in the BAS. The third extensively risk-adjusted model, including several covariates and lipoproteins known to be correlated with Lp-PLA₂ activity group (multiple regression R²=0.40 respectively), is under-powered with the existing total cases in all sets. Given the above assumptions, the full validation case-cohort study (FAS) is estimated to only have a power of 0.8 for this third extensively risk-adjusted analysis.

Multivariate Cox Proportional Hazards Regression (Continuous).

Modeling of continuous standardized Lp-PLA2 activity values is also specified as an option within the additional analyses in order to preserve study power, particularly in the extensively risk-adjusted Cox models, subgroup, interaction tests, and component endpoint analyses. Use of continuous biomarker values can frequently increase the statistical power to identify prognostic biomarkers, although this is often offset by reductions in their hazard ratios, as the relationships of continuous biomarkers to prediction are frequently diluted across their full reported ranges (versus when they are optimized at a single discrete value cut point in binary categorizations), and further risks potential increases in biomarker correlations with covariates.

This power improvement is demonstrated using the same three multivariate Cox proportional hazards regression models against continuous Lp-PLA2 activity, with the resulting power analyses in the validation case-cohort study summarized in Table below. All analyses assume Cox proportional hazards regression model analysis (HR>1.0, α=0.05) against continuous standardized Lp-PLA2 activity values (mean=0, standard deviation=1), where the Cox hazard ratio for the marker indicates the increase in proportional hazards per one standard deviation movement in Lp-PLA2 activity. Expected hazard ratios and variance inflation factors are also shown, based on prior studies.

TABLE

| | Expected | | (FAS, WAS, BAS) | |
|---|---|---|---|---|
| | Performance | | Cases Required at Power (1 - β) of | |
| Model | HR | VIF | 0.80 | 0.90 |
| Univariate | 1.50 | 1.00 | 48 | 64 |
| Demographically- | 1.30 | 1.25 | 143 | 191 |
| Adjusted | | | | |
| Risk-Adjusted | 1.25 | 1.43 | 226 | 302 |
| Extensively | 1.20 | 2.00 | 473 | 633 |
| Risk-Adjusted | | | | |

With the existing total events in the validation case-cohort study FAS (1,001 cases) and WAS (639 cases) study sets, all Cox proportional regression models are shown to be well-powered to detect the expected hazard ratios, at assumed VIF levels when using continuous Lp-PLA2 activity values. The BAS study set (362 cases) is underpowered in the third extensively risk-adjusted Cox model analysis.

Continuous measures may also enable better elucidation of the relationship of risk with Lp-PLA2 activity in other extensively risk-adjusted, subgroup, interaction, and component event endpoint analyses undertaken as additional analyses.

Demographics and Baseline Characteristics.

Demographic and baseline clinical variables will be summarized with descriptive statistics for the FAS, WAS and BAS by study primary endpoint outcome (cases, controls) and for the complete CRS population within each analysis set. Additional subgroups defined by the baseline categorical covariates (gender, diabetes, hypertension, current smoking, HDL, LDL, and Lp-PLA2 activity risk categories, as further described below) will also be provided.

A complete listing of quantitative, discrete, derived and Lp-PLA2 activity marker analysis variables used in the validation study, their statistical programming code approach, and use of each in the individual study analyses include:

| Variable Name | Type | | | Cox | | |
|---|---|---|---|---|---|---|
| (Abbreviation) - units | (Levels) | Values | Descriptives | Models | Subgroups | Interactions |
| I. Quantitative | | | | | | |
| Variables | | | | | | |
| Age (Age) - Years | Quantitative | Continuous | X | a, b, c | See Age-C | X |
| Blood Pressure, Systolic | Quantitative | Continuous | X | See HTN | See HTN | |
| (SBP) - mmHg | | | | | | |
| Blood Pressure, Diastolic | Quantitative | Continuous | X | See HTN | See HTN | |
| (DBP) - mmHg | | | | | | |
| Glucose, Fasting (Glu) - | Quantitative | Continuous | X | See DM | See DM | |
| mg/dL | | | | | | |
| High-Density Lipoprotein | Quantitative | Continuous | X | See | See HDL-C | |
| (HDL) - mg/dL | | | | HDL-C | | |
| Low-Density Lipoprotein | Quantitative | Continuous | X | See | See LDL-C | |
| (LDL) - | | | | LDL-C | | |
| mg/dL | | | | | | |
| Total Cholesterol (TC) - | Quantitative | Continuous | X | | | |
| mg/dL | | | | | | |
| II. Discrete Variables | | | | | | |
| Diabetes (DM) | Factor (2) | Yes, No (baseline) | X | b, c | Covariate | X |
| Gender (Gender) | Factor (2) | Male, Female | X | a, b, c | X | X |
| | | (baseline) | | | | |
| Race (Race) | Factor (2) | Caucasian (baseline), | X | a, b, c | X | X |
| | | African-American | | | | |
| Hypertension (HTN) | Factor (2) | Yes, No (baseline) | X | b, c | Covariate | X |
| Current Smoking | Factor (2) | Yes, No (baseline) | X | b, c | Covariate | X |
| (Smoke) | | | | | | |
| III. Derived Variables | | | | | | |
| Age Category (AGE-C) | Factor (2) | 45-64 (baseline), | | | X | |
| | | ≧65 | | | | |
| Age Decade (AGE-D) | Factor (5) | 45-54 (baseline), 55-64, | X | | | |
| | | 65-74, 75-84, ≧85 | | | | |
| Framingham Risk Score | Quantitative | Continuous | X | | | |
| (FRS) | | (REGARDS Calculated) | | | | |
| Framingham Risk | Factor (3) | High, Medium, Low | X | | | |
| Category (FRS-C) | | (baseline) | | | | |
| HDL Category (HDL-C) | Factor (2) | <40 mg/dL (M) or <50 mg/dL | X | c | Covariate | X |
| | | (F), baseline | | | | |
| LDL Category (LDL-C) | Factor (2) | ≧130 mg/dL, | X | c | Covariate | X |
| | | <130 mg/dL (baseline) | | | | |
| IV. Lp-PLA₂ Activity | | | | | | |
| Variables | | | | | | |
| Lp-PLA₂ Activity | Quantitative | Continuous | X | See | See below | |
| (PLACA-C) - nmol/min/mL | | | | below | | |
| Lp-PLA₂ Activity Risk | Factor (2) | ≧225 nmol/min/mL, | X | X | X | X |
| Category (PLACA-C) | | <225 nmol/min/mL | | | | |
| | | (baseline) | | | | |
| Lp-PLA₂ Activity Tertile | Factor (3) | 1st (baseline), 2nd, 3rd | X | | | |
| (PLACA-T) | | (Calculated) | | | | |
| Lp-PLA₂ Activity Log₂ | Quantitative | Continuous | X | ◯ | | ◯ |
| Transformed | | (Calculated) | | | | |
| Z-Score (PLACA-Z) | | | | | | |

Footnotes
a = Demographically-Adjusted Cox Model.
b = Risk-Adjusted Cox Model.
c = Extensively Risk-Adjusted Cox Model.
X = Used in the analysis indicated in the respective column.
◯ = Option to use in analysis or used under certain conditions (continuous biomarker results)

Quantitative continuous measures include age, systolic and diastolic blood pressures, total cholesterol, LDL, HDL, and fasting glucose.

Discrete categorical measures include gender, race, hypertension, diabetes, and current smoking status.

Derived variables (those calculated from one or more of the preceding measures) include both quantitative continuous measures and discrete categorical variables, such as Framingham Risk Score and Framingham Risk Category (low, intermediate, high), age decade (45-54, 55-64, 65-74, 75-84, and ≧85 years old), LDL category (130 mg/dL cut point), and HDL category (with gender specific cut points at 40 mg/dL for males and 50 mg/dL for females).

In addition, certain discrete variables will be used to define pre-specified subgroups or subpopulations within the validation case-cohort study used to describe the range of continuous Lp-PLA2 activity values reported. Lp-PLA2 activity values will be summarized in these subgroups, in the binary categorization risk categories formed by the analysis cut point, and using various percentile divisions (such as tertiles and quartiles), using the same metrics as other continuous variables, and differences among the subpopulations will be assessed comparing that category to the rest of the population. Differences in distributions of demographic and clinical variables between subgroups will also be analyzed using statistical methods confirmed during statistical methods development and programming validation (Kruskal-Wallis or a Wilcoxon Rank Sum test for continuous variables depending on the number of groups being compared, and Fisher's Exact tests for categorical variables).

Following the efficacy analyses, the same demographic and clinical variables will be summarized with descriptive statistics for the cases and non-case “control” subjects. This analysis will be repeated for case subgroups experiencing each of the component and composite Secondary Clinical Endpoint events contained within the Primary Clinical Endpoint and examined in the additional component analysis. Within the total cases the distributions of fatal and non-fatal events and informing sources will be summarized.

Efficacy Analyses.

In the evaluation of a prognostic biomarker, the most relevant statistical methods for determination of efficacy are Kaplan-Meier survival analyses, including their associated absolute risk estimates, and Cox proportional hazards regression models. As previously noted, each of these analyses will be conducted using methods compatible with subject weights, using the validation case-cohort weights unless specifically noted otherwise, for the Primary Clinical Endpoint cases and controls. Also as noted above, Cox proportional hazards models will be assessed for violation of the proportional hazards assumption in each such primary and secondary analysis.

Kaplan-Meier and Absolute Risk (Primary).

The primary hypothesis is that there is a statistically significant difference in the absolute risk for Primary Clinical Endpoint events between the low Lp-PLA₂ group (r₁) and the high Lp-PLA₂ group (r₂) (H_{A}: r₁≠r₂), measured over the full available validation study duration. The null hypothesis is that both groups share the same absolute risk for events (H₀: r₁=r₂).

Initial Analysis: Lp-PLA₂ Group by Race Interaction Test.

A Cox proportional hazards regression model will be used to test the Lp-PLA₂ group by race interaction on the FAS at a significance level of 0.05 (p<0.05). If the interaction term is not significant and performance results appear similar across races, then races can be pooled.

Efficacy Analysis.

A weighted Kaplan-Meier analysis (Kaplan & Meier, 1958) and plot of the event-free survival probabilities in both the low Lp-PLA₂ group and the high Lp-PLA₂ group of the appropriate population(s) will be constructed with right-censoring of the non-case controls at their latest follow-up date in order to test the primary hypothesis.

The statistical test performed will be a logrank test (Mantel, 1966) at the above stated significance levels (p<0.05 as noted previously) utilizing the full available study duration. The analysis will be presented in each of the FAS, the BAS, and the WAS individually regardless of the results of the interaction test.

The survival probabilities at 1, 3, 5, and 7 years and absolute risks of total events will be estimated in each group within each of the FAS, BAS, and WAS, using the weighted Kaplan-Meier analysis described above over the full available study duration, and presented together with each associated 2-sided 95% Confidence Interval (95% CI).

Univariate Cox Proportional Hazards Model (Secondary).

A Cox proportional hazards regression model will be employed in each such population to estimate the relative risk for total events in the high Lp-PLA₂ group relative to the low Lp-PLA₂ group, as estimated by the hazard ratio (HR) derived from the model's beta coefficient (H_{A}: HR>1.0). The null hypothesis is that the low Lp-PLA₂ group and the high Lp-PLA₂ group have the same relative risk (H₀: HR=1.0).

Using the appropriate population, a univariate weighted Cox regression will be performed with the binary Lp-PLA₂ categorization into the high and low Lp-PLA₂ groups as the sole term in the model. Regression coefficient, standard error (SE), hazard ratio (HR) with associated 95% CI, and p-value will be reported.

High Lp-PLA₂ will be determined to be a significant predictor of events if (a) the point estimate of the HR exceeds 1.0 and (b) the HR 95% CI does not overlap 1.0.

Additional Analyses

Parallel Analyses; Interaction Terms; Continuous Lp-PLA2 Models.

The following analyses are considered supportive and/or descriptive of the primary and secondary efficacy results and are not adjusted for multiplicity; they will be performed in parallel in the FAS, WAS and BAS. Race will be included as a covariate when the analysis is performed in the FAS and eliminated as a covariate when analysis is performed in the WAS or BAS separately (with an additional parallel FAS analysis with a Lp-PLA₂ group by race interaction term added if appropriate based on the preceding analysis). Parallel analyses using continuous Lp-PLA₂ activity results will also be performed as described above as needed to improve power (§5.5 Sample Size and Power Assessment) and also in univariate continuous Lp-PLA₂ activity analyses following the previous Cox efficacy analyses in order to gain a baseline continuous hazard ratio for comparability.

Multivariate Cox Proportional Hazard Models.

Three additional multivariate Cox proportional hazards regression models will be performed to further describe the risk-adjusted predictive power of Lp-PLA₂ group categorization for total events, expanding the previous Cox regression model of the binary Lp-PLA₂ categorization into the high and low Lp-PLA₂ groups Univariate Cox Proportional Hazards Model (Secondary) above), by controlling for other covariates of event risk. For each term in each of the three models, regression coefficient, SE, HR and its associated 95% CI, and p-value will be reported.

High Lp-PLA₂ will be determined to be an independent predictor of events if (a) the Lp-PLA₂ term point estimate of the HR exceeds 1.0, and (b) the HR 95% CI does not overlap 1.0.

The following multivariate Cox proportional hazards regression models will be used:

Demographically-Adjusted Cox Model.

A first “demographically-adjusted model” will adjust predictive power by including the covariates of age (continuous), race (Caucasian/African-American), and gender (male/female) with binary Lp-PLA2 category. These represent demographic variables controlled for in the REGARDS parent study design.

Risk-Adjusted Cox Model.

A second “risk-adjusted model” will additionally adjust for diabetes (yes/no), hypertension (yes/no), and current smoking (yes/no). These added factors represent the most common primary prevention risk markers which are not directly associated with the lipoprotein and lipid metabolism related to Lp-PLA2 activity.

Extensively Risk-Adjusted Cox Model.

A third “extensively risk-adjusted model” will add high-density lipoprotein (HDL, expressed as a binary variable with cut point=40 mg/dL for males and 50 mg/dL for females) and low-density lipoprotein (LDL, expressed as a binary variable with cut point at 130 mg/dL), utilizing cut points published in public health guidelines for treatment in lower risk categories (NCEP, 2001 and 2004). These added primary prevention risk markers are known from past studies to be structurally associated with the Lp-PLA2 enzyme, which has a biological mechanism related to both of these lipoprotein types (Lp-PLA2 Studies Consortium, 2010). As a result, their addition to Lp-PLA2 may represent an overcorrection in multivariate regression modeling, which will be explored using further statistical procedures, including examining more parsimonious models, as appropriate.

Subgroup Analyses.

The absolute risk estimates and univariate and multivariate Cox models will be repeated individually within each gender subgroup (and any other pre-specified subgroups shown in Appendix B) for the Lp-PLA₂ categorization binomial variable using the above (i) univariate, (ii) demographically-adjusted and (iii) extensively risk-adjusted Cox models (adjusted appropriately for the subgroup under analysis).

The regression coefficient, SE, HR and its associated 95% CI, and p-value will be reported for the Lp-PLA₂ categorization binomial variable within each subgroup, and compared between the two subgroups (e.g., between males and females, Caucasians and African Americans). Given the under-powering of the subgroups, this process and comparison will also be repeated for each of the three models substituting Lp-PLA₂ activity as a standardized continuous variable, reporting the same statistics and comparison test across models representing the paired subgroups.

The completed subgroup analysis will result in a total of six model comparisons, three using binomial Lp-PLA₂ activity and three using continuous Lp-PLA₂ activity as predictive variables at different model sizes, across each of the two subgroups represented within a comparison.

Secondary Clinical Endpoint Analyses.

The primary and secondary analyses, including absolute risk estimates and Cox models, will be repeated as additional analyses for the individual components of the composite Primary Clinical Endpoint, i.e. separately by (a) myocardial infarction, (b) coronary revascularization, (c) CHD-related death. Each of these is referred to here as a Secondary Clinical Endpoint. The further additional analyses above may also be repeated in such components for descriptive purposes, as appropriate, in order to better describe the relationship of Lp-PLA₂ activity and event risk.

Treatment of CVD

Any of the methods described herein may also include treating the patient for cardiovascular disease or CHD when the Lp-PLA2 activity level is equal to or above the cutoff value. Any appropriate treatment may be indicated or advised based on an Lp-PLA2 activity level equal to or above the cutoff value. A treatment for CHD may be or may involve any type treatment as known in the art, such as administering a medication, using a medical device, surgery, or using another type of treatment. A treatment may include a administering a medication, such as administering an Lp-PLA2 inhibitor (Leach 2001), an anti-inflammatory agent, an anti-thrombotic agent, an anti-platelet agent, a fibrinolytic agent, a lipid reducing agent (e.g., statin), a direct thrombin inhibitor, a glycoprotein II b/IIIa receptor inhibitor, an agent that bind to cellular adhesion molecules and inhibit the ability of white blood cells to attach to such molecules (e.g. anti-cellular adhesion molecule antibodies), an aldosterone antagonist, an angiotensin-converting enzyme (ACE) inhibitor, angiotensin-receptor blockers (ARBs), aspirin, a beta blocker, digoxin, a diuretic, an inotrope, digitalis, hydralazine and nitrates, statins, and warfarin. A treatment may include a performing a surgery, such as performing a coronary bypass surgery, heart valve repair or replacement, an implantable cardioverter-defibrillator (ICD), cardiac resynchronization therapy, a heart pump, or a heart transplant. Another type of treatment may include, for example, implanting stem cells such as cardiac or other stem cells.

Anti-inflammatory agents include Alclofenac; Alclometasone Dipropionate; Algestone Acetonide; Alpha Arnylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Salycilates; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Glucocorticoids; Zomepirac Sodium.

Anti-thrombotic and/or fibrinolytic agents include Plasminogen (to plasmin via interactions of prekallikrein, kininogens, Factors XII, XIIIa, plasminogen proactivator, and tissue plasminogen activator [TPA]) Streptokinase; Urokinase: Anisoylated Plasminogen-Streptokinase Activator Complex; Pro-Urokinase; (Pro-UK); rTPA (alteplase or activase; r denotes recombinant), rPro-UK; Abbokinase; Eminase; Sreptase Anagrelide Hydrochloride; Bivalirudin; Dalteparin Sodium; Danaparoid Sodium; Dazoxiben Hydrochloride; Efegatran Sulfate; Enoxaparin Sodium; lfetroban; Ifetroban Sodium; Tinzaparin Sodium; retaplase; Trifenagrel; Warfarin; Dextrans.

Anti-platelet agents include Clopridogrel; Sulfinpyrazone; Aspirin; Dipyridamole; Clofibrate; Pyridinol Carbamate; PGE; Glucagon; Antiserotonin drugs; Caffeine; Theophyllin Pentoxifyllin; Ticlopidine; Anagrelide.

Statins are important drugs used to lower cholesterol and to prevent heart disease. These drugs include lovastatin (Mevacor), pravastatin (Pravachol), simvastatin (Zocor), fluvastatin (Lescol), atorvastatin (Lipitor), and rosuvastatin (Crestor). In 2007, the Food and Drug Administration (FDA) approved atorvastatin to reduce the risks for hospitalization for heart failure in patients with heart disease.

Direct thrombin inhibitors include hirudin, hirugen, hirulog, agatroban, PPACK, thrombin aptamers. Glycoprotein IIb/IIIa receptor Inhibitors are both antibodies and non-antibodies, and include but are not limited to ReoPro (abcixamab), lamifiban, tirofiban. One preferred agent is aspirin. Aspirin is a type of non-steroid anti-inflammatory (NSAID). Aspirin is recommended for preventing death in patients with heart disease, and can safely be used with ACE inhibitors, particularly when it is taken in lower dosages (75-81 mg).

In particular, the techniques described herein may be used to treat a subject by providing aspirin (e.g., acetylsalicylic acid) when the subject's level of Lp-PLA2 exceeds a cutoff alone or in combination with one or more other biomarkers. Curiously, previous work has taught away from the use of aspirin when the level of Lp-PLA2 is above normal in a patient. See, e.g., Hatoum et al. “Dietary, lifestyle, and clinical predictors of lipoprotein-associated phospholipase A2 activity in individuals without coronary artery disease” in Am J Clin Nutr 2010; 91:786-93. (“Aspirin use was also positively associated with Lp-PLA2 activity”).

Warfarin (Coumadin) is generally recommended only for patients with heart failure who also have: atrial fibrillation, a history of blood clots to the lungs, stroke, or transient ischemic attack, a blood clot in one of their heart chambers. Other drugs that may be used may include Nesiritide (Natrecor), Erythropoietin, Tolvaptan, Levosimendan, etc.

Angiotensin-converting enzyme (ACE) inhibitors are often used for treating patients with heart failure. ACE inhibitors open blood vessels and decrease the workload of the heart. They are used to treat high blood pressure but can also help improve heart and lung muscle function. ACE inhibitors are particularly important for patients with diabetes, because they also help slow progression of kidney disease.

Angiotensin-Receptor Blockers (ARBs), also known as angiotensin II receptor antagonists, are similar to ACE inhibitors in their ability to open blood vessels and lower blood pressure. They may have fewer or less-severe side effects than ACE inhibitors, especially coughing, and are sometimes prescribed as an alternative to ACE inhibitors. Some patients with heart failure take an ACE inhibitor along with an ARB.

Beta blockers are almost always used in combination with other drugs, such as ACE inhibitors and diuretics. They help slow heart rate and lower blood pressure. When used properly, beta blockers can reduce the risk of death or re-hospitalization. Beta blockers can lower HDL (“good”) cholesterol, so have not previously been used with patients having a high level of Lp-PLA2.

Diuretics cause the kidneys to rid the body of excess salt and water. Fluid retention is a major symptom of heart failure. Aggressive use of diuretics can help eliminate excess body fluids, while reducing hospitalizations and improving exercise capacity. These drugs are also important to help prevent heart failure in patients with high blood pressure. In addition, certain diuretics, notably spironolactone (Aldactone), block aldosterone, a hormone involved in heart failure. This drug class is beneficial for patients with more severe heart failure (Stages C and D). Patients taking diuretics usually take a daily dose. Diuretics, or any of the treatments described herein, may be modified based on the level of Lp-PLA2 or Lp-PLA2 in combination with one or more other biomarkers. For example, the amount and timing of the diuretic (or other heart failure agent) may be adjusted on this basis.

Aldosterone is a hormone that is critical in controlling the body's balance of salt and water. Excessive levels may play important roles in hypertension and heart failure. Drugs that block aldosterone are prescribed for some patients with symptomatic heart failure. They have been found to reduce mortality or death rates for patients with heart failure and coronary artery disease, especially after a heart attack. These blockers pose some risk for high potassium levels.

Digitalis is derived from the foxglove plant. It has been used to treat heart disease since the 1700s. Digoxin (Lanoxin) is the most commonly prescribed digitalis preparation. Digoxin decreases heart size and reduces certain heart rhythm disturbances (arrhythmias). Unfortunately, digitalis does not reduce mortality rates, although it does reduce hospitalizations and worsening of heart failure. Controversy has been ongoing for more than 100 years over whether the benefits of digitalis outweigh its risks and adverse effects. Digitalis may be useful for select patients with left-ventricular systolic dysfunction who do not respond to other drugs (diuretics, ACE inhibitors). It may also be used for patients who have atrial fibrillation.

Hydralazine and nitrates are two older drugs that help relax arteries and veins, thereby reducing the heart's workload and allowing more blood to reach the tissues. They are used primarily for patients who are unable to tolerate ACE inhibitors and angiotensin receptor blockers. In 2005, the FDA approved BiDil, a drug that combines isosorbide dinitrate and hydralazine. BiDil is approved to specifically treat heart failure in African-American patients.

### Example 1

### Establishment of Clinical Cut Point for LpPLA2 Activity

Purpose: The selection of the clinical cut point and definition of the value to be used for clinical evaluation of Lp-PLA2 activity for the prediction of risk for coronary heart disease (CHD) in subjects with no prior history of cardiovascular disease.

Scope: The cut point described in this study was established for the PLAC Test for Lp-PLA2 Activity and provides justification for the cut point to be used in future clinical validation studies for risk prediction of coronary heart disease in patients with no prior history of cardiovascular disease.

Background: A number of large population-based studies have demonstrated that elevated serum or plasma levels of Lp-PLA2 activity are associated with increased risk for incident CHD (Packard et al. 2006; Ballantyne et al. 2004; Ballantyne et al. 2005; Oei et al 2005; Blankenberg et al. 2003; Thompson et al. 2010). Actual values for each study cited vary and are not directly comparable to PLAC Test for Lp-PLA2 Activity due to the varied test formats and methods used to measure Lp-PLA2 activity. In the four methods utilized in these referenced studies, none of the assays implemented calibration and none of the assays were cleared for IVD use. PLAC Test for Lp-PLA2 Activity is a comparatively new assay; therefore published studies with this kit format are limited. PLAC Test is the only Lp-PLA2 activity assay that utilizes calibration.

Two analytical tools commonly used for risk prediction are Kaplan Meier survival analysis, describing the event-free survival rates observed over time in a population (absolute risk of events=one minus the event-free survival rate), and the Cox proportional hazards model describing the potential effect of one or more variables on time to event (hazard ratio). In Kaplan-Meier analysis, a log-rank test is utilized to establish statistical differences in the survival rates between different populations. In Cox proportional hazards models using categorical variables, the time to event for a population category defined by such a variable is expressed as a proportional hazards ratio relative to the baseline hazard function (the remaining population not categorized for the variable).

In most publications, baseline Lp-PLA2 activity values from populations were generally analyzed for risk prediction by Cox proportional hazard ratios based on either continuous, standardized, tertile or quartile thresholds of the cohort distributions, rather than single threshold values. Positive clinical signals were observed using the upper tertile or upper quartile cut points in these studies. JUPITER described below is an independent study testing the association of PLAC Test for Lp-PLA2 Activity and risk prediction for coronary heart disease.

Selection of Proposed Cut Point

JUPITER: Justification for the Use of Statins in Prevention: an Intervention Trial Evaluating Rosuvastatin

Lp-PLA2 activity was measured by the PLAC Test for Lp-PLA2 Activity in JUPITER, a contemporary international study including US subjects, with blood collected in 2003-2006. Lp-PLA2 Activity was measured in frozen plasma samples drawn from apparently healthy subjects with no prior CVD at baseline and stored at ≦−70° C. from the placebo arm of the study. Inclusion criteria included LDL-C<130 mg/dL and CRP≧2 mg/L, and the subjects were followed for cardiovascular outcomes (non-fatal myocardial infarction, non-fatal stroke, hospitalization for unstable angina, arterial revascularization or cardiovascular death). Median follow-up time was 1.9 years with a maximum of 5 years. The median age of the subjects was 66 years and 36% were female. Lp-PLA2 activity levels measured by the PLAC Test demonstrated association with incident cardiovascular events in the placebo arm of the study (n=5,446). The investigators assessed cardiovascular risk by Cox proportional hazards models across quartiles of Lp-PLA2 activity. In analysis of subjects with levels in the highest quartile >223 nmol/min/mL (75th percentile of the trial population) vs the lowest quartile, the hazard ratio was 2.83 (CI=1.63-4.94) in a minimally adjusted model (LDL-C only) and 2.15 (CI==1.13-4.08) in a fully adjusted model (Ridker et al. 2012). No Kaplan-Meier analyses were published for JUPITER.

Based on positive clinical signal for risk prediction for CHD events in the analysis of the population upper quartile of Lp-PLA2 activity in the JUPITER placebo arm (a mixed gender study), a proposed binary cut point of 225 nmol/min/mL was selected and evaluated in available training studies with CHD outcomes data as described in this report.

Subjects with values below the cut point will be considered at reduced risk for CHD events (the low Lp-PLA2 group). Conversely subjects with values greater than or equal to the cut point will be considered at increased risk for CHD events (the high Lp-PLA2 group).

Training for the Cut Point

The relevant primary efficacy analysis for a clinical validation study of Lp-PLA₂ activity in the prediction of CHD events in subjects with no prior history of CVD is a Kaplan-Meier survival analysis between the low and high Lp-PLA₂ activity groups. The test for statistical difference between the Lp-PLA₂ sub-groups is a log-rank test at a 0.05 significance level (p<0.05) for demonstration of efficacy in risk prediction. The relevant secondary analysis is a Cox proportional hazards model to estimate the proportional hazards ratio between the high and low Lp-PLA₂ groups. High Lp-PLA₂ will be determined to be a significant predictor of events if (a) the point estimate of the FIR exceeds 1.0 and (b) the HR 95% CI does not overlap 1.0.

Two independent cardiovascular cohorts were analyzed as training studies to evaluate the cut point of 225 nmol/min/mL for PLAC Test for Lp-PLA₂ Activity for selection of the high and low groups in Kaplan Meier survival analysis and Cox proportional hazards model (utilizing JMP® statistical software version 10) for efficacy in the prediction of risk for coronary heart disease events in subjects with no prior CVD. Subjects from these two cohorts will not be used in subsequent clinical validation studies of the cut point.

Training Cohort 1

Training Cohort 1 was a prospective population-based cohort study designed to explore the effects of diet on cancer risk. All men and women (aged 46-73 years) in the study location were eligible for inclusion. The baseline blood draws were performed from 1991 to 1994 on 11,063 men and 17,035 women who enrolled in the study. The median follow-up time for the baseline samples was 10.6 years: CHD events were defined as non-fatal MI or death to ischemic heart disease.

Lp-PLA2 activity levels were measured in the cohort by a research use only manual microplate version of the Lp-PLA2 activity assay (CAM). A subpopulation of 141 samples was tested by both methods (CAM and PLAC Test for Lp-PLA2 Activity on the Beckman Coulter (Olympus) AU400) as a bridging study between the two formats in samples ranging from 83 to 308 nmol/min/mL (spanning the range of expected values). Sample values were re-value assigned to PLAC Activity values based on conversion by the regression parameters in this bridging study (PLAC Activity=1.281×CAM+17.9).

Kaplan Meier Survival Analysis.

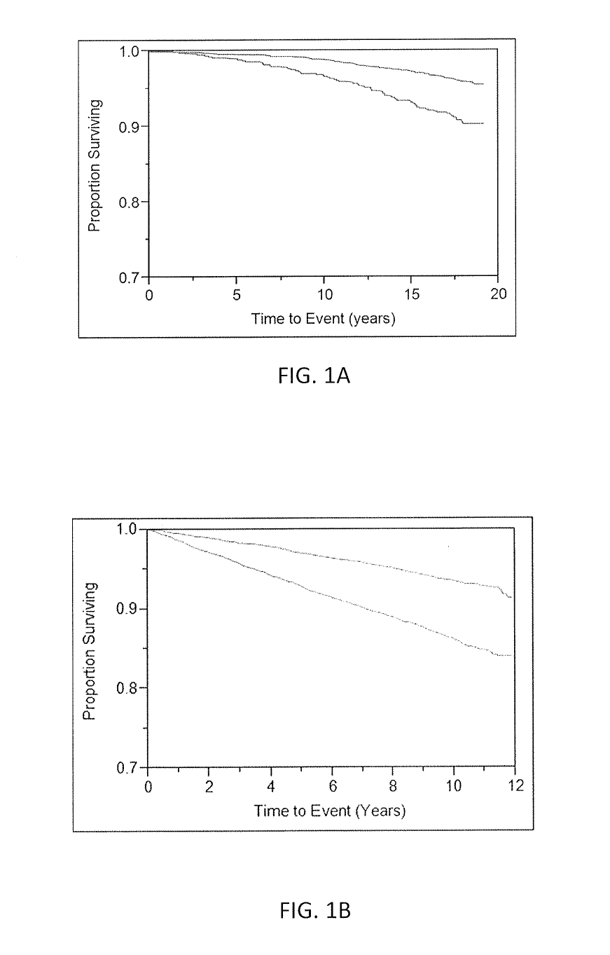

Utilizing a cut point of 225 nmol/min/mL for PLAC Test Lp-PLA2 Activity measurement as a binary categorical variable, the Training Cohort 1 baseline dataset was analyzed in plots of the Kaplan Meier survival estimates free from CHD events over time (FIG. 1A). A significant difference in the Kaplan-Meier survival estimator between the high and low Lp-PLA2 groups was demonstrated by the log-rank test (p<0.0001).

Cox Proportional Hazards Model.

In the univariate Cox proportional hazards model, a statistically significant hazard ratio was observed for the Training Cohort 1 baseline samples using the 225 nmol/min/mL cut point for PLAC Test Lp-PLA2 Activity: HR 2.3 (CI=1.71-3.14, p<0.0001).

Training Cohort 2

Training Cohort 2 was prospective longitudinal long-term biracial study (Caucasian, African American) of atherosclerotic cardiovascular disease incidence in subjects from 4 communities in the United States. Subjects were initially recruited between 1987 and 1989 in the age range of 45 to 64 years (n=15,792). Participants were followed up for incident CHD, defined by combinations of chest pain, ECG changes, cardiac enzyme levels, and surgical revascularization.

An investigational version of the PLAC Test for Lp-PLA2 Activity (not commercialized in the US) was used to measure Lp-PLA2 activity levels in plasma from Training Cohort 2, collected in 1994-1998 and preserved at ≦70° C. Samples were tested on the Beckman Coulter (Olympus) AU400. The study population analyzed had no prior CVD (n=9,549), the average age of subjects was 53-75 years (mean 62 years) and follow-up time was up to 12 years.

Kaplan Meier Survival Analysis.

Utilizing a cut point of 225 nmol/min/mL for PLAC Test Lp-PLA2 Activity measurement as a binary categorical variable, the Training Cohort 2 dataset was analyzed in a Kaplan Meier plot of survival estimates free of CHD events over time (FIG. 1B). A significant difference in survival between the high and low Lp-PLA2 groups was demonstrated by the log-rank test (p<0.0001).

Cox Proportional Hazards Model.

In the Cox proportional hazards model, a statistically significant hazard ratio was observed using the Training Cohort 2 baseline samples in the high Lp-PLA2 activity group defined by 225 nmol/min/mL as a binary cut point for the PLAC Test Lp-PLA2 Activity: HR 2.2 (CI=1.93-2.53, p<0.0001).

Discussion/Conclusions

An Lp-PLA2 activity value of 225 nmol/min/mL was proposed as the clinical cut point for risk prediction of CHD in subjects with no prior history of CVD based on positive clinical signal (hazard ratios >1.5 in various sub-groups) for risk prediction for CHD events in the placebo arm of the JUPITER (an independent, longitudinal, mixed gender cohort study). Subjects with values below the cut point were considered at reduced risk for CHD events. Conversely subjects with values greater than the cut point were considered at increased risk for CHD events.

The cut point of 225 nmol/min/mL was evaluated in two training studies measuring preserved baseline blood samples from the longitudinal cardiovascular cohorts, Training Cohort 1 and Training Cohort 2, by the PLAC Test for Lp-PLA2 Activity and analyzed by Kaplan Meier survival analysis with prospective CHD outcome data. Statistical differences were observed in both studies using the 225 nmol/min/mL cut point by Kaplan Meier survival analysis (p<0.0001), and both relative risk and Cox proportional hazard ratios were greater than 2.0 for both datasets indicating a doubling of the high Lp-PLA2 activity group's risk over the low group's risk.

Based on these analyses, the cut point of 225 nmol/min/mL is recommended for analysis of PLAC Test for Lp-PLA2 Activity results in its clinical validation study for risk prediction of coronary heart disease in patients with no prior history of cardiovascular disease.

### Example 2

### Clinical Validation Study

Overview

Clinical evidence of test efficacy in its intended use population was obtained in a case-cohort study utilizing baseline EDTA-plasma specimens from subjects enrolled in 2003-2007 and subsequently followed in the REGARDS study, a nationwide longitudinal observational study of cardiovascular diseases further described below.

Study Design

The Clinical Validation Study utilized a case-cohort study design selected from a broad, generalizable observational source cohort. This source cohort was chosen as representative of the range of patients commonly encountered by health care providers nationwide, in pragmatic, real-life clinical settings, with relatively few inclusion criteria.

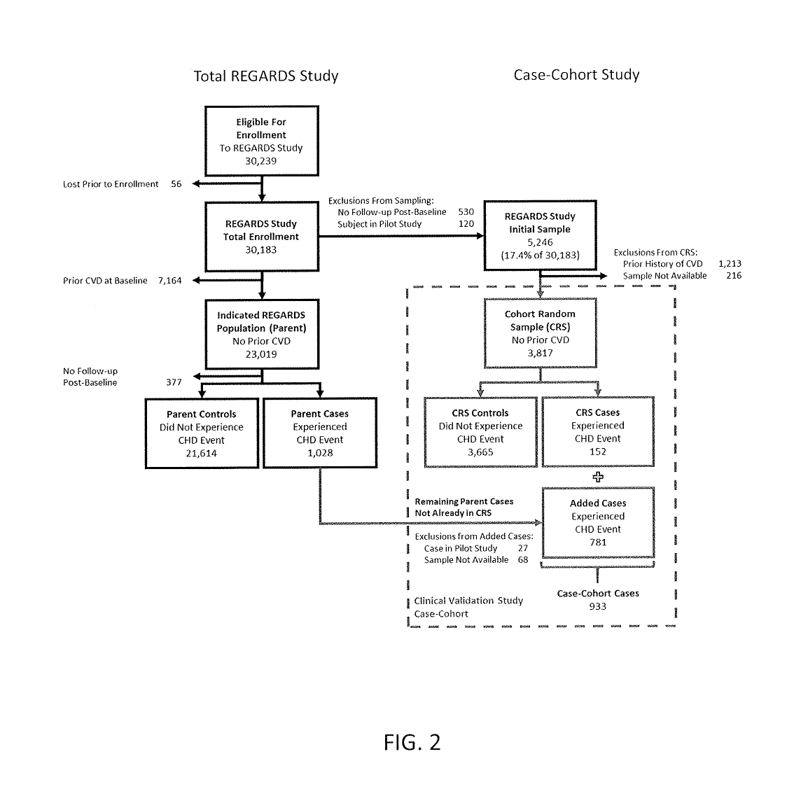

The case-cohort design is summarized in FIG. 2 and detailed in the following sections. In brief, a random sample was initially selected from the full REGARDS study, subsequently enrolling into the study only the subpopulation of selected participants having no prior history of cardiovascular events at baseline consistent with the intended use of the product (this selected sample is called the “Cohort Random Sample” or “CRS” in the following discussion). This CRS is then enriched by adding the remaining CHD cases (see Primary Clinical Endpoint (Total CHD Composite) section below) from the full REGARDS parent study after the same exclusion (the “Indicated REGARDS Population” or “Parent Population”). The combination of the CRS and remaining cases in a case-cohort study design comprises the complete validation subject set, referred to generically as the Clinical Validation Study or case-cohort in the following analyses and discussions.

The Clinical Validation Study's case-cohort design increases the efficiency and power of the study while allowing for the normal comparisons between the randomly-sampled CRS to the Indicated REGARDS Population as a whole (as well as between individual outcome groups such as cases and controls). With appropriate weighting of the sampled populations (see below), the underlying prevalence of the parent population can also be reflected, allowing the calculations of the same clinical study outcome statistics that would have been obtained using the entire cohort, were it available. These include calculations of event and survival rates (using Kaplan-Meier analyses) and hazard ratios (using Cox proportional hazards models), as performed in the Clinical Validation Study.

REGARDS Study Cohort

Study Summary.

The REasons for Geographic And Racial Differences in Stroke (REGARDS) study was used as the source study for the Clinical Validation Study. REGARDS is an active nationwide observational population study which continues to follow its participants longitudinally over time for the development of cardiovascular and other events. It is the largest such study ever undertaken in the United States by the National Institute of Health (NIH), with 30,183 participants enrolled.

REGARDS was designed to follow a broad, generalizable “all comers” population for the validation and elucidation of previously observed racial and geographical differences in the risk of stroke and coronary heart disease events. It is the first nationwide study sufficiently powered to relate cardiovascular event rate and case fatality, including pre-hospital deaths, to geographic and ethnic variations. The REGARDS study design, including its inclusion and exclusion criteria, has been published (Howard et al. 2005) and is summarized below. The REGARDS coronary heart disease outcomes were also recently peer-reviewed and published in the Journal of the American Medical Association (Safford et al. 2012).

Study enrollment began in 2003 and completed in 2007; participants have been followed continuously since their baseline interviews and in home examination. REGARDS adjudication of coronary heart disease events is now available through Dec. 31, 2010, representing a median follow-up of 5.3 years with the first enrolled subjects now approaching eight years since their enrollment.

Organization.

REGARDS is funded by the NIH National Institute of Neurological Disorders and Stroke (NINDS cooperative agreement U01 NS041588) and the National Heart, Lung, and Blood Institute (NHLBI R01 HL080477). The organization of REGARDS comprises an Operations Center and the Survey Research Unit (SRU) at the University of Alabama at Birmingham, a Central Laboratory at the University of Vermont, an Electrocardiogram (ECG) Reading Center at Wake Forest University, an in-home exam component provided by Examination Management Services, Inc. (EMSI), and a medical monitoring center at Alabama Neurological Institute, Inc. An Executive Committee comprising the principal investigator of each study center and a National Institute of Neurological Disorders and Stroke representative assists the principal investigator at the University of Alabama at Birmingham in the scientific leadership of the study. Study methods were reviewed and approved by all involved institutional review boards, as well as an external observational study monitoring board appointed by the funding agency.

The study conforms to the principles of the Declaration of Helsinki and was approved of by the local ethical committees. All participants provided written informed consent. REGARDS is a registered longitudinal observational trial.

Enrollment Criteria.

Criteria for inclusion in the cohort were deliberately broad and generalizable to the US population. The REGARDS study was recruited from a commercially available nationwide population sample purchased through Genesys Inc., stratified to reflect the specific strata described below. Criteria for inclusion in the sample include having a name, telephone number and address in the Genesys database. Upon reaching a household resident, the household was enumerated and one resident aged ≧45 was randomly selected and screened for eligibility.

The original REGARDS recruitment goal of 30,000 participants included 50% from the 11 “stroke belt” and “stroke buckle” states (AL, AR, GA, IN, KY, LA, MS, NC, SC, TN and VA) and 50% from the remaining 37 contiguous states of the continental U.S. Within each region, a goal was that approximately one half were to be white and one half to be African-American, and within each region-race stratum, approximately one half male and one half female.

Criteria for exclusion from the cohort were deliberately limited so as to access the broadest US population for the full longitudinal follow-up of the study. The exclusion criteria included race other than African-American or white, active treatment for cancer, medical conditions that would prevent long-term participation (i.e. terminal illnesses at initial interview), cognitive impairment judged by the telephone interviewer, residence in or inclusion on a waiting list for a nursing home, or inability to communicate in English. Potential participants who responded ‘don't know’ to questions about medical conditions were nonetheless considered eligible for the study.

Baseline Assessment. Following informed consent by participants, the medical history, including risk factor evaluation, was collected by computer-assisted telephone interviewing (CATI). Following the telephone interview, the participant's contact information was transmitted to EMSI for scheduling of an in-home visit. EMSI technicians trained on methods for the REGARDS protocol completed the in-home visits and shipped samples to the central laboratory. Physical measurements, a resting ECG, medication inventory, phlebotomy and urine collection were performed using standardized methods.

Study Design and Power.

The target 30,000 subject sample size of REGARDS was calculated to provide a sufficient number of cardiovascular events to detect associations with risk factors with relatively small differences in risk (i.e. small hazard ratios). The initial design was powered to study the stroke component endpoint individually amongst major cardiovascular endpoints. However, all major cardiovascular event endpoints are followed (including CHD, MI, etc.) and use the same REGARDS parent cohort, following all REGARDS participants for the entire duration of the study.

A total of 30,183 participants ≧45 years old were enrolled in the study with baseline blood specimens drawn between February 2003 and October 2007 (30,239 accepted with 56 lost prior to enrollment visit). This makes the resulting REGARDS cohort the largest single longitudinal cardiovascular public health study ever undertaken in the United States by the NIH, far exceeding the original Framingham (n=5,209), Framingham Offspring (n=5,124), Cardiovascular Health Study (CHD; n=5,888), Atherosclerosis Risk in Communities (ARIC; n=15,792), Multi-Ethnic Study of Atherosclerosis (MESA; n=6,800), and Coronary Artery Risk Development in Young Adults (CARDIA; n=5,115) studies.

Unlike these prior studies, which recruited and/or followed up patients enrolled at a limited number of healthcare centers, REGARDS is uniquely nationwide in scope. Subjects were recruited in their own homes, coming from all 48 contiguous states.

Participant Follow-Up.

The REGARDS participants are followed prospectively for a variety of health outcomes. REGARDS staff conducts active surveillance of cohort members, contacting all participants by telephone semi-annually to collect data on both suspected CVD events that require hospitalization as well as any adverse cardiovascular symptoms using a consistent computer aided telephone interview (CATI) questionnaire, subsequently collecting medical records and adjudicating all potential events identified. If a participant is hospitalized or sees a physician for such symptoms, contact information for the hospital and/or physician is obtained from the participant, and pertinent in- and outpatient medical records are sought.

Similarly, REGARDS staff identifies all deaths amongst those lost to follow-up, through participant proxies/next-of-kin as well as through various governmental social security indices and death records, and collects all available death and medical records for potential event adjudication using established epidemiological and clinical study practices. If a death is reported, the death certificate and associated hospital or physician records are collected, including medical records for the 28-day period preceding death. If death occurred within a month following a procedure, information on that procedure is collected. If medical records are unavailable or judged insufficient by the adjudication committee, a physician questionnaire for decedents or an informant interview questionnaire is completed.

Assessment of Clinical Endpoints.

Cardiovascular disease adjudication in REGARDS includes both CHD events and cerebrovascular events such as stroke. REGARDS retrieves medical records for all hospitalizations among REGARDS participants and collects data on all deaths. These records are then adjudicated, with cardiac procedures and medications collected and findings added to the extensive data already collected by REGARDS ongoing participant surveillance operations.

Adjudication of REGARDS events is overseen by two independent Adjudication Committees: one for stroke, and the other for heart and death outcomes. Coronary heart disease endpoints followed in the REGARDS study include myocardial infarction, coronary insufficiency requiring revascularization procedures, and death from CHD (ICD-10-CM diagnosis codes 120-125). Adjudicators for coronary heart disease (CHD) outcomes are physicians familiar with general internal medicine principles and in particular with the diagnostic work-up of cardiovascular disease based on established guidelines (Luepker et al. 2003). Individual adjudicators are masked to participant race.

The expert adjudication of CHD outcomes for the REGARDS study thus reflects medical records from over 1,500 hospitals across the US, ensuring a standard of care representative of that seen nationally in current clinical practice. In REGARDS, fully adjudicated outcomes for all acute CHD events, including myocardial infarction, coronary revascularization procedures, and CHD-related death (the Primary Clinical Endpoint, see below), were recently updated through Dec. 31, 2010.

Biochemical Sampling Information.

Venous blood samples were obtained at the study enrollment in-home examination visits, processed, and stored at ≦−70° C. until time of the PLAC Activity measurement, with no more than two freeze-thaw cycles prior to such testing.

Indicated REGARDS Population

Consistent with the proposed intended use, the Clinical Validation Study excluded subjects that had a prior history of cardiovascular disease at baseline (CVD events; including both CHD and stroke events). The study analyzes specimens taken only from those participants in REGARDS without a prior history of CVD at baseline (the parent “Indicated REGARDS Population”) with available banked samples. No further clinical inclusion or exclusion criteria were imposed beyond those described previously for the original cohort study.

The total REGARDS cohort is comprised of 30,183 subjects; the Indicated REGARDS Population utilized in Clinical Validation Study comprises solely the 23,019 subjects with no prior history of CVD events at baseline.

The total counts of the Indicated REGARDS Population cases for the CHD endpoints analyzed in this protocol were based on censor/study lock and adjudication to Dec. 31, 2010. Up to this date, 1,028 subjects in the Indicated REGARDS Population had experienced CHD events.

Case-Cohort Study Design

Overall Design.

The Clinical Validation Study uses a case-cohort study design (Prentice 1986). A simple random sample was taken from the total REGARDS cohort (exclusions from eligibility for sampling eliminated subjects with no post-baseline follow-up (530 in total REGARDS; 377 in the Indicated REGARDS Population) or that had participated in an initial pilot study (120 in total REGARDS; 60 in the Indicated REGARDS Population, 33 controls and 27 cases), and subjects within the Indicated REGARDS Population were selected (the Cohort Random Sample or CRS). This sample was then enriched with all remaining subjects within the Indicated REGARDS Population who experienced a Primary Clinical Endpoint event (see below) that had not already been previously selected within the CRS. The combination of the CRS and remaining cases comprises the complete subject set, referred to generally as the Clinical Validation Study in the following discussions.

Case-Cohort Study Subject Weights.

Due to the above case enrichment, cases are over-represented in the case-cohort compared to in the full cohort. As a result, subject weight adjustments are required in a case-cohort design for outcomes analyses and estimates of the underlying incidence rates in the full cohort.

The case-cohort weighting method of Barlow (Barlow 1994) was used for the case-cohort study following review of available methods (Barlow et al. 1999). The Barlow case-cohort weighting method is an inverse probability weighting (IPW) methodology, and builds upon more generalized work on sampling weights in survey and clinical study design (Horvitz & Thompson 1952; Korn & Graubard 2011; Gelman & Carlin 2000).

The Barlow method weights each CRS control by the inverse of the CRS sampling fraction within the full cohort. The method allows the CRS controls to be “up-weighted” back to the full cohort population from which they were chosen. As a simple random sampling method was used in the Clinical Validation Study, only a single weight was needed for the entire population of control subjects utilized in the case-cohort study. An IPW weight of 5.75 was used for controls, which is the inverse of the 17.4% initial overall sampling probability from the full REGARDS study.

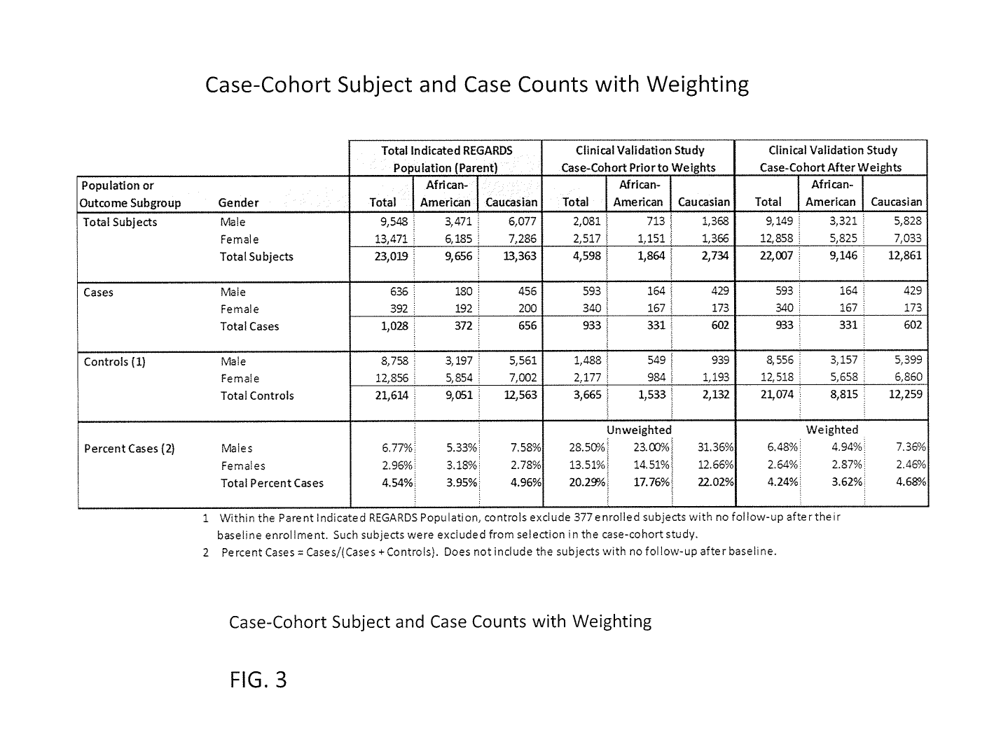

Since all available cases are utilized, the subject weight for cases remains at the same base weight as in a full cohort analysis. An IPW weight of 1.00 is employed, the inverse probability of the 100% sampling fraction for cases. This is applied per Barlow to both the cases from within the CRS and also to the remaining Indicated REGARDS Population cases added in the full case-cohort study (see FIG. 3). (Note: In this conservative “intent to test” approach to weighting using the Barlow methodology, neither cases nor controls are up-weighted further to offset subjects lost due to missing samples, without post-baseline follow-up, or that had been previously used in the pilot study analyses; such losses instead reduce the power of the case-cohort study. Only a single simple probability rate is used, based on the highest level of the sample (REGARDS enrollment prior to exclusions or other losses), without further stratification.)

These case-cohort IPW weights are used in all longitudinal analyses to generate absolute risk estimates from Kaplan-Meier analyses and hazard ratios from Cox proportional hazards regression estimates. Descriptive cross-sectional comparisons involving the CRS sample alone, or of cases or controls taken as individual subgroups, do not require IPW weights as each individual subject in such subgroups share the same original sampling probability and thus IPW weight.

The resulting Clinical Validation Study case-cohort consisted of a Cohort Random Sample of 3,817 (3,665 controls, 152 cases) enriched with a remaining 781 cases from the Indicated REGARDS Population. Taken together, the case-cohort sums to a total of 4,598 subjects, across the 3,665 controls (the “CRS Controls”) and 933 total cases (the “Case-Cohort Cases”). These terms (CRS, CRS Controls, Case-Cohort Cases) are used throughout the following discussions and analyses to more precisely distinguish the various subpopulations and outcome subgroups used in the Clinical Validation Study.

Clinical Endpoints

Within the Clinical Validation Study, subjects were followed longitudinally after baseline examination for subsequent CHD events of interest, allowing analysis of the following clinical endpoints:

Primary Clinical Endpoint (Total CHD Composite).

The primary clinical outcome variable and event of interest used was a composite endpoint of total CHD events. A “Primary Clinical Endpoint” event (hereafter simply referred to as an “event” or a “CHD event”) was defined as (a) acute myocardial infarction, (b) coronary revascularization or (c) CHD-related death.

Secondary Clinical Endpoints (CHD Components).

Other secondary clinical outcome variables and events of interest comprised the individual component outcomes (“Secondary Clinical Endpoints”) contained within the composite Primary Clinical Endpoint, which were used in supportive analyses; specifically, (a) acute myocardial infarction, (b) coronary revascularization and (c) CHD-related death analyzed individually.

Any Secondary Clinical Endpoints and their related events are specifically identified as such below, including in figures and tables.

Event and Case Counts by Clinical Endpoint

In Table 2 below, the number of total and tested cases is given for each event of interest, providing counts for both the total Indicated REGARDS Population and the tested REGARDS participants in the Clinical Validation Study. Of the 1,028 total CHD cases in the parent REGARDS Indicated Population, samples were available for 933 (90.8% of the total cases).

TABLE 2

| | Parent Cases | Case-Cohort | Tested/ |
|---|---|---|---|
| | Indicated | Cases Clinical | Total |
| Event of Interest | REGARDS | Validation | (% Ascer- |
| (Abbreviation Used) | Population | Study | tainment) |
| Coronary Heart Disease | 1,028 | 933 | 90.8% |
| Composite Endpoint (CHD; | | | |
| Primary Clinical Endpoint) | | | |
| Myocardial Infarction (MI) | 523 | 467 | 89.3% |
| Coronary Revascularization | 634 | 591 | 93.2% |
| (Revasc) | | | |
| CHD-Related Death | 262 | 221 | 84.4% |
| (CHD Death) | | | |

Each component Secondary Clinical Endpoint in the table is presented adjudicated independently of the other components. Consistent with normal practice, the first to occur event for a given clinical endpoint is utilized here and in any subsequent statistical analyses. Time to event is calculated as the time from a REGARDS participant's baseline in-home interview date (tested sample draw date) to the occurrence of that first (incident) event of interest experienced by that participant. Subsequent (non-incident) events (of any type) experienced by the subject are thus right censored.

As a result, participants experiencing events are only counted once as a case within any given clinical endpoint, regardless of the number of such events such subject may have actually experienced. Counts of events represent individual unique participants, and are thus the presented as to cases.

As many subjects experienced more than one of the events of interest across different components during the study, the sum of the cases across all component CHD endpoints will also be greater than the number of cases shown in the total composite of CHD events. For example, a participant experiencing one (or more) coronary revascularizations before dying a CHD-related death would be counted as one case in each of the two component Secondary Clinical Endpoint analyses, the first coronary revascularization and the death, respectively, but only the first coronary revascularization event would be counted as a case in the CHD composite Primary Clinical Endpoint.

Statistical Methods

Statistical methods used were pre-specified the Statistical Analysis Plan. Statistical models and methods were also evaluated for their appropriateness in each analysis in pre-specifying such plans, with such assumptions confirmed in the actual analyses (e.g. Cox proportional hazards models assessed for violation of the proportional hazards assumption, etc.). No deviations or changes from the pre-specified SAP were required in the conduct of the analyses.

Study Analysis Sets (FAS, WAS, BAS).

In order to explore results by race group, the Clinical Validation Study protocol employed three different analysis sets, which are labelled below as follows:

Full Analysis Set (“FAS”) consisting of the full case-cohort as described above.

White Analysis Set (“WAS”) consisting solely of the Caucasian subjects.

Black Analysis Set (“BAS”) consisting solely of the African-American subjects.

In addition to the full study analysis set, the two racial subpopulations formed two additional non-overlapping analysis sets. The results in each race subgroup analysis set are also presented for the primary and secondary efficacy analyses, and in the supportive subgroup analyses by race.

PLAC Activity Values and Analysis Cut Point.

Cut Point Analyses.

Following the SAP, for the primary and secondary clinical efficacy analyses of the Clinical Validation Study, PLAC Activity values were analyzed as a binary categorical variable with individual PLAC Activity results dichotomized using the pre-specified analysis cut point of 225 nmol/min/mL (Cut point Analyses). Subjects with values below the Analysis Cut point were assigned to the “low PLAC Activity group.” Conversely, subjects with values greater than or equal to the Analysis Cut point were assigned to the “high PLAC Activity group.”

Continuous Analyses.

Continuous PLAC Activity values are also used directly (un-transformed) as pre-specified for the various descriptive analyses of the Clinical Validation Study.

Subsequent to the Cut point Analyses, the underlying continuous PLAC Activity results were also pre-specified as available for use in the other supportive clinical efficacy analyses and to the extent needed to achieve a well-powered analysis (see Sample Size and Power Assessment below).

The results of the Cut point Analyses and race interaction tests were such that this last procedure was not required for power (e.g., the efficacy analyses of the Clinical Validation Study were sufficiently powered to achieve a statistically significant result), but such results are presented as pre-specified parallel analyses (see PLAC Activity Continuous Analyses section below). In such continuous analysis, the PLAC Activity results were log transformed (log₂) in accordance with the SAP.

Other Dataset Variables.

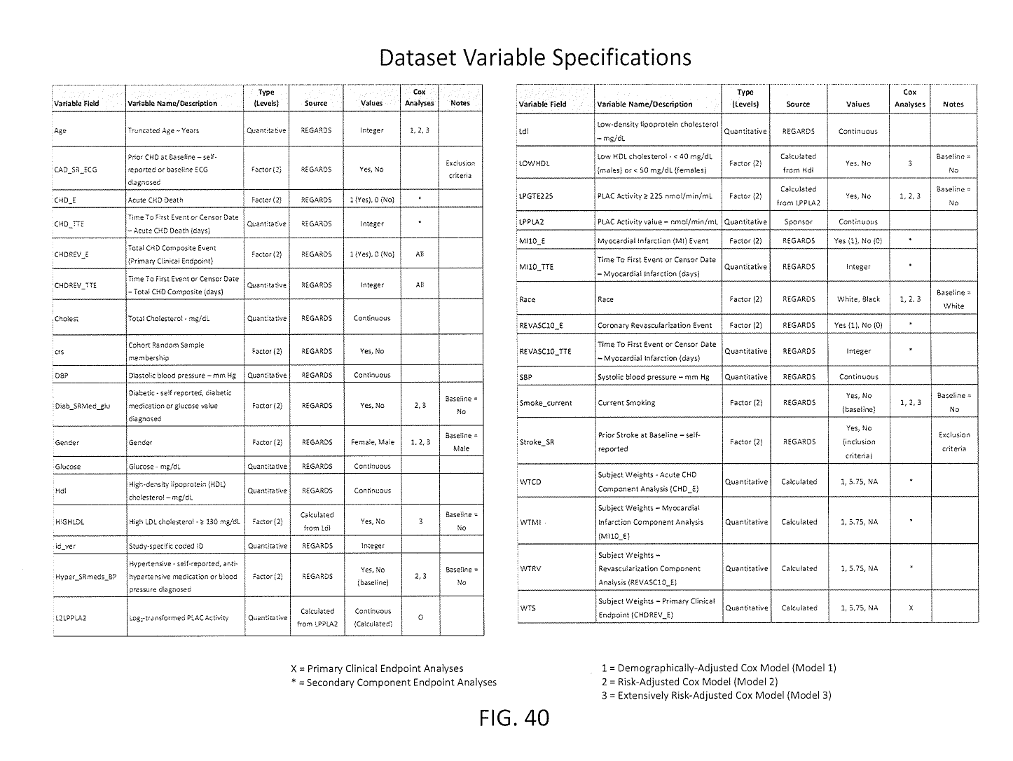

Each variable was pre-specified in the SAP as shown in FIG. 40 (Dataset Variable Specifications), which includes transformations and dichotomization of the covariates used in the risk-adjusted multivariate Cox proportional hazards analyses. The dataset field names provided are those used in the provided data files for all the studies of the ClinicalProgram.

Other dataset and Case-Cohort Methods information for FIG. 40: Censor date is the last event adjudication cutoff, Dec. 31, 2010. Following R (www.r-project.org) conventions, all missing subject data is shown as “NA”. The full Indicated REGARDS Population baseline annotations (n=23,019) is provided for comparisons to parent cohort only. Only subjects with tested PLAC Activity values are members of the Clinical Validation Study (n=4,598). Subjects who were not part of the Clinical Validation Study case-cohort have NA for their PLAC Activity values (n=18,421). CRS membership field (crs=Yes) is prior to eliminations for sample availability and pilot study exclusion, so includes subjects who could not be tested for PLAC Activity (total n=4,034); These subjects are not part of the Clinical Validation Study Full Analysis Set, and have NA for their PLAC Activity Values (not in study n=217). Primary Clinical Endpoint cases field (CHDREV_E=1, total n=1,028) is also prior to such adjustments (not in study n=95). Within the tested subjects only, the total case-cohort subjects (n=4,598) are the set union of the total Cohort Random Sample members (crs=Yes, tested n=3,817) and the Case-Cohort Cases using the Primary Clinical Endpoint (CHDREV_E=1, tested n=933). Within the tested subjects only, the set intersection of CRS members and Case-Cohort Cases are the CRS Cases only (n=152). In weighted component endpoint analyses, NA is also used in the subject weight fields in order to indicate Clinical Validation Study subjects outside of the CRS (crs=No) who experienced the Primary Clinical Endpoint, but not the component event of interest. These subjects are excluded from the smaller case-cohort used in such component analyses.

The following pre-specified covariates were used in the multivariate Cox model analyses of the PLAC Activity Test in the Clinical Validation Study: age, gender, race, diabetes, hypertension, current smoking, LDL and HDL (which were dichotomized). These covariates are the same as those used in the Framingham Risk Score, with the two cholesterol covariates (LDL and HDL) dichotomized according to their ATP III category recommendations and current risk assessment guidelines (Wilson et al. 1998; ATP III. NHLBI 2002; Goff et al. 2013).

Case-Cohort Weighting.

Following the SAP, case-cohort weighted analyses are given consistently as the main results for all efficacy analyses (such as Kaplan-Meier analyses and Cox models), which includes all point estimates and confidence intervals of absolute event rates and hazard ratios. These statistics are consistently weighted to reflect the underlying true prevalence of the full Indicated REGARDS Population using subject sample weights as described in the Case-Cohort Weighting section above.

As an additional analysis to the SAP, the same statistics are also provided in parallel without the use of sample weights (unweighted) for the main Cox model analyses. Where weighted and unweighted results are presented together in an analysis, each is separately labelled. In the absence of such labelling, all efficacy analyses should be assumed to be after the application of case-cohort weights.

Weighted confidence intervals are similarly provided as the main results for all efficacy analyses. However, p-value statistics provided for log-rank tests and hazard ratios are conservatively calculated without the use of sample weights in order to rely solely on the tested sample set (i.e. without distributional assumptions) and also to maximize the independent reproducibility of the analyses (versus using resampling or other methods more amenable to sample weights).

The descriptive analyses of the Clinical Validation Plan utilize subgroups pre-specified in the SAP so as to not require sample weighting, and so are appropriately presented unweighted. For example, the Cohort Random Sample is compared alone (without the enrichment of the additional cases) directly to the parent Indicated REGARDS Population from which it is sampled, and the full case-cohort cases are compared directly to all cases in the parent Indicated REGARDS Population from which they are sampled.

Missing Data.

Although the SAP protocol allowed imputation of non-PLAC Activity values under certain conditions, this was not required. Missing values were not imputed for PLAC Activity values nor for any other study covariates in any study analyses.

Where one or more covariates were required in the various supportive Cox analyses shown, only the subjects with complete covariate data are employed, and the reduced count of total subjects and events is given in parallel with each such result below. Subjects missing any needed follow-up or covariate data were eliminated from such analyses. In the extensively risk-adjusted Cox models, which included the full set of covariates, this approach reduced the full case-cohort study set (FAS) to 4,439 subjects and 898 total CHD events (from the full 4,598 subjects and 933 CHD events in the full case-cohort study without any exclusions).

Sample Size and Power Assessment.

As these are baseline samples from an existing enrolled study, the parent Indicated REGARDS Population sample size was not pre-determined based on the Primary Clinical Endpoint of this Clinical Validation Study. However, based on the actual sample size and CHD events that had occurred to the latest REGARDS adjudication date of Dec. 31, 2010, a power assessment was performed as part of the SAP preparation, and is summarized here for the validation study analyses by FAS, WAS, and BAS, and for subgroups and component endpoints within such analysis sets (see the SAP for further detail).

The various analyses of the validation study were considered statistically well-powered if they were estimated to have at least 90 percent power (1−β≧0.90) to detect a pre-specified relative risk or proportional hazard ratio for total events at a two-sided significance level of 5 percent (α=0.05). In the Cox proportional hazards analyses, covariate correlation with PLAC Activity was assessed at a variance inflation factor (VIF) ranging from 1.0 (R²=0.0) in univariate analyses to 2.0 (R²=0.5) in extensively risk-adjusted analyses using continuous variables (Hsieh & Lavori 2000; Schoenfeld 1983). These estimates were based on assumptions from prior independent studies and publications using Lp-PLA₂ activity (external to the Clinical Program).

In the pre-study power assessment, the Clinical Validation Study was estimated to be well-powered for the primary and secondary efficacy analyses, and specifically to detect an assumed univariate Cox proportional hazard ratio of 1.3 or greater for the Cut point Analyses using the Primary Clinical Endpoint in the FAS, WAS, or BAS.

More than the 933 CHD cases available for analysis in the FAS (602 in WAS and 331 in BAS) were required for the other supportive analyses to also be considered well-powered. In such supportive analyses, it was estimated that 1,212 cases would be required to be well-powered to detect the same 1.3 hazard ratio risk in a multivariate Cox model extensively risk-adjusted for all pre-specified covariates in a Cut point Analysis, which was reduced to 633 cases if continuous PLAC Activity values were employed as a Continuous Analysis. For this reason, none of the subgroup and component endpoint supportive analyses were originally estimated to be well-powered.

As will be shown below in the study results, the actual hazard ratios observed in the Clinical Validation Program were higher than those assumed in the SAP power assumptions. The lack of a PLAC Activity group by race interaction also allowed supportive analyses to be performed in the full analysis set with both races combined together, allowing each supportive analysis to include more CHD events. This improved the power of the actual Clinical Validation Program significantly.

Sample Size and Power Assessment

As these are baseline samples from an existing enrolled study, the parent Indicated REGARDS Population sample size was not pre-determined based on the Primary Clinical Endpoint of this Clinical Validation Study. However, based on the actual sample size and CHD events that had occurred to the latest REGARDS adjudication date of Dec. 31, 2010, a power assessment was performed as part of the SAP preparation, and is summarized here for the validation study analyses by FAS, WAS, and BAS, and for subgroups and component endpoints within such analysis sets (see the SAP for further detail).

The various analyses of the validation study were considered statistically well-powered if they were estimated to have at least 90 percent power (1−β≧0.90) to detect a pre-specified relative risk or proportional hazard ratio for total events at a two-sided significance level of 5 percent (α=0.05). In the Cox proportional hazards analyses, covariate correlation with PLAC Activity was assessed at a variance inflation factor (VIF) ranging from 1.0 (R²=0.0) in univariate analyses to 2.0 (R²=0.5) in extensively risk-adjusted analyses using continuous variables (Hsieh & Lavori 2000; Schoenfeld 1983). These estimates were based on assumptions from prior independent studies and publications using Lp-PLA₂ activity (external to the Clinical Program).

In the pre-study power assessment, the Clinical Validation Study was estimated to be well-powered for the primary and secondary efficacy analyses, and specifically to detect an assumed univariate Cox proportional hazard ratio of 1.3 or greater for the Cut point Analyses using the Primary Clinical Endpoint in the FAS, WAS, or BAS.

More than the 933 CHD cases available for analysis in the FAS (602 in WAS and 331 in BAS) were required for the other supportive analyses to also be considered well-powered. In such supportive analyses, it was estimated that 1,212 cases would be required to be well-powered to detect the same 1.3 hazard ratio risk in a multivariate Cox model extensively risk-adjusted for all pre-specified covariates in a Cut point Analysis, which was reduced to 633 cases if continuous PLAC Activity values were employed as a Continuous Analysis. For this reason, none of the subgroup and component endpoint supportive analyses were originally estimated to be well-powered.

As will be shown below in the study results, the actual hazard ratios observed in the Clinical Validation Program were higher than those assumed in the SAP power assumptions. The lack of a PLAC Activity group by race interaction also allowed supportive analyses to be performed in the full analysis set with both races combined together, allowing each supportive analysis to include more CHD events. This improved the power of the actual Clinical Validation Program significantly.

Baseline Descriptive Statistics.

Clinical Characteristics.

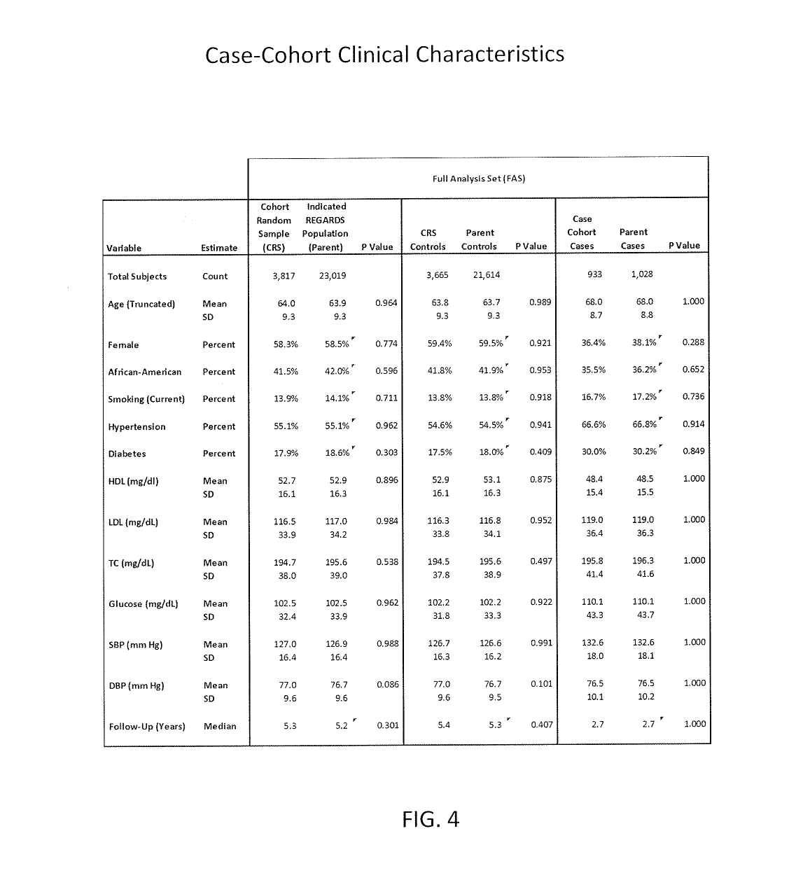

FIG. 4 summarizes the baseline clinical characteristics of the populations enrolled in the Clinical Validation Study, together with a comparison of the same characteristics in the relevant subgroups of the full Indicated REGARDS Population as the “Parent” cohort.

The clinical characteristics shown include all baseline variables used within the Clinical Validation Study. The CRS is compared to the Indicated REGARDS Population, being an equal probability sample of it, and the total Case-Cohort Cases (including both those within the CRS and also those added cases from outside the CRS) are compared to the total cases within the Indicated REGARDS Population, being the parent population from which such cases are taken. The test used above was a Kolmogrov-Smirnov test comparing the clinical characteristic distributions between each pair of populations, reporting p-values above; no statistically significant differences in any clinical characteristic were found in the sample.

The comparison indicates that the Clinical Validation Study closely matches the clinical characteristics of the parent Indicated REGARDS Population from which it was sourced, and comprises a representative sample of the full cohort, cases and controls.

The final racial and gender baseline enrollment demographics of the Clinical Validation Study population, as measured by the CRS as the unbiased sample of the Indicated REGARDS Population, is 41.5% African-American and 58.5% Caucasian, and 41.7% male and 58.3% female. The mean baseline age of subjects in the CRS is 64 years (median 63; range 45 to 92 years). The median study follow-up of the CRS members is 5.3 years; this date is calculated after right censoring at the earliest of last study follow-up, first CHD event (if any), or the Dec. 31, 2010 latest case adjudication date.

Subjects experiencing the Primary Clinical Endpoint of total CHD events (the “Case-Cohort Cases”) total 933 in the case-cohort population, with CHD cases representing 4.2% of subjects after application of case-cohort weights (933÷(3,665*5.75+933)=cumulative weighted case-cohort estimate). This is comparable to the 4.5% actual cases to subjects in the parent cohort (1,028÷(21,614+1,028)=actual cumulative CHD event rate), with the difference due to sample availability for the case-cohort (see FIG. 3 for further data by race and gender strata).

The median time to CHD event rate in the Case-Cohort Cases was 2.7 years through Dec. 31, 2010, indicating that a roughly linear accrual of CHD events has occurred over the follow-up period to the last available case adjudication date.

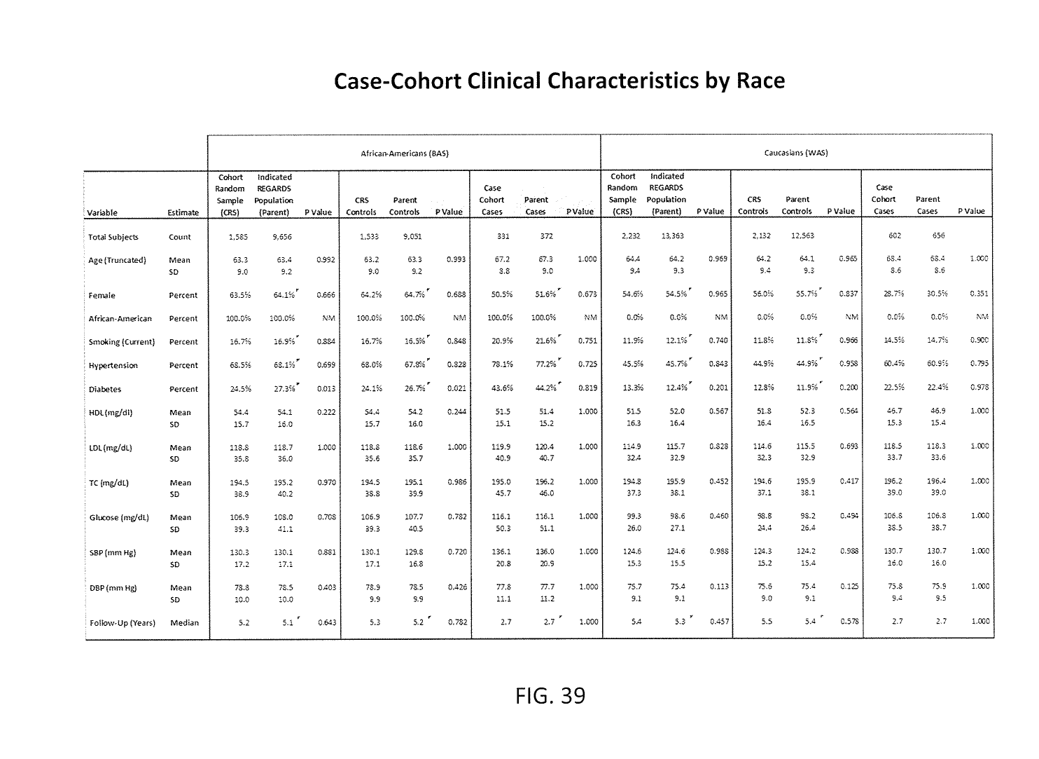

These and all other CRS clinical characteristics described above are comparable to the parent Indicated REGARDS Population. The same table of clinical characteristics is presented within each individual race analysis set (BAS and WAS) in FIG. 39

PLAC Activity Population Values (Reference Range.

PLAC Activity Population Values (Reference Range).

Because of its large size, nationwide recruitment, and well-documented intake inclusion and exclusion criteria, REGARDS also represents an ideal reference range study of PLAC Activity in the general intended use population. The Cohort Random Sample in the Full Analysis Set (FAS) is an unbiased simple random sample of the parent Indicated REGARDS Population, and thus represents the best direct estimate of PLAC Activity population values.

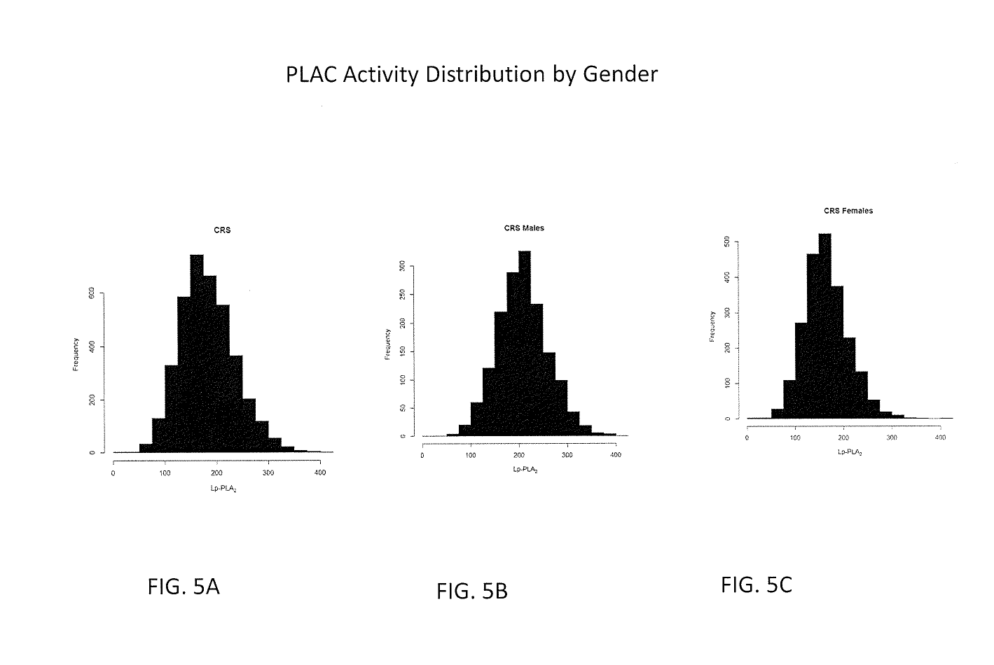

FIGS. 5A, 5B, and 5C present the population value distributions measured for PLAC Activity within the CRS.

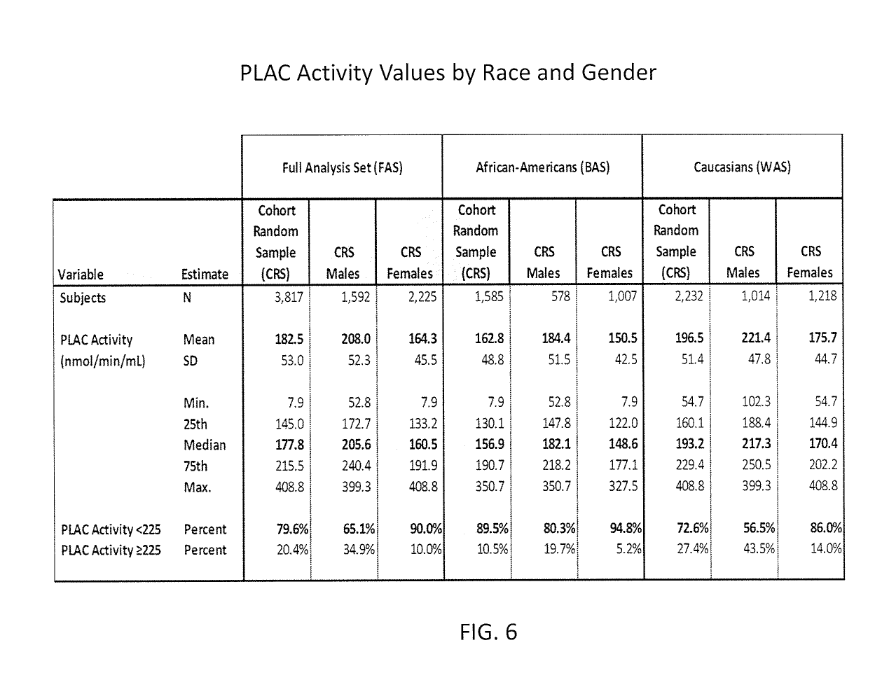

Following accepted protocols from CLSI (CLSI 2010), reference intervals were calculated using the CRS for full study (FAS) and within each race (BAS, WAS), for each gender individually and combined, using simple non-parametric statistics, without elimination of outliers (given the large count of subjects employed). FIG. 6 tabulates the resulting reference range value distributions (mean, standard deviation, inter-quartile range (IQR), median, min and max) for PLAC Activity for the full case-cohort (FAS), as well as by within each individual race analysis set (BAS and WAS).

Because the Analysis Cut point falls within the 95% reference interval, the interquartile range is presented per the CLSI manufacturer presentation guidance. The non-parametric percentile position represented by the Analysis Cut point is also shown, giving the percentages of each given population below (the low PLAC Activity group) and at or above (the high PLAC Activity group) the cut point.

PLAC Activity in the full CRS ranged from a minimum of 7.9 to a maximum of 408.8 nmol/min/mL, with a median value of 177.8 nmol/min/mL (IQR 145.0-215.5 nmol/min/mL). The PLAC Activity Analysis Cut point of 225 nmol/min/mL represents the 79.6^{th} CRS population percentile (non-parametric) within the CRS sample of the overall parent Indicated REGARDS Population.

As shown above, the African-American race has lower PLAC Activity values than the Caucasian race (median value of 156.9 vs. 193.2 nmol/min/mL in BAS vs. WAS). The Analysis Cut point represents the 89.5^{th} percentile in the BAS (African American CRS population) and the 72.6^{th} CRS population percentile in the WAS (Caucasian CRS population).

Furthermore, females have lower PLAC Activity values than males (median value of 160.5 vs. 205.6 nmol/min/mL in females vs. males). For females, the Analysis Cut point represents the 90.0^{th} CRS population percentile in the FAS, the 94.8^{th} percentile in the BAS, and the 86.0^{th} percentile in the WAS. For males, the Analysis Cut point represents the 65.1^{th} CRS percentile in the FAS, the 80.3^{th} percentile in the BAS, and the 56.5^{th} percentile in the WAS.

The direction of these PLAC Activity differences by each race and gender were not unexpected: they are broadly consistent with the differences in CHD event rates that were subsequently experienced in the Indicated REGARDS Population by such subgroups. African-Americans and females have lower CHD event rates than Caucasians and Males in the parent Indicated REGARDS Population, as well as in several other public health studies (see FIG. 3). The relationship of PLAC Activity values and subsequent CHD event rates is the subject of the Clinical Validation Study and is extensively analyzed below.

Nonetheless, the PLAC Activity value distributions of each of the subgroups showed substantial overlap across the races. For example, the interquartile range of White males (the highest PLAC Activity subgroup; IQR 188.4-250.5 nmol/min/mL) encompassed 42% of the interquartile range of Black males (IQR 147.8-218.2 nmol/min/mL). Likewise, the interquartile range of Black females (the lowest PLAC Activity subgroup; IQR 122.0-177.1 nmol/min/mL) encompassed 44% of the interquartile range of White females (IQR 144.9-202.2 nmol/min/mL).

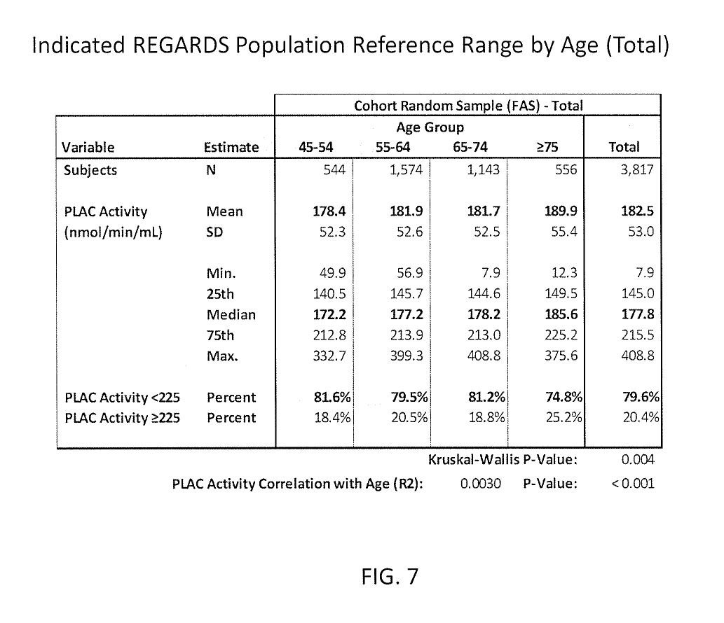

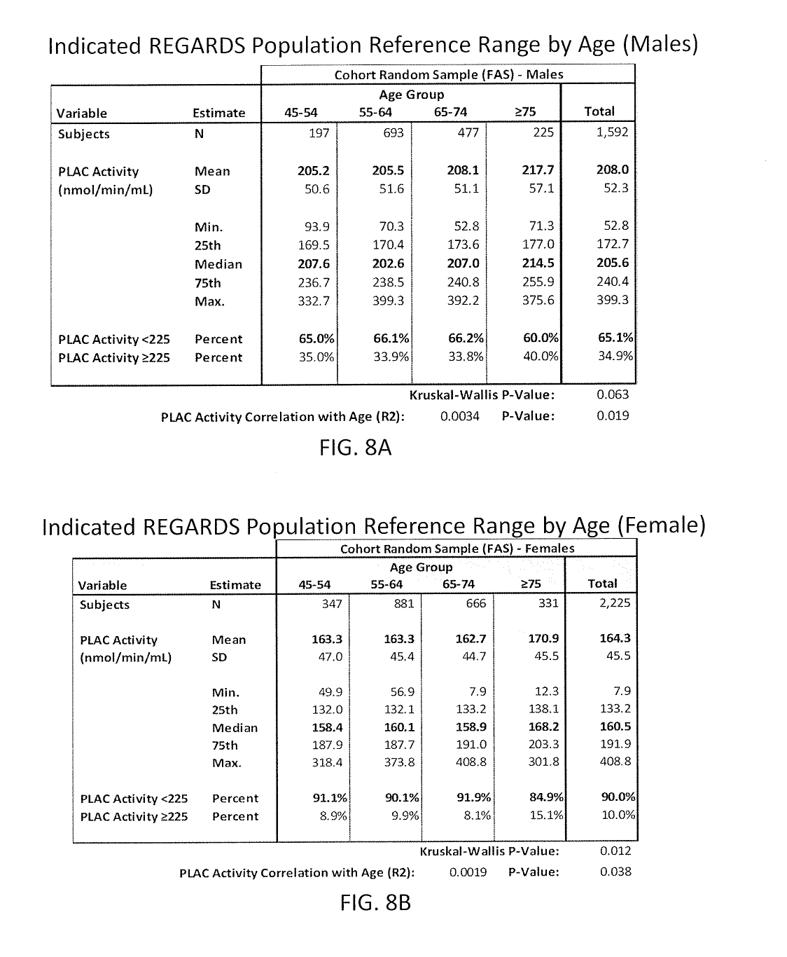

Following CLSI recommendations, the relationship of PLAC Activity value and age was also analyzed. PLAC Activity exhibited very consistent population value distributions across age decade categories. Very little correlation of PLAC Activity value with age was observed, with an R² of 0.003 (p<0.001) for the full CRS; such finding, consistent in each gender, was likely only significant in light of the large size of the CRS sample. These analyses are shown in FIG. 7 and FIGS. 8A and 8B (Indicated REGARDS Population Reference Range) which tabulates the value distributions (mean, standard deviation, inter-quartile range, median, min and max) of PLAC Activity in the Clinical Validation Study by age decade using the full CRS, as well as within each individual gender.

Following the efficacy, supportive and component endpoint analyses of this relationship, PLAC Activity population value distributions in the Clinical Validation Study CRS are further analyzed in the PLAC Activity Values by Subgroup section and in the Racial Diversity Study section.

Conclusions—Baseline Descriptive Statistics.

The Cohort Random Sample (CRS) selected for analysis is highly representative of the parent Indicated REGARDS Population, with no statistically significant differences in clinical characteristics observed.

The Case-Cohort Cases selected for analysis comprise the vast majority (>90%) of the total CHD cases within the parent Indicated REGARDS Population, and also similarly exhibit no statistically significant differences.

PLAC Activity, as measured in the CRS, had a median population value of 177.8 nmol/min/mL (IQR 145.0-215.5 nmol/min/mL).

The Analysis Cut point of 225 nmol/min/mL selected represents the 79.6% CRS population percentile.

The differing values and percentile positions of the Analysis Cut point in the various race and gender subgroups are broadly consistent with the subsequent rate of CHD events experienced by each such subgroup in the REGARDS study.

Primary and Secondary Efficacy Analyses (Cut Point Analyses).

Kaplan-Meier Log-Rank Test (Primary Analysis)

The overarching clinical use to be validated was that a pre-specified PLAC Activity cut point value, the Analysis Cut point of 225 nmol/min/mL, could be used as a binary classifier to categorize patients into two groups which would have a statistically significant difference in their absolute risk (probability) of subsequent CHD events.

The primary statistical hypothesis for this was that there was a statistically significant difference in the absolute risk (event rate) for the Primary Clinical Endpoint between the low PLAC Activity group (r₁) and the high PLAC Activity group (r₂) (H_{A}: r₁≠r₂), measured over the full available validation study duration. The null hypothesis was that both groups share the same absolute risk for events (H₀: r₁=r₂).

The primary statistical analysis employed was a weighted Kaplan-Meier survival analysis, which was performed in the full analysis set and separately for each race (FAS: Full Analysis Set, WAS: Caucasian and BAS: African American) and compared using a log-rank test (p≦0.05).

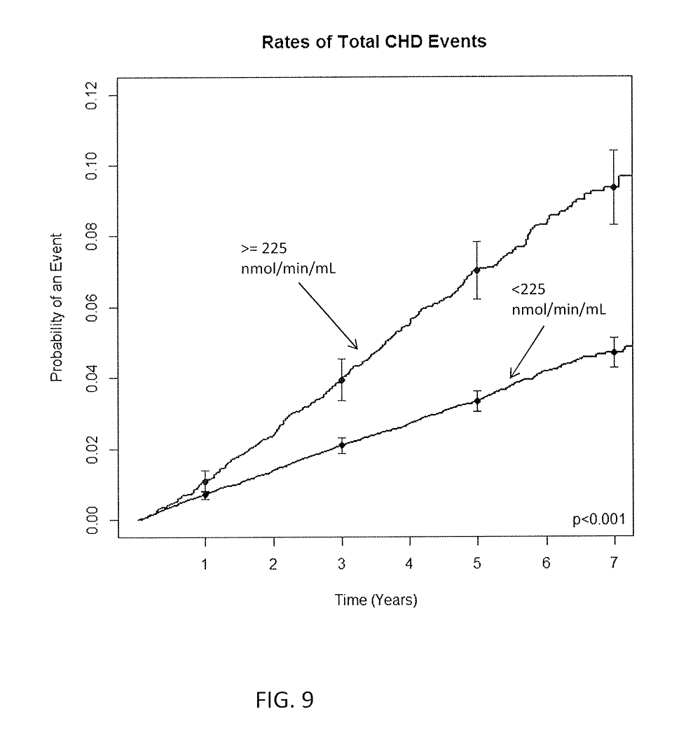

Graphical presentation of the Kaplan-Meier analysis of the PLAC Activity groups formed by the Analysis Cut point, providing absolute risk estimates for the Primary Clinical Endpoint in each group is shown in FIG. 9. Absolute risks (event rates) shown are calculated as one minus the event-free survival rate using the weighted Kaplan-Meier estimator (product limit estimator).

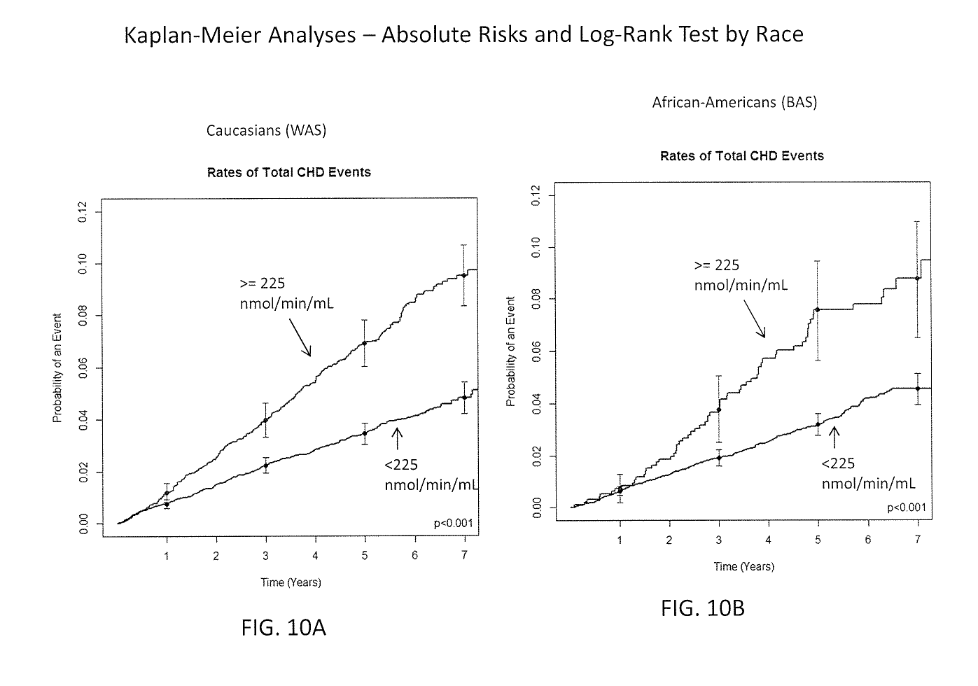

This analysis was also performed in each race individually, as shown in FIGS. 10A and 10B.

The pre-specified Clinical Validation Study success criteria were met for the primary efficacy analysis in the full Clinical Validation Study and within each race subpopulation, each achieving a Kaplan-Meier log-rank test p-value <0.001.

Pre-Test Absolute Risk of CHD Events.

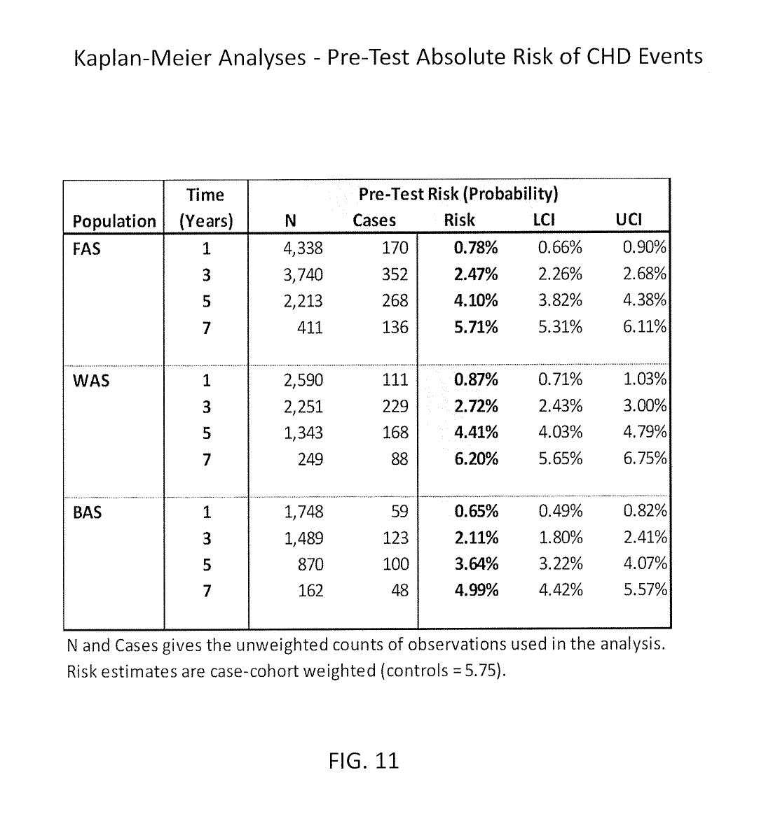

Using the weighted Kaplan-Meier estimates, FIG. 11 presents the pre-test risk (cumulative event rate) for the Primary Clinical Endpoint in the general study population, without regards to their PLAC Activity (i.e. both PLAC Activity groups combined). This, together with each associated 2-sided 95% confidence interval (95% CI), is calculated in each of the three analysis sets (FAS, WAS, and BAS) at the one, three, five, and seven year cumulative follow-up durations that were pre-specified in the SAP.

The pre-test event rate represents the best estimate of the absolute risk (probability) of CHD events for all individuals prior to their binary test classification into one of the two risk groups based on their PLAC Activity values. In the FAS, the general Clinical Validation Study population had a cumulative pre-test event rate of 4.1% [95% CI: 3.8%-4.4%] at five years follow-up (approximately the median follow-up duration of 5.3 years). Caucasian and African-American races individually had cumulative pre-test event rates to five years of 4.4% [4.0%-4.8%] and 3.6% [3.2%-4.1%], respectively, prior to binary classification into the appropriate PLAC Activity groups, indicating a modest difference in pre-test absolute risks between the race groups.

Post-Test Absolute Risk of CHD Events by PLAC Activity Group.

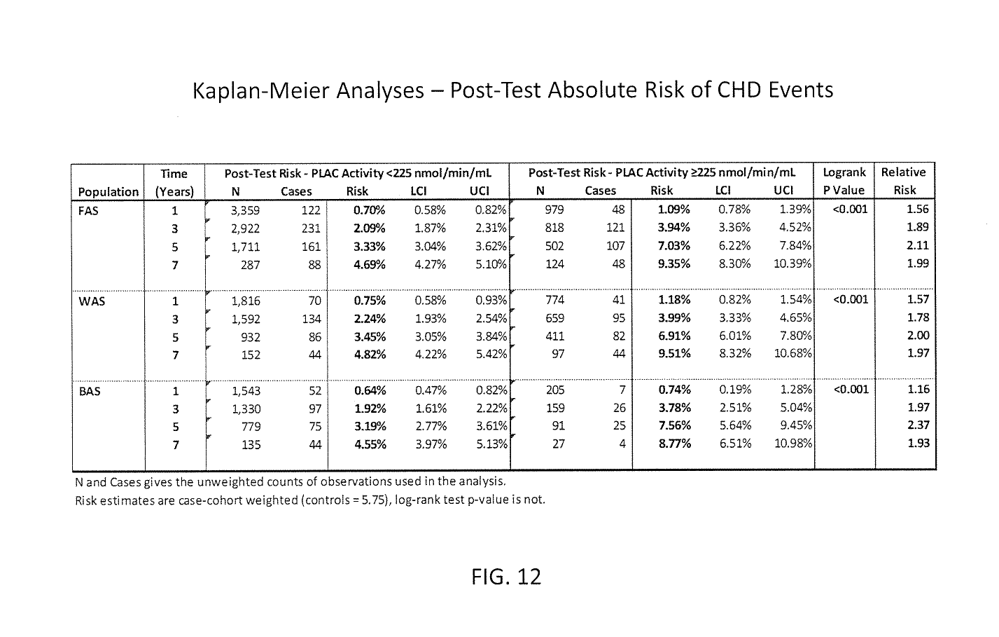

Using the previous Kaplan-Meier estimates, the post-test risks (cumulative event rates) for the Primary Clinical Endpoint within each PLAC Activity group were also tabulated within the three analysis sets (this is the same information as presented graphically in FIGS. 10A and 10B). This is presented for each analysis set in FIG. 12, together with each associated 2-sided 95% confidence interval (95% CI), relative risk, as well as the log-rank test p-value over the entire study duration that was the primary efficacy analysis.

The post-test event rate represents the best estimate of the absolute risk (probability) of CHD events after participants are classified into one of two groups based on their PLAC Activity values in relation to the Analysis Cut point. The analysis indicates that the absolute risks for the two PLAC Activity groups are distinctly separate, and in fact have 95% confidence intervals that do not overlap at any point from three years onwards in the Clinical Study. At the earlier one year duration, the point estimates of such absolute rates already differ, although the cumulative number of events at that time remains underpowered for comparisons of 95% CIs (see Sample Size and Power Assessment above). The above is true in both the full study analysis set (FAS) and in each of the Caucasian (WAS) and African-American (BAS) analysis sets taken individually. Regardless of the number of events at the one-year period in this Clinical Validation Study, a patient with a LpPLA2 activity level of greater than or equal to 225 nmol/min/mL is at increased risk of CHD within one year. The same is true at 2, 3, 4, 5, 6, and 7 years.

In the FAS, the high PLAC Activity group experienced 2.1 times the event rate of the low PLAC Activity group across the Clinical Validation Study at five years follow-up (approximately the median follow-up duration of 5.3 years). At this duration, the cumulative CHD event rate was 7.0% [6.2%-7.8%] for the high PLAC Activity group versus 3.3% [3.0%-3.6%] for the low PLAC Activity group. As shown above, each race subpopulation showed consistent results at this duration, with each high PLAC Activity group experiencing twice or greater the absolute risk of its low PLAC Activity group counterpart.

Post-test absolute risk for any given PLAC Activity group were shown to be very similar between the Caucasian and African-American race subpopulations. At every time point from three years onward, and within every PLAC Activity group, the 95% CI of the event rate for one race was shown overlap with and include the point estimate of the event rate for the other race, and vice versa. This provides further evidence that the races can be combined in study analyses. Further, this evidence teaches away from previous reports that Lp-PLA2 activity levels may need to be interpreted differently for various races.

Example of PLAC Activity Binary Classification for Risk Prediction.

Physicians commonly use likelihood ratios to translate diagnostic test results into predictions of post-test risk. Likelihood ratios are the ratios of the post-test odds to the pre-test odds (Decks & Altman 2004). Pre-test odds are calculated using the pre-test risk (prior probability, p₁) of an event occurring divided by the probability of it not occurring (pre-test odds (o₁)=p₁/[1−p₁]) for the individual to be tested (prior to knowledge of the individual's diagnostic test result). Then, after the individual's diagnostic test classification into a higher (test “positive”) or lower (test “negative”) risk group, the pre-test odds are multiplied by an appropriate result-specific likelihood ratio to arrive at a predicted post-test odds (post-test odds (o₂)=o₁×likelihood ratio). The predicted post-test risk (posterior probability, p₂) is then calculated by dividing these post-test odds by the sum of the odds plus one (post-test risk (p₂)=o₂/[o₂+1]).

When expressed as a likelihood ratio of the post- to pre-test odds for CHD events over the subsequent five years, the above Clinical Validation Study results indicate that the high PLAC Activity group had post-test odds which were 1.77 times the pre-test odds of the general unclassified population (a positive likelihood ratio or “LR+” of 1.77). The low PLAC Activity group had post-test odds which were 0.81 times these same pre-test odds (a negative likelihood ratio or “LR−” of 0.81).

In clinical use, a physician would estimate a patient's pre-test risk and convert it to a pre-test odds. This would then be multiplied by the appropriate Clinical Validation Study likelihood ratio given above in order to arrive at a post-test odds, which then could be converted into the patient's post-test absolute risk.

Using the overall Clinical Validation Study population statistics as an individual example, a typical participant would have the general population pre-test absolute risk of 4.1% (0.043 odds) for CHD events over the subsequent five years (0.8% per year). If this individual tested into the high PLAC activity group (representing 20% of the Indicated REGARDS Population), using the positive likelihood ratio would result in an increased risk prediction of 7.0% post-test absolute risk (0.076 odds) over the same period (1.4% event risk per year). Likewise, if this individual tested into the low PLAC activity group (representing 80% of the Indicated REGARDS Population), using the negative likelihood ratio would result in a decreased prediction of 3.3% post-test absolute risk (0.035 odds) over the same period (0.7% event risk per year).

These differences in post-test risk are particularly important when viewed in light of the continuing lifetime nature of CHD risk. Using common cardiovascular risk categories, such prediction of increased risk would result in the high PLAC Activity group individual being re-categorized into a higher risk category, at % event risk per year (≧10% ten-year risk). The low PLAC Activity group individual would be confirmed as remaining in the lower risk category of <1% event risk per year (<10% ten-year risk).

This difference in likelihood ratios and absolute post-test risks of subsequent CHD in the Clinical Validation Study represents a clinically important demonstration of the predictive power of binary classification using the PLAC Activity Test at the Analysis Cut point. Risk assessments for individual patients would mirror the use of other traditional cardiovascular risk biomarkers, placing the results in context with other clinical and demographic indicators (e.g. age, gender, smoking, etc.) and with traditional cardiovascular risk factors (e.g. low density lipoprotein, total cholesterol, etc.) to refine their pre-test risk assessments and improve post-test risk predictions and recommendations in routine primary prevention.

Within the individual race analysis sets, the above calculations are also clinically significant, with statistically overlapping post-test absolute event rates for each PLAC Activity group across the races and consistent conclusions for individual PLAC Activity group post-test risk versus the general population pre-test risk. This is particularly notable in light of the pre-test risk differences previously presented for each race, and again provides further evidence that the races can be combined in study analyses.

Conclusions—Primary Efficacy Analysis

The pre-specified PLAC Activity Analysis Cut point of 225 nmol/min/mL was shown to be a statistically significant predictor for CHD events, classifying the Clinical Validation Study population into two PLAC Activity groups with statistically different absolute rates of CHD events.

The primary efficacy analysis achieved all pre-specified success criteria, with a Kaplan-Meier analysis log-rank p-value <0.001

The confidence intervals of the absolute risk (event rates) of the two PLAC Activity groups were statistically separated as soon as sufficient events had accrued for power, and consistently remained separate for the remaining study duration.

At five year follow-up, the high PLAC Activity group had over twice the absolute rate for CHD events seen in the low PLAC Activity group with the following cumulative absolute rates of CHD events, including 95% CIs:

High PLAC Activity group: 7.0% [6.2%-7.8%]

Low PLAC Activity group: 3.3% [3.0%-3.6%]

The likelihood ratios and resulting post-test absolute event rates were demonstrated to represent a clinically important difference in absolute risk of CHD events for the high PLAC Activity group.

Positive Likelihood Ratio (LR+)=1.77 (High PLAC Activity group)

Negative Likelihood Ratio (LR−)=0.81 (Low PLAC Activity group)

All of the above findings were also confirmed within each race individually (Caucasians and African-American). At five year follow-up, the high PLAC Activity group had an increased absolute rate of events over the low PLAC Activity group as follows:

For Caucasians, the low and high PLAC Activity group experienced 3.5% [3.1%-3.8%] and 6.9% [6.0%-7.8%] event rates, respectively (2.0 times relative risk).

For African-Americans, the low and high PLAC Activity group experienced 3.2% [2.8%-3.6%] and 7.6% [5.6%-9.5%] event rates, respectively (2.4 times relative risk).

The two races were thus also consistent in both their post-test absolute event rates and relative risks, further supporting their combination in full study analyses.

Cox Proportional Hazards Ratio (Secondary Analysis)

Following the success of the primary clinical efficacy analysis, a secondary efficacy analysis was performed to compare the ratio of the hazard rates of the two PLAC Activity groups.

The secondary statistical hypothesis was that there was a statistically higher hazard ratio (“FIR”) for Primary Clinical Endpoint events in the high PLAC Activity group over the low PLAC Activity group. Weighted Cox proportional hazards regression models were employed in each such population to estimate the relative risk for total CHD events in the high PLAC Activity group relative to the low PLAC Activity group, as estimated by the hazard ratio derived from the model's beta coefficient (H_{A}: HR>1.0). The null hypothesis was that the low PLAC Activity group and the high PLAC Activity group had the same relative risk (H₀: HR=1.0).

High PLAC Activity was to be determined to be an independent predictor of events if (a) the high PLAC Activity group membership term point estimate of the HR exceeded 1.0, and (b) the 95% CI of the HR did not overlap 1.0.

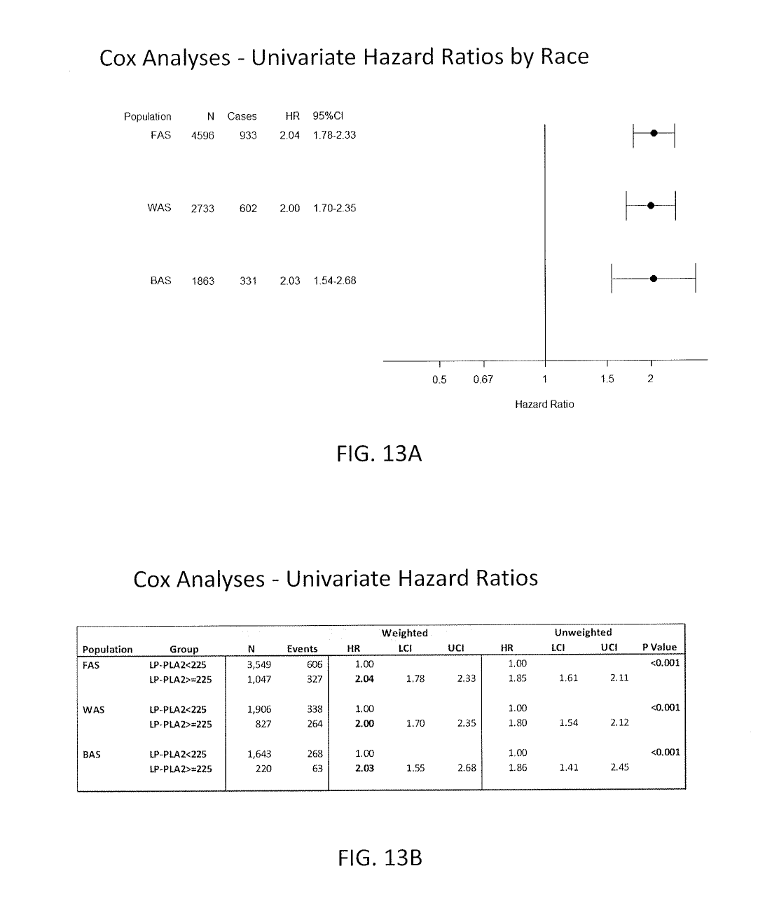

Using the appropriate population, a weighted univariate Cox proportional hazards regression was performed with high PLAC Activity group binary classifier as the sole term in the model (versus low PLAC Activity group as the baseline hazard with HR=1.0). Weighted case-cohort hazard ratios with associated 95% CIs are graphically presented in FIG. 13A for each analysis set (FAS, WAS, and BAS), together with the actual subject and case counts used in each such analysis.

The observed subject counts N shown are reduced by 2 subjects, from the full 4,598 subjects in the Clinical Validation Study descriptive analyses to 4,596 usable in efficacy analyses; these 2 control subjects do not have complete follow-up times allowing time to event analyses, but were selected in the CRS, tested for PLAC Activity, and are used elsewhere in descriptive statistics.

The pre-specified Clinical Validation Study success criteria were again achieved in the secondary efficacy analyses for the full study (FAS) and all race subpopulations (WAS, BAS), with each achieving a statistically significant FIR.

The above results are also tabulated in FIG. 13B, which further includes the actual subject and case counts used by each PLAC Activity group and unweighted Cox model results, prior to the application of case-cohort weights.

The hazard ratios were very similar between Caucasians and African-Americans, with the point estimates of hazard ratios within one race virtually identical to the other, and each HR point estimate within the 95% CI of the other race. This provides further evidence that the races can be combined in subsequent study analyses.

Furthermore, the parallel unweighted analyses indicate that the statistical significance of the Clinical Validation Study conclusions are not impacted by the use of case-cohort weights, which provide a more accurate estimate of the underlying prevalent event rates and hazard ratios by adjusting for the prior probabilities within the full Indicated REGARDS Population. The raw unweighted hazard ratios of the high PLAC activity group remained greater than one and statistically significant in every analysis set (FAS, WAS, and BAS).

Conclusions—Secondary Efficacy Analysis

In the Clinical Validation Study, the high PLAC Activity group created by the Analysis Cut point of 225 nmol/min/mL was shown to have a statistically higher relative hazard for CHD events than that of the low PLAC Activity group of <225 nmol/min/mL.

The secondary efficacy analysis achieved all pre-specified success criteria, with a Cox proportional hazard ratio of 2.04 [95% CI 1.78-2.33, p<0.001].

All of the above findings were also confirmed within both Caucasian and African-American races analyzed independently, with HRs of 2.00 [1.70-2.35, p<0.001] and 2.08 [1.55-2.68, p<0.001], respectively.

The consistent HRs and overlapping confidence intervals within the two races further support their combination in full study analyses.

Parallel unweighted Cox hazards analysis were also consistently significant, indicating that the above conclusions were independent of the use of case-cohort weights adjusting the case-cohort study design to reflect the prior probabilities of the full parent Indicated REGARDS Population.

PLAC Activity Group by Race Interaction Test

In assessing the impact of combining the individual Caucasian (WAS) and African-American (BAS) analysis sets in subsequent analyses, Cox proportional hazards regression models were used to test for a potential formal statistical interaction of PLAC Activity group by race in the prediction of the Primary Clinical Endpoint (CHD events), using the combined full analysis set (FAS) of the Clinical Validation Study. This test was performed at a significance level of 0.05 (p<0.05).

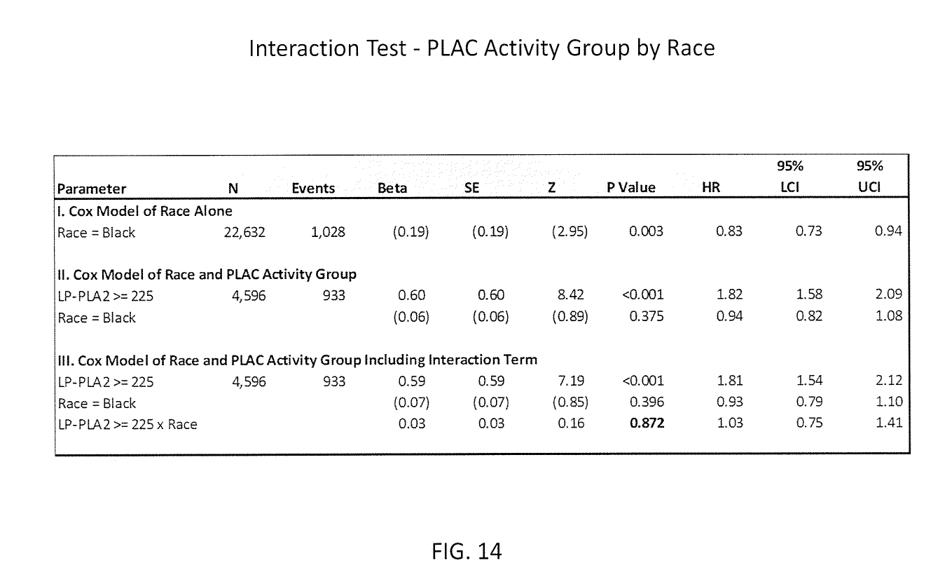

Specifically, such interaction tests are designed to detect any potential moderation relationship between PLAC Activity prediction of CHD events and race, where the predictive power of PLAC Activity would depend upon race, as might be hypothesized given the population differences in PLAC Activity values observed by race. The results of this analysis, which is performed without case-cohort weighting for comparability between the three Cox models used, is presented in FIG. 14.

In the first Cox model presented, African-American race was shown to be a statistically significant predictor of subsequent CHD events in a univariate Cox model of race as the sole variable, with a race hazard ratio (HR) of 0.83 [0.73-0.94; p=0.003] of African-Americans (relative to Caucasians as the baseline hazard), using the full power of the entire Indicated REGARDS Population dataset (as PLAC Activity group is not required in such univariate analysis). This is unsurprising given the previously described difference in event rates by race in the full Indicated REGARDS Population, with CHD events experienced by 4.91% of enrolled Caucasians participants versus 3.85% of enrolled African-Americans participants through the Dec. 31, 2010 adjudication date; in other words, African-Americans have a lower hazard rate of CHD events relative to Caucasians (see FIG. 3).

However, in the second Cox model, adjusting the first model for high PLAC Activity group membership (versus low PLAC activity group as the baseline hazard), African-American race no longer remains significant as a predictor of CHD events in the Clinical Validation Study full analysis set (FAS), with an unweighted HR of 0.94 (0.82-1.08; p=0.375). In contrast, high PLAC Activity group membership, relative to low PLAC Activity group membership, is shown to be a significant predictor of CHD events, with an unweighted HR of 1.82 [1.58-2.09, p<0.001] in the bivariate model.

The third Cox model shown further adjusted the second model with an additional interaction term for PLAC Activity group by race, that term being the product of two binary categorical variables (high PLAC Activity group membership times African-American race), designed to affect any zero-order correlation between such variables. This formal interaction model demonstrates that there is not a statistical moderation interaction between the predictive power of high PLAC Activity group membership and race. In this third Cox model, high PLAC Activity group membership remained a statistically significant predictor of CHD events, with an HR of 1.81 [1.54 2.12, p<0.001] that was effectively unchanged from the bivariate model. Neither race nor the formal interaction term achieves statistical significance as predictors; the HR of the formal interaction term was not significant at 1.03 [0.75-1.41, p=0.872].

This last model demonstrates that there is not a statistical interaction, and that the predictive power of PLAC Activity group does not depends on race. This was further confirmed in supportive Cox efficacy analyses within each race analysis set (WAS, BAS) taken independently, which indicated substantially equivalent HRs for PLAC Activity group between each race (see Subgroup Analyses by Race section).

Conclusions—PLAC Activity by Race Interaction Test

Using Cox model analysis, no statistically significant interaction of PLAC Activity group by race was detected for the prediction of CHD events (p=0.872).

PLAC Activity group was unchanged in its prediction of CHD events upon addition of an interaction term to Cox models (moderation analysis).

High PLAC Activity group remained significant as a predictor, with a hazard ratio (HR) of 1.81 [1.54-2.12, p<0.001] for CHD events relative to low PLAC Activity group as the baseline hazard.

The interaction term (High PLAC Activity×Race) was not statistically significant as a predictor, with an HR of 1.03 [0.75-1.41, p=0.872]

This interaction analysis further supports the combination of the two races in subsequent analyses.

Other Supportive Efficacy Analyses (Cut Point Analyses)

The use of the different analysis sets in other subsequent supportive analyses of the marker and the racial differences in marker performance was subject to the results from the previous interaction test of PLAC Activity group by race performed in the full analysis set population (FAS). As no statistical interaction of race by PLAC Activity group was observed, subsequent supportive analyses are generally presented in the FAS, combining both races together, unless specifically noted otherwise.

Nonetheless, African-American race is included as a covariate in all risk-adjusted Cox models using the FAS, and the full parallel supportive Cox analyses by race, in the African-American BAS and Caucasian WAS analysis sets individually, is presented within the subgroup analyses (see Subgroup Analyses by Race below).

Multivariate Cox Proportional Hazards Models.

Three additional multivariate Cox proportional hazards regression models were performed to further describe the risk-adjusted predictive power of PLAC Activity group categorization for the Primary Clinical Endpoint (total CHD composite). These expanded the previous Cox regression model (See Cox Proportional Hazards Ratio (Secondary Analysis) above), by controlling for other covariates of event risk.

As in the prior analysis, the Analysis Cut point was used in the binary categorization of subjects into the high and low PLAC Activity groups using their individual PLAC Activity values, and PLAC Activity is expressed in the Cox models as a binary categorical variable for each subject's high PLAC Activity group membership (with low PLAC Activity group membership representing the baseline hazard).

The following multivariate Cox proportional hazards regression models were used:

Model 1: Demographically-Adjusted Cox Model.

A first “demographically-adjusted model” adjusted predictive power by including the covariates of age (continuous), race (Caucasian/African-American), and gender (male/female) with PLAC Activity Group membership. These covariates represent demographic variables controlled for in the REGARDS parent study design.

Model 2: Risk-Adjusted Cox Model.

A second “risk-adjusted model” further adjusted for diabetes (yes/no), hypertension (yes/no), and current smoking (yes/no). These added factors represent the most common primary prevention risk markers which are not directly associated with the lipoprotein and lipid metabolism related to Lp-PLA₂ activity.

Model 3: Extensively Risk-Adjusted Cox Model.

A third “extensively risk-adjusted model” further adjusted for high-density lipoprotein (HDL, expressed as a binary variable with cut point=40 mg/dL for males and 50 mg/dL for females) and low-density lipoprotein (LDL, expressed as a binary variable with cut point at 130 mg/dL), utilizing cut points published in public health guidelines for treatment in lower risk categories (National Cholesterol Education Program 2002). These added primary prevention risk markers are known from past studies to be structurally associated with the Lp-PLA₂ enzyme, which has a biological mechanism related to both of these lipoprotein types (Thompson et al. 2010). As a result, their addition to Lp-PLA₂ Activity may represent an overcorrection in multivariate regression modeling.

Methods were otherwise as used previously in the secondary efficacy analysis. High PLAC Activity was determined to be an independent predictor of events if (a) the high PLAC Activity group membership term point estimate of the HR exceeded 1.0, and (b) the 95% CI of the HR did not overlap 1.0.

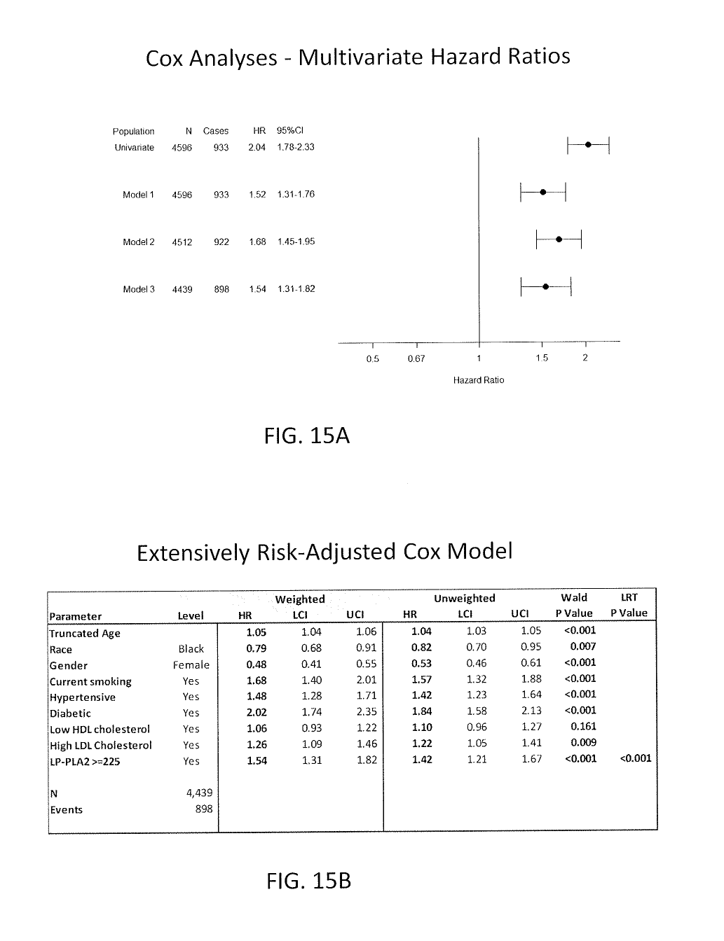

The three models, together with the univariate Cox model of the previous secondary efficacy analysis, are summarized graphically in FIG. 15A. For each of the three models, the high PLAC Activity group HR (relative to the low PLAC Activity group as the baseline hazard) and its associated 95% CI are reported (after case cohort weights), together with the actual counts of case-cohort observations used in such analysis.

As graphically presented in FIG. 15A, the Clinical Validation Study success criteria pre-specified for the secondary efficacy analysis univariate Cox model were again achieved in each multivariate Cox model, with each achieving a statistically significant HR for the high PLAC Activity group relative to the low PLAC Activity group.

The full results for the extensively risk-adjusted Cox model (Model 3) are also tabulated in FIG. 15B, which further includes the HR and associated 95% CIs and p-values for each covariate, together with the same for unweighted Cox model results, prior to the application of case-cohort weights.

As previously noted, all success criteria of the Clinical Validation Study were met. For the high PLAC Activity group hazard ratio, an additional p-value was also calculated using the likelihood-ratio test (“LRT”) statistic, and presented above together with the study p-values (calculated using Wald statistics). Identical conclusions were reached.

Of particular note in this full multivariate model was that high PLAC Activity versus low PLAC Activity proved a greater hazard for subsequent CHD events than many established cardiovascular risk factors, notably hypertension, HDL, and LDL cholesterol covariates, which were dichotomized using published practice guidelines for such risk factors (National Cholesterol Education Program 2002).

African American race returned to significance as a covariate (HR=0.79 [0.68-0.91], p=0.007) after this extensive risk-adjustment, suggesting that race may inform CHD event risk in a more complex manner, possibly only in reference to the other covariates; race is notably absent as a variable in any of the previously described risk calculators used in primary prevention. An additional confirmatory model was constructed adding the previous high PLAC Activity by race interaction term to Model 3 (see PLAC Activity Group by Race Interaction Test section above); this additional term was found not to be statistically significant in the full model (HR=1.04 [0.75-1.43], p=0.5167), with the conclusions and HR estimates of high PLAC Activity and all of the above covariates remaining unchanged by the its inclusion.

Conclusions—Multivariate Cox Proportional Hazards Models

Using Cox proportional hazards models, high PLAC Activity (defined by the Analysis Cut point) was shown to remain a statistically significant predictor of CHD events after full risk adjustment for other cardiovascular risk factors.

These factors included age, race, gender, smoking, hypertension, diabetes, LDL and HDL, used in the models per published practice guidelines.

After full risk-adjustment for these factors, the Cox hazard ratio of the high PLAC Activity group was 1.54 [95% CI 1.31-1.82, p<0.001] relative to the low PLAC Activity group.

The multivariate Cox analyses indicate a consistent predictive power of PLAC Activity group membership across a variety of clinical characteristics.

Parallel unweighted analyses were also consistently significant, indicating robust consistency regardless of the application of case-cohort weights.

Conclusions were not changed by the addition of a PLAC by race interaction term to the full model, which again proved not significant (interaction term HR=1.04 [0.75-1.43], p=0.5167).

The results of the multivariate Cox proportional hazards models are consistent with, and supportive of, the successful primary and secondary efficacy analyses.

Subgroup Analyses by Gender

The Kaplan-Meier absolute risk estimates and Cox models used to establish the clinical efficacy of the PLAC Activity group binary classification (Cut point Analyses) as a predictor of subsequent CHD events were repeated individually within each gender subgroup, using the above (i) univariate, (ii) demographically-adjusted, (iii) risk-adjusted, and (iv) extensively risk-adjusted Cox models as previously, adjusted appropriately given the subgroup under analysis (i.e. removing gender as a covariate in Cox analyses within a single gender).

Kaplan-Meier Efficacy Analyses.

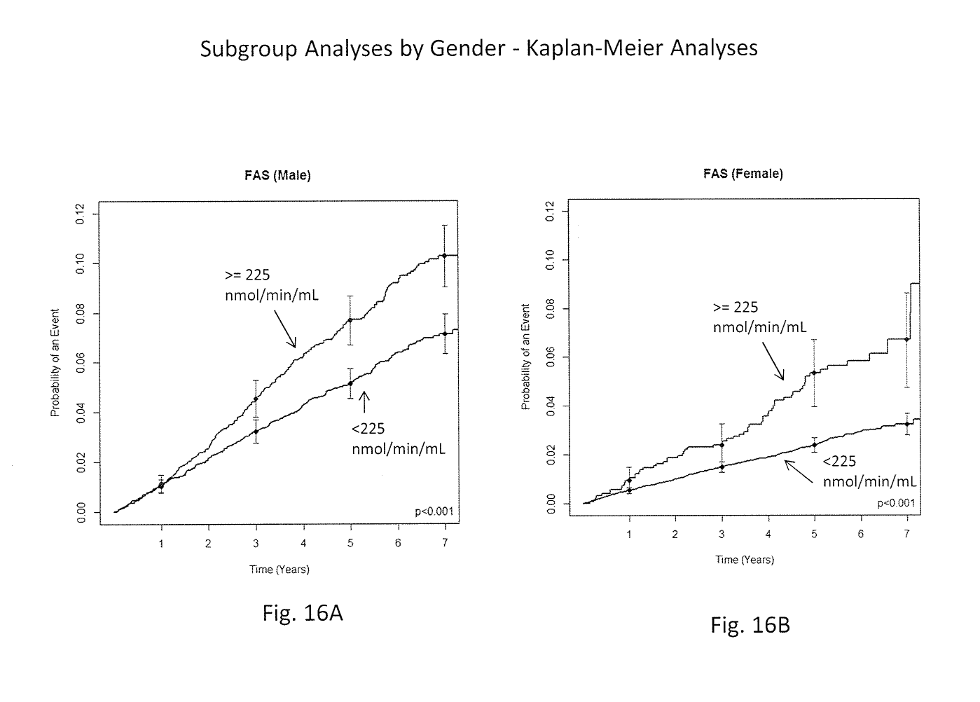

The graphical Kaplan-Meier analyses by gender are presented in FIGS. 16A (males) and 16B (females). Methods are as used previously in the primary efficacy analysis.

The Clinical Validation Study success criteria were again achieved, with each gender subgroup achieving a statistically significant log-rank test for an absolute event rate difference high PLAC Activity group versus the low PLAC Activity group through the duration of the study (p<0.001 within each gender). Similar to the full study, the 95% confidence intervals of the absolute event rates for the two PLAC Activity groups do not overlap in either gender once sufficient events have accrued (by the third and fifth year of follow-up onwards, for males and females, respectively); the point estimates of such rates are already consistently different at the first year of follow-up.

At five year follow-up (approximately the median follow-up of 5.3 years), the low and high PLAC Activity group's absolute rate of CHD events in each gender was as follows. For males, the low and high PLAC Activity group experienced 5.2% [4.6%-5.8%] and 7.7% [6.7%-8.7%] event rates, respectively, representing a 1.5 times relative risk of high to low PLAC Activity group membership. For females, the low and high PLAC Activity group experienced 2.4% [2.1%-2.7%] and 5.3% [3.9%-6.7%] event rates, respectively, representing a 2.2 times relative risk of high to low PLAC Activity group membership.

Given the differences in PLAC value distributions by gender, female high PLAC Activity group membership indicated a more selected group of high-risk females (10.0% of total female subjects in the CRS) at an increased absolute risk of CHD events approaching that experienced by the more prevalent male high PLAC Activity group (34.9% of male subjects in the CRS).

Cox Proportional Hazards Efficacy Analyses.

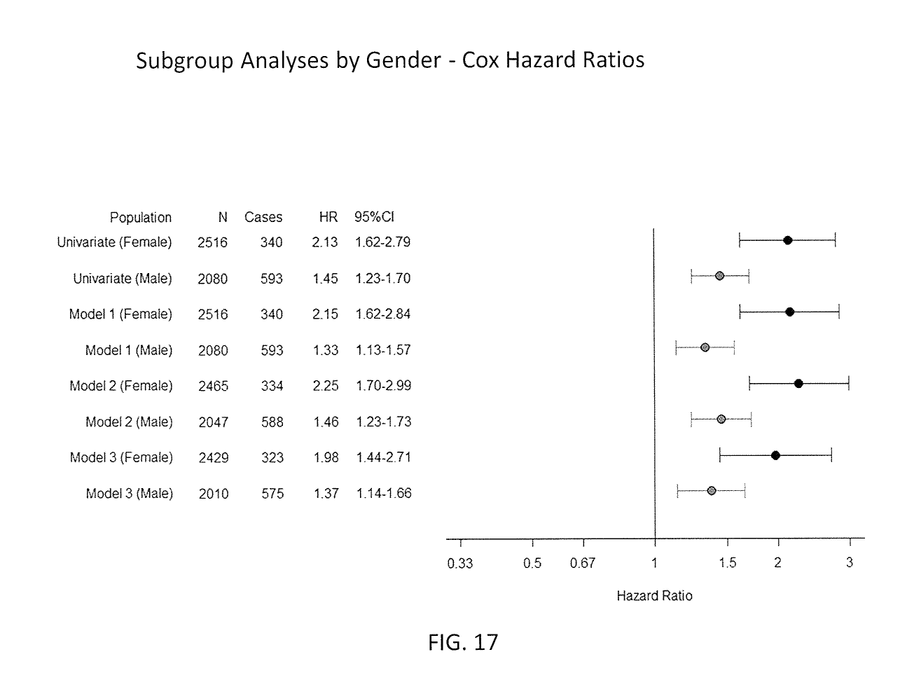

The completed by gender subgroup Cox analyses resulted in a total of eight Cox model comparisons, being the univariate Cox model and the three multivariate Cox models for each of the two genders, which are presented together in FIG. 17. The Cox hazard ratios and associated 95% CIs are reported for the high PLAC Activity group binary classification variable within each gender subgroup and graphically presented together, allowing comparisons between males and females.

The Clinical Validation Study success criteria were again achieved in each gender taken individually, with every Cox model achieving a statistically significant HR for the high PLAC Activity group relative to the low PLAC Activity group. Consistent with the previous absolute event rate observations, the hazard ratios for high PLAC Activity membership within the females appears to be higher than within the males; however, the confidence intervals of the individual gender hazard ratios appear to overlap more with successive risk adjustment.

For the extensively risk-adjusted Cox model (Model 3), the point estimate of high PLAC Activity group proportional hazards ratio for females was 45% higher than that of males, with females reporting an HR of 1.98 [1.44-2.71, p<0.001] and males reporting an HR of 1.37 [1.14-1.66, p=0.018]. The difference in proportional hazards by gender did not indicate a statistically significant quantitative interaction (FDA 2011) after risk adjustment. This was tested in a similar Cox model performed in the full analysis set (FAS), using the same extensively risk-adjusted covariates together with gender and an interaction term (the product of two binary categorical variables for high PLAC Activity and Female Gender), the interaction term was found not to be statistically significant, reporting an HR of 1.23 [0.90-1.70, p=0.154].

Regardless of the generally increased hazards for high PLAC Activity group membership in females relative to males, all conclusions within each individual gender analyzed independently remained statistically significant, and consistent directionally both between the genders and with the study as a whole. In both females and males, the high PLAC Activity group created by the Analysis Cut point of 225 nmol/min/mL was shown to have a statistically higher relative hazard for CHD events than that of the low PLAC Activity group of <225 nmol/min/mL.

Conclusions—Subgroup Analyses by Gender

In both males and females, membership in the high PLAC Activity group defined by the Analysis Cut point of ≧0.225 nmol/min/mL was shown to be a statistically significant predictor of increased risk of CHD events relative to the low PLAC Activity group of <225 nmol/min/mL.

All Clinical Validation Study success criteria for the full study were also statistically significant within both individual genders.

Kaplan-Meier analyses indicated a statistically significant difference in the absolute rates of CHD events between the PLAC Activity groups, with a log-rank p-value of <0.001 in both males and females.

At five year follow-up, the high PLAC Activity group had an increased absolute rate of events over the low PLAC Activity group as follows:

For males, the low and high PLAC Activity group experienced 5.2% [4.6%-5.8%] and 7.7% [6.7%-8.7%] event rates, respectively (1.5 times relative risk).

For females, the low and high PLAC Activity group experienced 2.4% [2.1%-2.7%] and 5.3% [3.9%-6.7%] event rates, respectively (2.2 times relative risk).

Similarly, after extensive risk-adjustment, the high PLAC Activity group Cox hazards ratio for females was 45% higher than that of males, with males reporting an HR of 1.37 [1.14-1.66, p=0.018] and females reporting an HR of 1.98 [1.44-2.71, p<0.001].

Tests for a quantitative interaction of high PLAC Activity by gender in the full study were not statistically significant (p=0.154).

The results of the subgroup analyses by gender are consistent with, and supportive of the successful efficacy analyses of the full study.

Subgroup Analyses by Race

The Kaplan-Meier absolute risk estimates and Cox models used to establish the clinical efficacy of PLAC Activity group binary classification (Cut point Analyses) as a predictor of subsequent CHD events were repeated individually within each race analysis set (WAS and BAS), using the above (i) univariate, (ii) demographically-adjusted, (iii) risk-adjusted, and (iv) extensively risk-adjusted Cox models adjusted appropriately given the subgroup under analysis (i.e. removing race as a covariate in Cox analyses within a single race).

Kaplan-Meier Efficacy Analyses.

The graphical and tabular Kaplan-Meier primary efficacy analyses of absolute risk for each race were previously presented and discussed in the Kaplan-Meier Log-Rank Test (Primary Analysis) section above. All success criteria of the Clinical Validation Study were also achieved in each of the individual race subgroups analyzed independently.

The two races were consistent in both their post-test absolute event rates and relative risks, as previously described. At five year follow-up, in both races, the high PLAC Activity group had an increased absolute rate of events over the low PLAC Activity group. For Caucasians, the low and high PLAC Activity group experienced 3.5% [3.1%-3.8%] and 6.9% [6.0%-7.8%] event rates, respectively (2.0 times relative risk). For African-Americans, the low and high PLAC Activity group experienced 3.2% [2.8%-3.6%] and 7.6% [5.6%-9.5%] event rates, respectively (2.4 times relative risk).

Cox Proportional Hazards Efficacy Analyses.

Similarly, the univariate Cox proportional hazards secondary efficacy analyses of hazard ratios model race were previously presented and discussed in the Cox Proportional Hazards Ratio (Secondary Analysis) section above. All success criteria of the Clinical Validation Study were also achieved in each of the individual race subgroups analyzed independently.

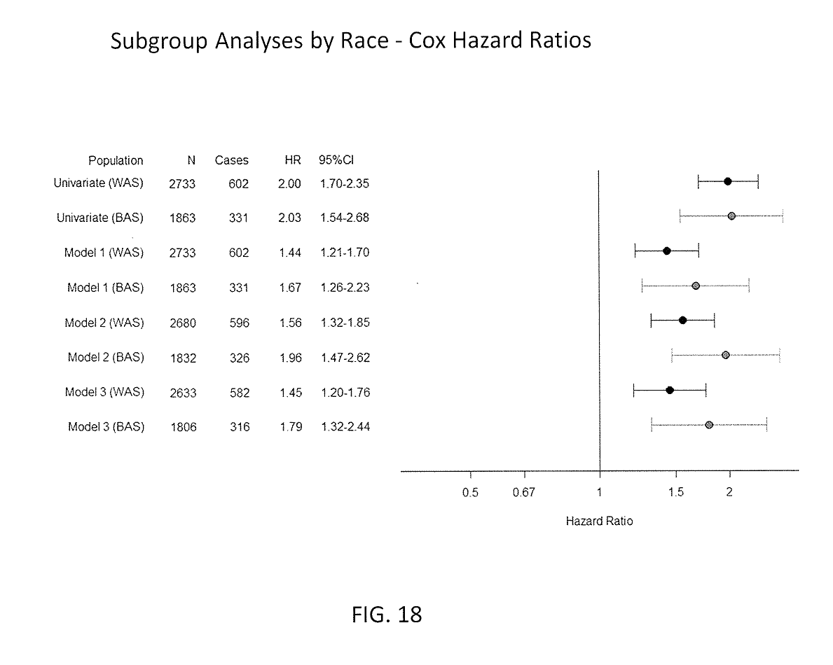

Multivariate Cox model analyses were additionally performed, adjusting for risk factors as previously described. The completed by race subgroup Cox analyses resulted in a total of eight Cox model comparisons, the univariate Cox model (repeated here for comparability) and the three multivariate Cox models for each of the two races, which are presented together in FIG. 18. The Cox hazard ratios and associated 95% Cis are reported for the high PLAC Activity binary classification variable within each race subgroup and graphically presented together, allowing comparisons between the Caucasian WAS and African-American BAS.

The Clinical Validation Study success criteria were again achieved in each race taken individually, with every Cox model in either race achieving a statistically significant HR for the high PLAC Activity group relative to the low PLAC Activity group.

As in the previous univariate Cox models, the hazard ratios for high PLAC Activity membership within one race were generally consistent with those within the other race given the relative power of each analysis set; for each Cox model, the point estimates of the hazard ratio for the better-powered Caucasian WAS were always within the 95% confidence interval of the same hazard ratio for the lesser-powered African-American BAS.

Nonetheless, the point estimates of the hazard ratios within the African-American BAS were consistently higher than the same analyses within the Caucasian WAS in each model. For the extensively risk-adjusted Cox model (Model 3), the point estimate of the high PLAC Activity group proportional hazards ratio for African-Americans was 23% higher than that of Caucasians, with Caucasians reporting an HR of 1.45 [1.20-1.76, p=0.005] and African-Americans reporting an HR of 1.79 [1.32-2.44, p=0.001].

A further analysis indicated the differences in risk-adjusted proportional hazards ratio by race also did not indicate a statistically significant quantitative interaction (FDA 2011). This was tested with a similar Cox model performed in the full analysis set (FAS), using the same extensively risk-adjusted covariates together with gender and an interaction term (the product of two binary categorical variables for high PLAC Activity and African-American race), the interaction term was again found not to be statistically significant, reporting an HR of 1.04 [0.75-1.43, p=0.569].

Regardless of the possibly increased hazards for high PLAC Activity group membership in African-Americans relative to Caucasians, the conclusions within each individual race analyzed independently remained statistically significant, and consistent directionally both between the races and with the study as a whole. In both Caucasians and African-Americans, the high PLAC Activity group created by the Analysis Cut point of ≧225 nmol/min/mL was shown to have a statistically higher relative hazard for CHD events than that of the low PLAC Activity group of <225 nmol/min/mL.

Conclusions—Subgroup Analyses by Race

In both African-Americans and Caucasians, membership in the high PLAC Activity group, defined by the Analysis Cut point of ≧225 nmol/min/mL was shown to be a statistically significant predictor of increased risk of CHD events relative to the low PLAC Activity group of <225 nmol/min/mL.

All Clinical Validation Study success criteria for the full study were also statistically significant within both individual races.

Kaplan-Meier analyses indicated a statistically significant difference in the absolute rates of CHD events between the PLAC Activity groups, with a log-rank p-value of <0.001 in both African-Americans and Caucasians.

The two races were also consistent in both their post-test absolute event rates and relative risks, as previously described. At five year follow-up, the high PLAC Activity group had an increased absolute rate of events over the low PLAC Activity group as follows:

For Caucasians, the low and high PLAC Activity group experienced 3.5% [3.1%-3.8%] and 6.9% [6.0%-7.8%] event rates, respectively (2.0 times relative risk).

For African-Americans, the low and high PLAC Activity group experienced 3.2% [2.8%-3.6%] and 7.6% [5.6%-9.5%] event rates, respectively (2.4 times relative risk).

Similarly, after extensive risk-adjustment, the high PLAC Activity group Cox hazards ratio for African-Americans was 23% higher than that of Caucasians, with Caucasians reporting an HR of 1.45 [1.20-1.76, p=0.005] and African-Americans reporting an HR of 1.79 [1.32-2.44, p=0.001].

Tests for a quantitative interaction of high PLAC Activity by race in the full study were not statistically significant (p=0.569).

The results of the subgroup analyses by race are consistent with, and supportive of, the successful efficacy analyses of the full study.

Secondary Component Endpoints (Cut Point Analyses)

CHD Components Versus Total CHD Composite

Overview.

The primary and secondary analyses, including absolute risk estimates and Cox models, were repeated as additional supportive analyses using each of the components of the Primary Clinical Endpoint (a composite of total CHD events) individually as the outcome of interest, i.e. separately by (a) myocardial infarction (MI), (b) coronary revascularization (Revasc), (c) CHD-related death (CHD Death). Each of these outcomes is referred to here as a Secondary Clinical Endpoint.

All analyses were performed within the full analysis set (FAS) to maximize power to detect differences in PLAC Activity prediction by outcome. Total subject and case counts are given for each analysis. Case-cohort subjects available in component analyses are reduced from the total used in composite CHD analyses by any composite CHD cases outside of the CRS who did not also experience the component outcome of interest.

Component Versus Composite Outcome Hazard Ratios.

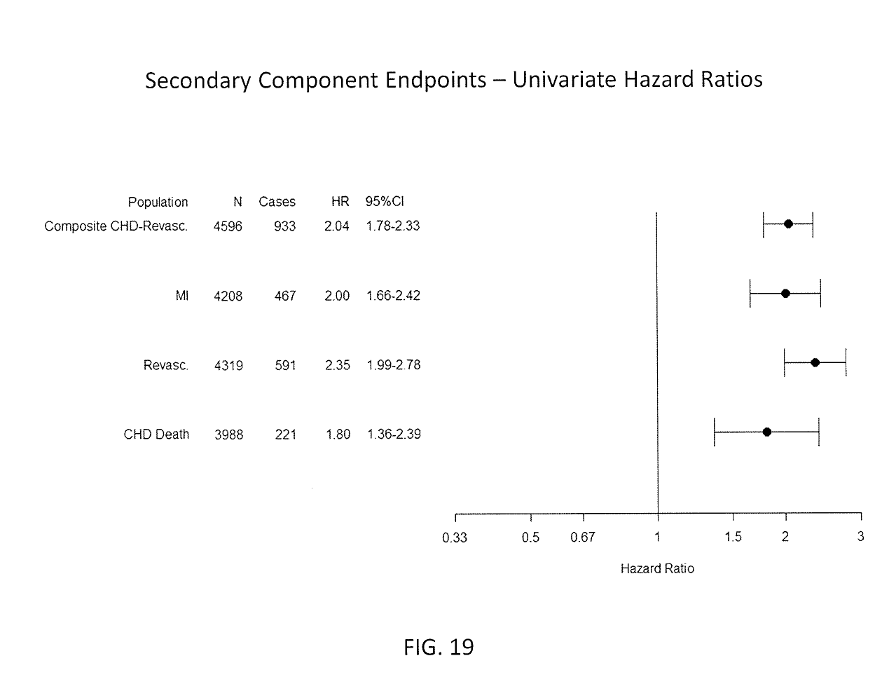

FIG. 19 presents a summary of the univariate Cox models (with the high PLAC Activity group binary classifier as the sole term) using each of the component Secondary Clinical Endpoints individually in place of the composite Primary Clinical Endpoint. The Cox hazard ratios and associated 95% CIs are reported for the high PLAC Activity group variable using each component Secondary Clinical Endpoint as outcome, and graphically presented together with the same univariate model using the composite Primary Clinical Endpoint (from the Secondary Efficacy Analysis), allowing comparisons between the component and composite results.

The Clinical Validation Study success criteria were again achieved using each component Secondary Clinical Endpoint individually as outcome in univariate Cox models, with each outcome model achieving a statistically significant HR for the high PLAC Activity group relative to the low PLAC Activity group (p<0.001 for every outcome model HRs).

All of the Secondary Clinical Endpoint outcome HRs were directionally consistent with the Primary Clinical Endpoint and significantly greater than one. The 95% confidence intervals of the component outcome HRs were substantially overlapping, with the width of such intervals consistent with the statistical power for each outcome (i.e., the number of cases experiencing each Secondary Component Endpoint of interest).

The HR point estimate using the total CHD composite Primary Clinical Endpoint (HR=2.04 [1.78-2.33], p<0.001) was contained within the confidence interval of each of the individual component endpoints. There was no evidence of any individual Secondary Component Endpoint dominating the composite Primary Clinical Endpoint, nor evidence of any individual Secondary Component Endpoint in which the PLAC Activity Analysis Cut point lacked statistically significant predictive power.

Weighted multivariate Cox proportional hazards regressions, using each Secondary Clinical Endpoint individually as the outcome of interest, were also performed using previous methods for (i) univariate, (ii) demographically-adjusted, (iii) risk-adjusted, and (iv) extensively risk-adjusted Cox models (not shown). Results were consistent, with high PLAC Activity group membership remained a statistically significant predictor for each Secondary Component Endpoint after risk adjustment.

Each individual Secondary Clinical Endpoints is subsequently explored below using Kaplan-Meier analyses in its own section.

Myocardial Infarction

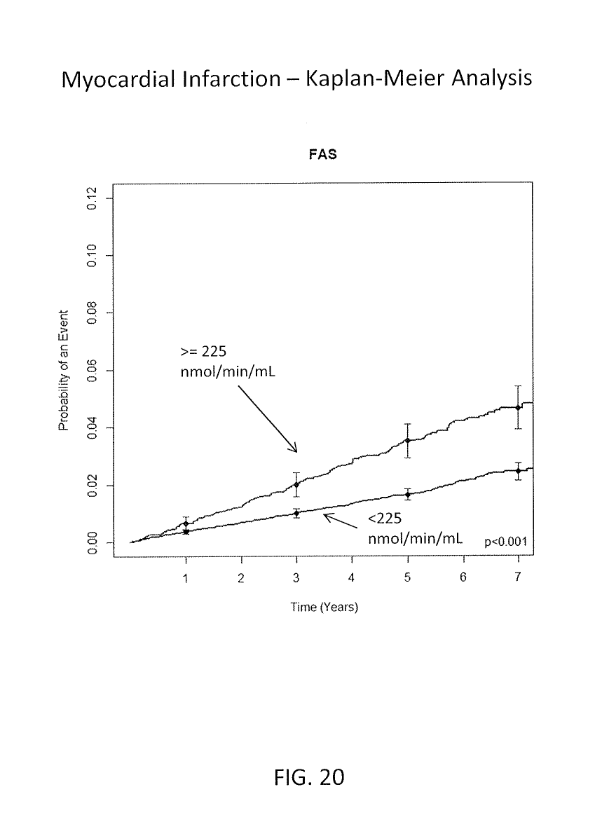

Graphical presentation of the Kaplan-Meier analysis of the PLAC Activity groups formed by the Analysis Cut point, using myocardial infarctions as the outcome events of interest, giving absolute risk estimates for the Primary Clinical Endpoint in each group, is provided in FIG. 20. Methods used otherwise followed the primary efficacy analysis.

The Clinical Validation Study success criteria, used in the primary efficacy analysis of the Primary Clinical Endpoint, were also met using myocardial infarction as the outcome of interest, with a Kaplan-Meier log-rank test p-value <0.001.

Coronary Revascularization

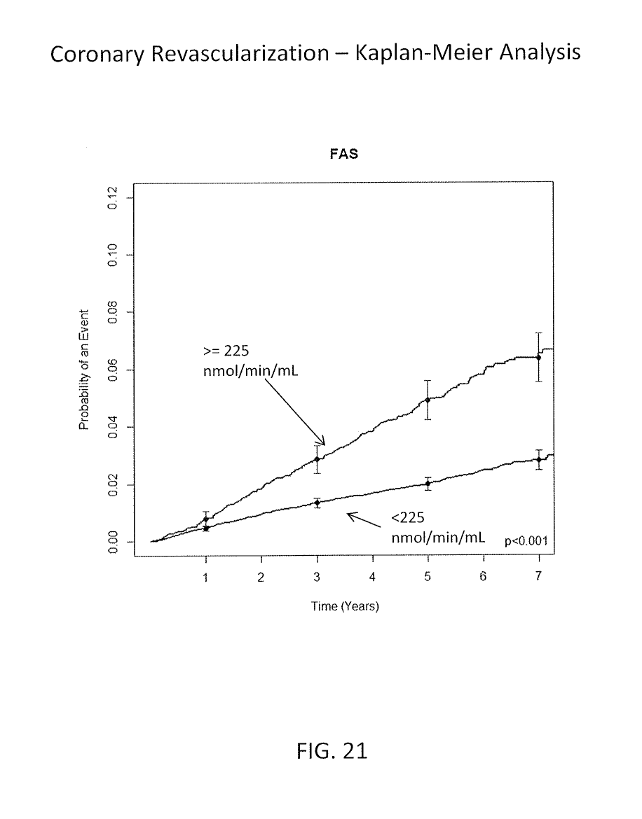

Graphical presentation of the Kaplan-Meier analysis of the PLAC Activity groups formed by the Analysis Cut point, using coronary revascularization procedures as the outcome events of interest, giving absolute risk estimates for the Primary Clinical Endpoint in each group, is provided in FIG. 21. Methods used otherwise followed the primary efficacy analysis.

The Clinical Validation Study success criteria, used in the primary efficacy analysis of the Primary Clinical Endpoint, were also met using coronary revascularization as the outcome of interest, with a Kaplan-Meier log-rank test p-value <0.001.

CHD-Related Death

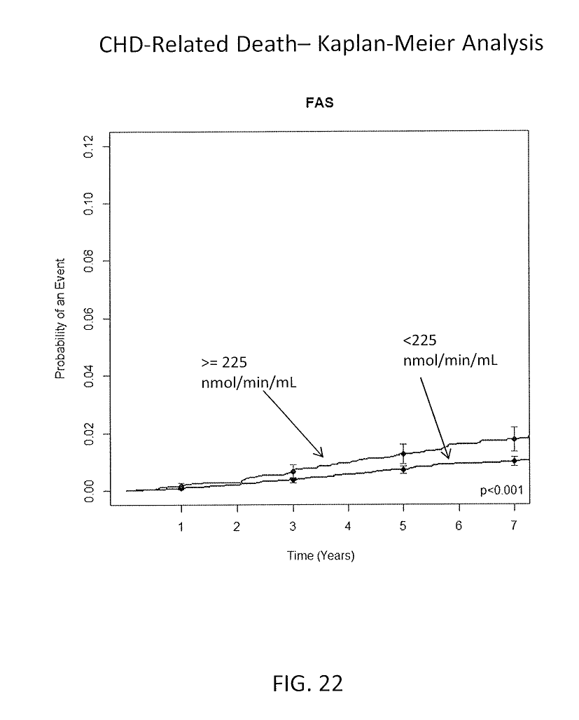

Graphical presentation of the Kaplan-Meier analysis of the PLAC Activity groups formed by the Analysis Cut point, using CHD-related deaths as the outcome events of interest, giving absolute risk estimates for the Primary Clinical Endpoint in each group, is provided in FIG. 22. Methods used otherwise followed the primary efficacy analysis.

The Clinical Validation Study success criteria, used in the primary efficacy analysis of the Primary Clinical Endpoint, were also met using CHD-related death as the outcome of interest, with a Kaplan-Meier log-rank test p-value <0.001.

Conclusions—Secondary Component Endpoints

Using Kaplan Meier and Cox proportional hazards regression as previously, high PLAC Activity (defined by the Analysis Cut point) was shown to be a statistically significant predictor of each Secondary Component Endpoint (CHD component event type) contained within the Primary Clinical Endpoint (total CHD composite), when such was analyzed as independent outcomes of interest.

The Secondary Component Endpoints analyzed were Myocardial Infarction, Coronary Revascularization, and CHD-Related Death.

Each Secondary Component Endpoint, analyzed independently, achieved the same Clinical Validation Study success criteria as used in the main efficacy analyses of the Primary Clinical Endpoint.

Kaplan-Meier analyses indicated a statistically significant difference between the PLAC Activity groups in the absolute event rates for each Secondary Component Endpoint, with a log-rank p-value of <0.001 in every outcome.

High PLAC Activity group membership was also a statistically significant predictor (HR>1.0, p<0.001) in univariate Cox models for each Secondary Component Endpoint as the sole outcome of interest.

High PLAC Activity group membership remained a statistically significant predictor for each Secondary Component Endpoint after risk adjustment.

Each Secondary Component Endpoint yielded a hazard ratio consistent with that of the Primary Clinical Endpoint. There was no evidence of any single dominant component endpoint, nor of any component in which PLAC Activity lacked significant predictive power.

The results of the Secondary Component Endpoint models are consistent with, and supportive of, the main efficacy analyses.

PLAC Activity Tertile and Continuous Analyses

The following pre-specified analyses of the SAP again utilize the Primary Clinical Endpoint (total CHD composite) as the event of interest in defining cases, and assesses the predictive power of PLAC Activity across values other than the Analysis Cut point, providing supportive evidence across the range of PLAC Activity measurements.

Population-defined PLAC Activity tertiles were analyzed using Kaplan-Meier analysis as previously. As a further pre-specified parallel efficacy analysis, continuous (log₂ transformed) PLAC Activity value was analyzed in Cox models as a continuous log₂ transformed variable, indicating the increase in proportional hazard for CHD events across the entire range of PLAC Activity values observed in the Clinical Validation Study.

PLAC Activity Tertile Analyses

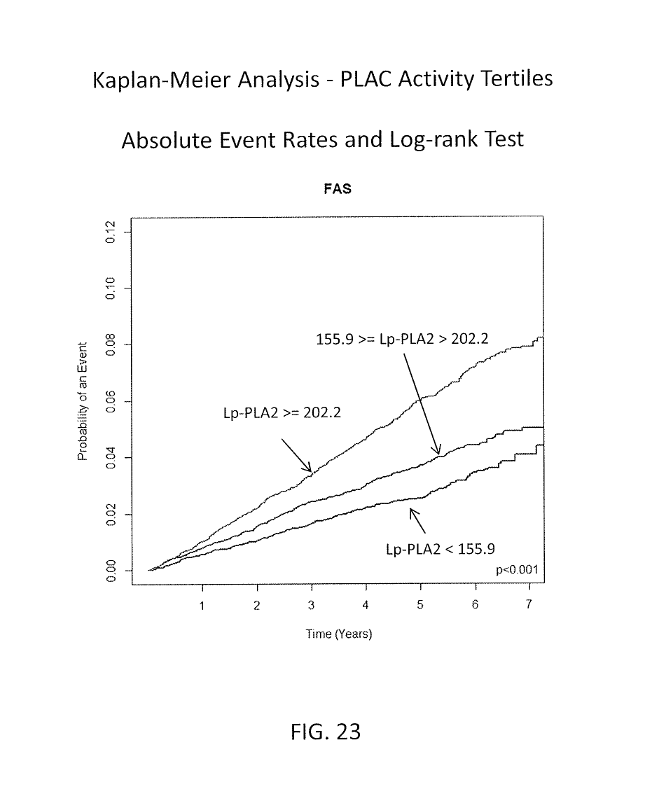

Using the Cohort Random Sample reference intervals as the unbiased direct random sample of the overall parent Indicated REGARDS Population, PLAC Activity tertile group limits were defined across the full Clinical Validation Study (FAS), and weighted Kaplan Meier analyses performed as in the primary efficacy analysis, using log-rank tests at a 0.05 significance level (p<0.05). The absolute event rates and log-rank test result within each PLAC Activity tertile group are presented graphically in FIG. 23.

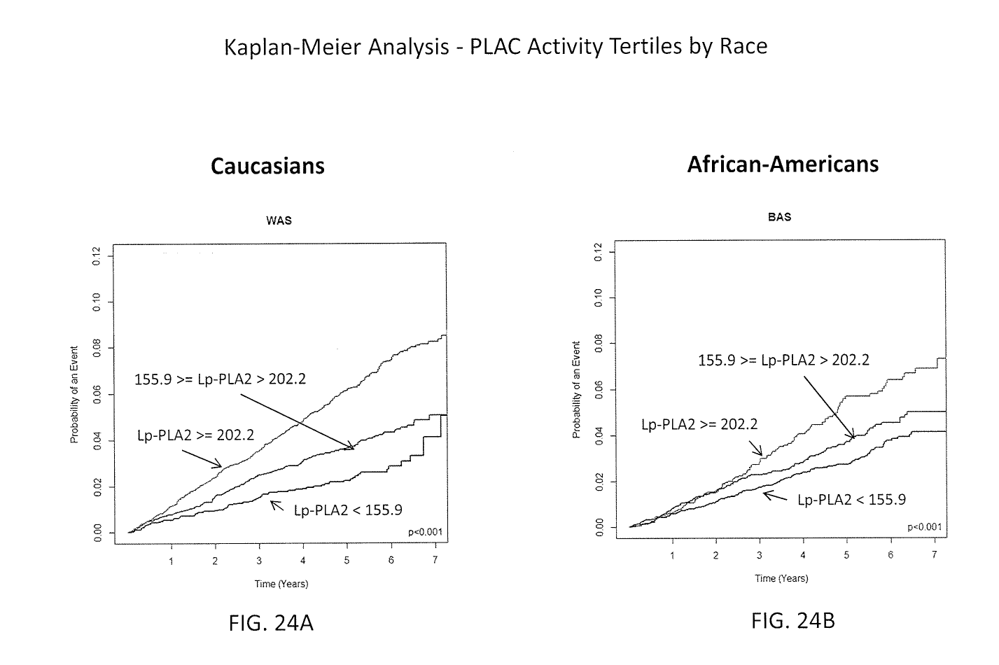

The same analysis was also performed in each race analysis set individually, as shown in FIGS. 24A and 24B. The same fixed PLAC Activity values defining the tertile divisions within the FAS) were also used unchanged across both race analysis sets (WAS, BAS), further validating the transportability of common cut points across the races.

As presented graphically above, the Clinical Validation Study success criteria pre-specified for the primary efficacy analyses was also achieved using PLAC Activity tertile groups, both within the full study (FAS) and within each by race analysis set (WAS, BAS) individually. Each analysis achieved a statistically significant log-rank test for an absolute event rate differences across the consistent PLAC Activity tertile value groups analyzed through the duration of the study (p<0.001 for both total study and within each race).

PLAC Activity Continuous Analyses

Continuous PLAC Activity values were also used directly as pre-specified in the SAP for supportive efficacy analyses. In such analyses, the PLAC Activity values were log transformed (log₂) in accordance with the SAP, where each one unit increase in the transformed value represents a doubling of the untransformed PLAC Activity value.

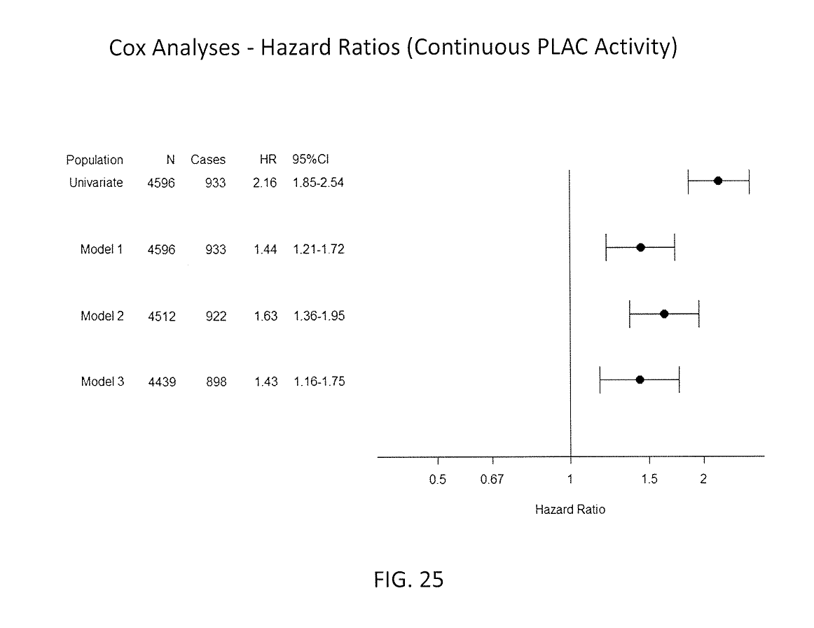

Weighted Cox proportional hazards regressions, using the Primary Clinical Endpoint (total CHD composite) as the outcome of interest, were performed using previous methods for (i) univariate, (ii) demographically-adjusted, (iii) risk-adjusted, and (iv) extensively risk-adjusted Cox models. The Cox hazard ratios and associated 95% CIs are reported for the log₂-transformed PLAC Activity value and graphically presented together in FIG. 25, allowing comparisons at the different levels of risk-adjustment.

As shown above, the Clinical Validation Study success criteria pre-specified for the secondary efficacy analyses (cut point analyses using univariate Cox models) was also achieved in each continuous PLAC Activity value Cox model, with log₂ transformed PLAC Activity values achieving hazard ratios for subsequent CHD events that maintained statistical significance through additional risk-adjustment in every model.

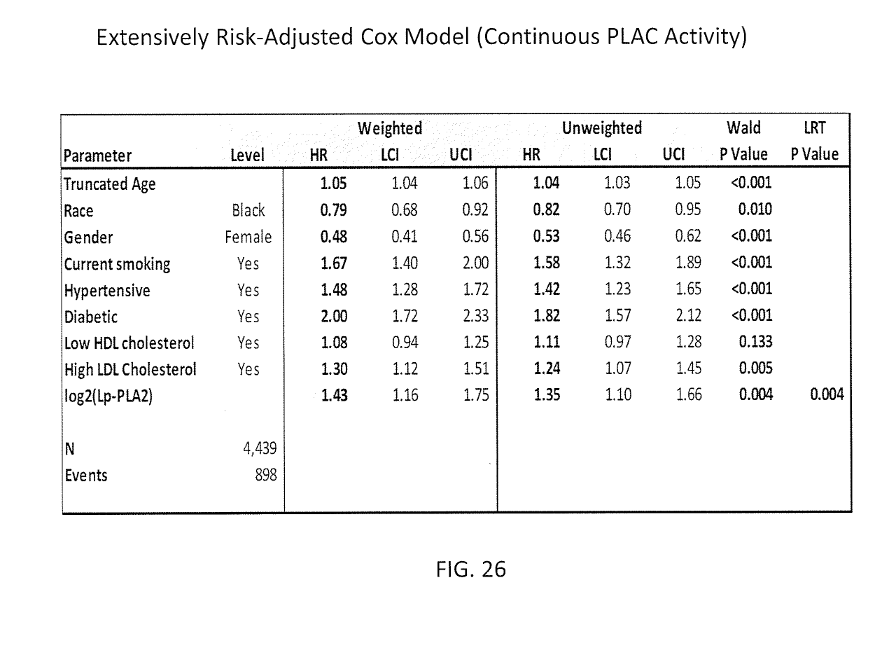

The full results for the extensively risk-adjusted Cox model (Model 3) are also tabulated in FIG. 26, which further includes the HR and associated 95% CIs and p-values for each covariate, together with unweighted Cox model results, prior to the application of case-cohort weights.

As previously noted, all success criteria of the Clinical Validation Study were met in this supportive analysis. For the continuous PLAC Activity hazard ratio, an additional p-value was also calculated using as previously using the LRT statistic and above together with the study p-values (calculated using Wald statistics). Identical conclusions were reached.

As previously, neither race nor gender exhibited a statistically significant interaction with continuous PLAC Activity values after risk adjustment. Continuous interaction testing was performed using previously described methods adding a continuous PLAC Activity interaction term (for each subject, the product of the continuous log₂ transformed PLAC Activity value and the binary covariate of interest) to Model 3 for race and gender (two separate models). In the by race interaction test, the continuous PLAC Activity by race interaction term reported an HR of 0.74 [0.53-1.05, p=0.180]. In the by gender interaction test, the continuous PLAC Activity by gender interaction term reported an HR of 1.09 [0.77-1.55, p=0.515].

Conclusions—PLAC Activity Tertile and Continuous Analyses

Using Kaplan Meier and Cox proportional hazards regression analyses, increases in PLAC Activity value were shown to be statistically significant predictors of the Primary Clinical Endpoint (total CHD composite) across PLAC Activity values other than the Analysis Cut point.

Analyses were performed over PLAC Activity population tertile groups and also continuously over the reported range of PLAC Activity values.

PLAC Activity tertile groups were shown by Kaplan-Meier analysis to exhibit increasing absolute rates of CHD events, which differed by log-rank test (p<0.001 for the full study and within each race individually).

Continuous PLAC Activity values (log 2 transformed) were shown to have a statistically significant hazard ratio for CHD events using Cox proportional hazards regression (univariate HR=2.16 [1.85-2.54], p<0.001).

Continuous PLAC Activity values remained statistically significant after extensive risk-adjustment with the full set of covariates (HR=1.43 [1.16-1.75], p=0.004).

Continuous PLAC Activity values exhibited no statistically significant interaction by race (p=0.180) or by gender (p=0.515) after such risk-adjustment.

The results of the above analyses indicate PLAC Activity values other than the Analysis Cut point provide consistent prediction supportive of the main efficacy analyses.

PLAC Activity Values by Subgroup

Gender, Race and Age (Reference Range Analyses)

An analysis of PLAC Activity population values by race and gender was previously presented in FIG. 6 (see Baseline Descriptive Statistics section). Population values are further analyzed by age decade in FIG. 7 and gender in FIGS. 8A and 8B (Indicated REGARDS Population Reference Range).

Further analyses of the Clinical Validation Study PLAC Activity value distributions are also presented by race with comparison to other races and independent reference intervals in the Racial Diversity Study section below.

Outcome

The following analyses utilize the Primary Clinical Endpoint (total CHD event composite) as the definition of cases.

In the Clinical Validation Study, descriptive analyses of the PLAC Activity value distribution was also performed in each of the following outcome subgroups:

Cohort Random Sample (CRS sample of Parent Indicated REGARDS Population);

Subjects not experiencing the Primary Clinical Endpoint (CRS Controls);

Subjects experiencing the Primary Clinical Endpoint (Case-Cohort Cases).

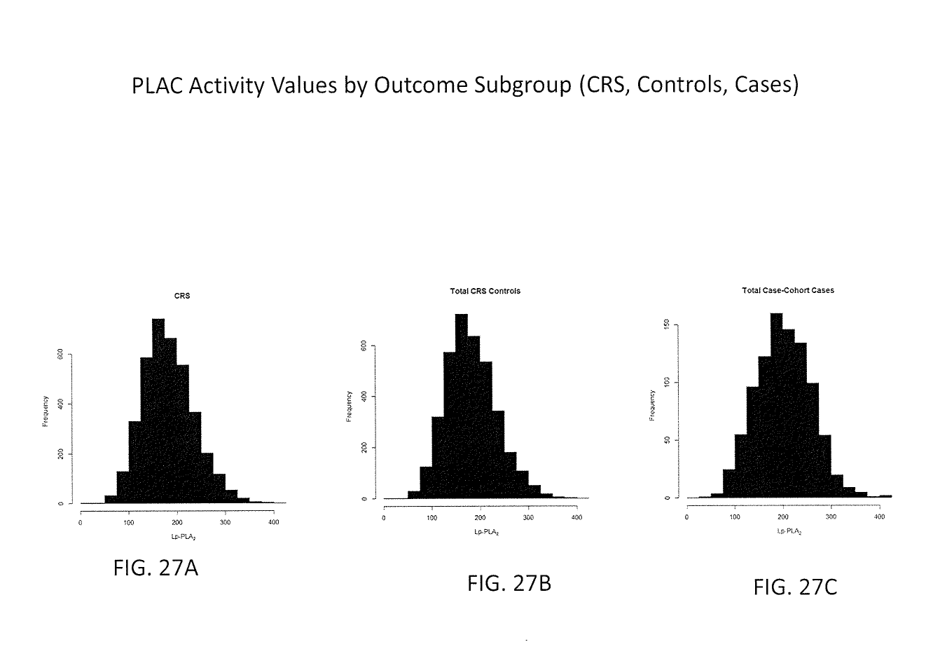

Each of these is initially presented as a histogram for the total CRS (FAS) in FIGS. 27A, 27B and 27C.

PLAC Activity in the full CRS ranged from a minimum of 7.9 to a maximum of 408.8 nmol/min/mL, with a median value of 177.8 nmol/min/mL (IQR 145.0-215.5 nmol/min/mL). The PLAC Activity Analysis Cut point of 225 nmol/min/mL represents the 79.6^{th} CRS population percentile in the CRS.

As would be expected given the predictive power of PLAC Activity, case-cohort cases exhibited higher PLAC Activity values versus CRS controls, with median values of 200.3 nmol/min/mL (IQR 162.7-242.7 nmol/min/mL) and 177.1 nmol/min/mL (IQR 144.4-213.7 nmol/min/mL).

Race and Outcome

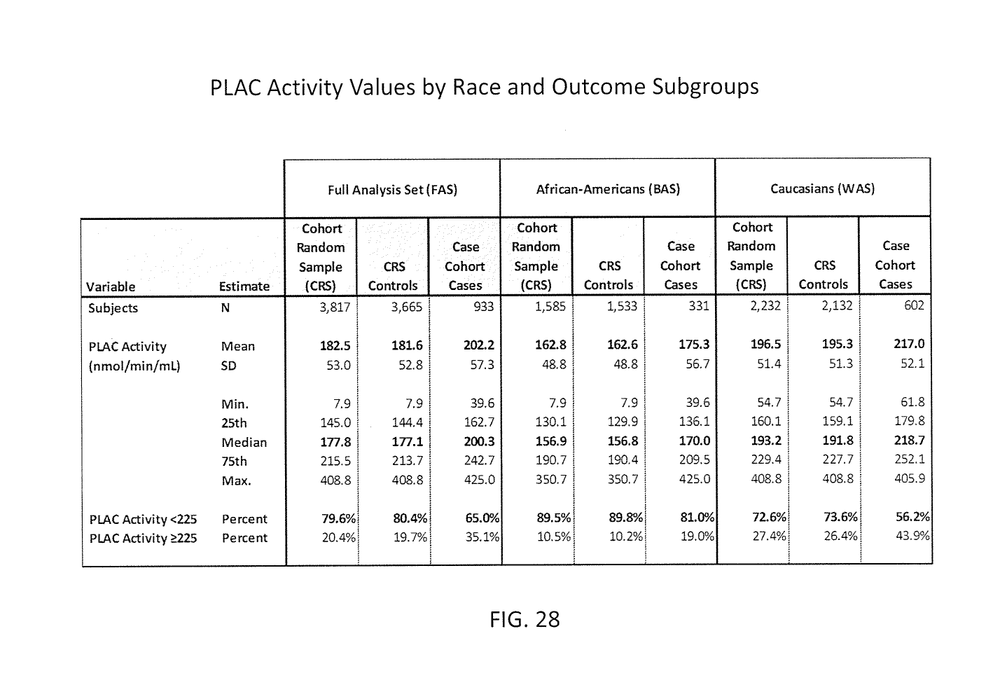

FIG. 28 tabulates the value distributions (mean, standard deviation, inter-quartile range, median, min and max) of PLAC Activity in the Clinical Validation Study by the same outcome subgroups as shown above, for the full case-cohort (FAS), as well as within each individual race analysis set (BAS and WAS). The non-parametric percentile position represented by the Analysis Cut point is also shown, giving the percentages of each given population below (the low PLAC Activity group) and at or above (the high PLAC Activity group) the cut point.

Although Caucasians exhibit higher values than African-Americans, with a median PLAC Activity value of 193.2 (IQR 160.1-229.4) versus 156.9 (IQR 130.1-190.7) nmol/min/mL, respectively, a substantial overlap of interquartile ranges is seen between each race. In both races, cases exhibited higher PLAC Activity values than controls.

The Analysis Cut point represents the 89.5^{th} percentile in the BAS (African American CRS population) and the 72.6^{th} CRS population percentile in the WAS (Caucasian CRS population).

Race, Gender and Outcome

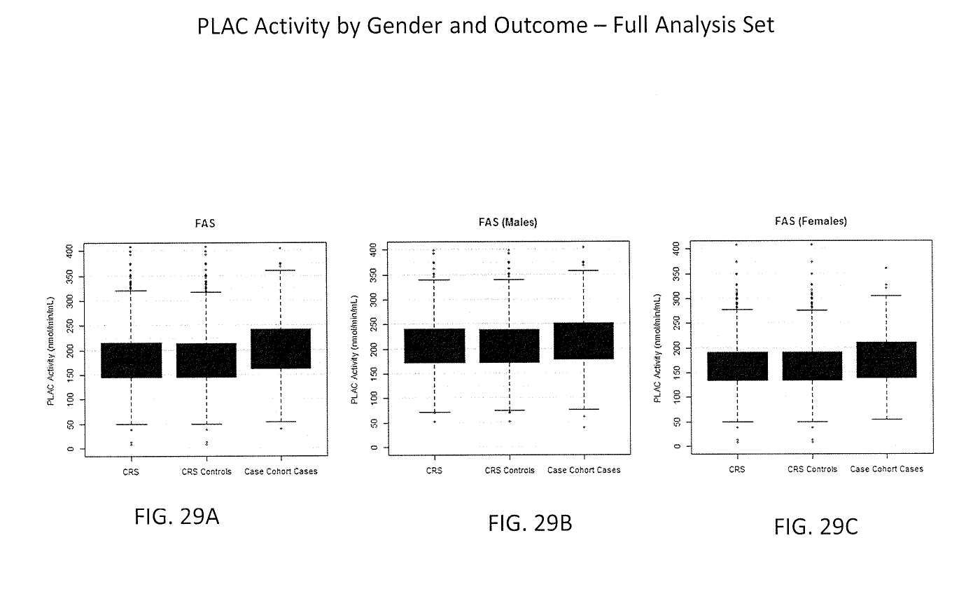

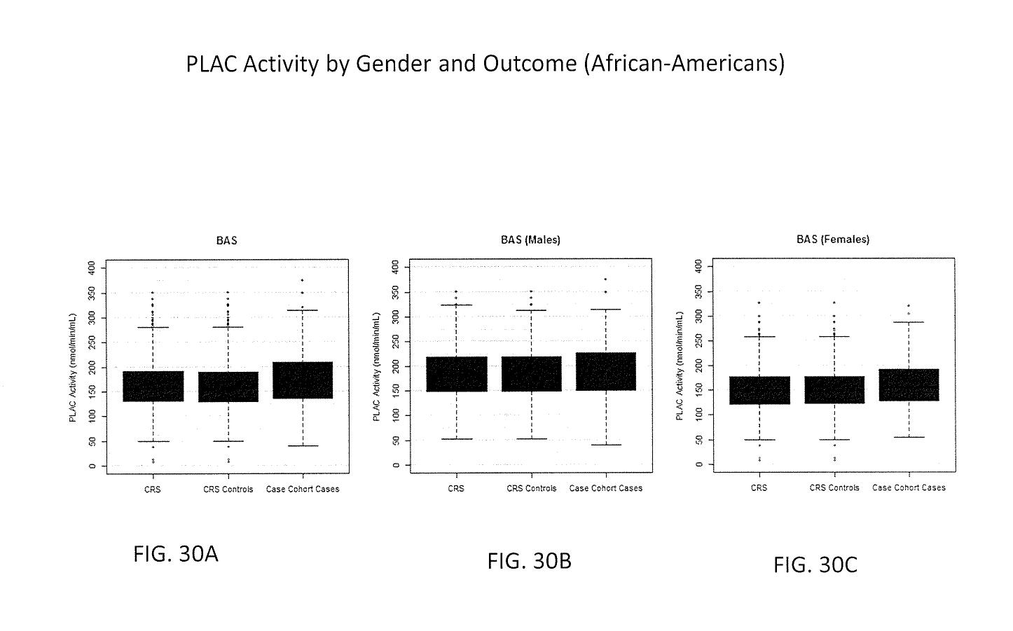

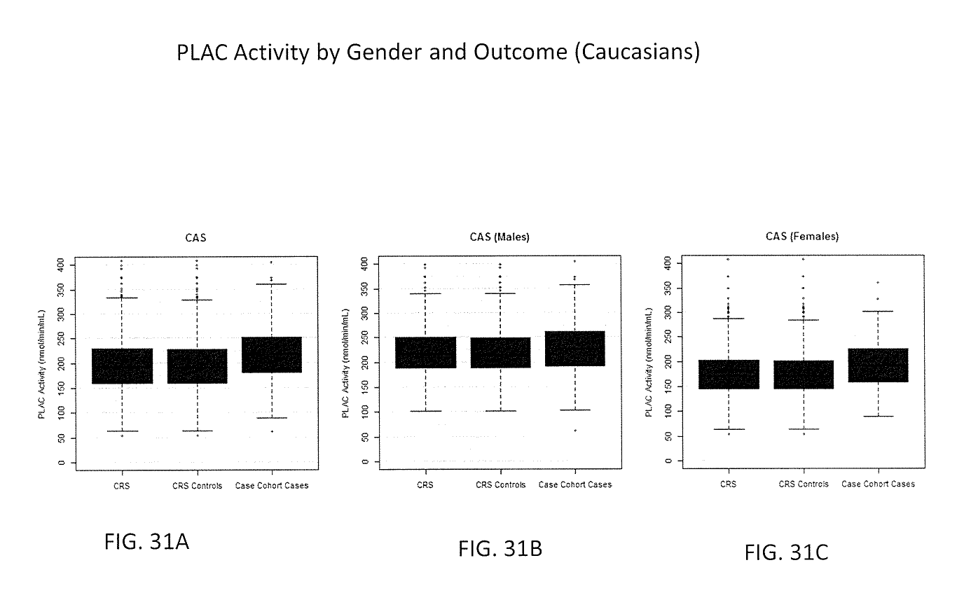

PLAC Activity distributions are also presented graphically by race for each gender and outcome stratum in FIGS. 29, 30 and 31. FIGS. 29A, 29B and 29C show LpPLA2 value distributions for males and females in the FAS. FIGS. 30A, 30B and 30C show LpPLA2 value distributions for males and females in African-Americans. FIGS. 31A, 31B and 31C show LpPLA2 value distributions for males and females in Caucasians.

As previously noted, the direction of these PLAC Activity differences by each race and gender are broadly consistent with the differences in CHD event rates subsequently experienced in the Indicated REGARDS Population by such subgroup. African-Americans and Females have lower CHD event rates than Caucasians and Males in the parent Indicated REGARDS Population, as well as in several other public health studies (see FIG. 3).

Conclusions—PLAC Activity Value Distributions

PLAC Activity measured in the CRS Indicated REGARDS Population sample had a median value of 177.8 nmol/min/mL (IQR 145.0-215.5 nmol/min/mL).

The Analysis Cut point of 225 nmol/min/mL selected represents the 79.6% CRS population percentile in the Indicated REGARDS Population. The differing percentile positions of the Analysis Cut point in the various race and gender subgroups are broadly consistent with the subsequent rate of CHD events experienced by each such subgroup in the Clinical Validation Study.

The pre-specified PLAC Activity Analysis Cut point of ≧225 nmol/min/mL has been shown in the efficacy analyses to be a statistically significant predictor for CHD events in each of these race and gender subgroups.

### Example 3

### Reference Range Study

Overview

In order to place the results of the Clinical Validation Study in context, a group of cross-sectional reference range analyses were performed, using subjects recruited and enrolled independently of the REGARDS study. The overarching goal was to assemble a representative contemporary set of subjects (the “Normal Reference Range Study”) with characteristics that overlapped REGARDS, but also enriched for sufficient subjects outside of the REGARDS inclusion criteria of being solely from African-American (Black) and Caucasian (White) races.

The Normal Reference Range Study and its subgroups were first analyzed independently. Subsequently, the non-overlapping race subgroups of the Normal Reference Range Study, specifically Asians and Hispanics, were compared to the two REGARDS race subgroups of Blacks and Whites (the “Racial Diversity Study”).

Normal Reference Range Study

Study Summary.

The Normal Reference Range Study was recruited from multiple sites, with relatively few selection criteria. Its goal was to assemble a largely unselected cross-sectional study set covering the broadest range of age and race subgroups, targeting balanced genders, as partitioning and subgroup analysis of these categories was desired, rather than a homogenous population or a more rigid set of population inclusion and exclusion criteria. Additionally, it specifically enriched for subjects excluded from the REGARDS study design.

Enrollment Criteria and Subject Assessment.

The study consisted of samples from 1,108 apparently healthy subjects collected at three US sites during the period from January through December 2013. Two sites, recruiting 617 and 308 subjects, respectively, were outpatient laboratories on the West and East Coast (Nevada and South Carolina), providing remnant samples from an all-comer population aged 35 or older; these samples provided only self-reported race, age, and gender information. The third source, recruiting 183 subjects, was from a West Coast (California) tertiary care inpatient facility laboratory, which utilized a detailed self-reported questionnaire to exclude prevalent disease given the higher risk hospital intake population, and collected all ages from age 20; other than the exclusion criteria, these samples also included only race, age, and gender information.

Biochemical Sampling Information.

Venous blood samples were obtained at study enrollment, processed, and stored at −80° C. until time of the PLAC Activity measurement. Samples were shipped directly from the participating laboratories to Sponsor.

Other Methods. Analysis methods were adapted from CLSI document C28-A3, Defining, Establishing and Verifying Reference Intervals in the Clinical Laboratory; Approved Guideline-Third Edition, 2010 (CLSI 2010). Given the large number of subjects tested, all reference range analysis was performed without removal of outliers and the recommended non-parametric methods used for reference interval and percentile calculations.

Clinical Characteristics.

The 1,108 subjects were 46.3% male and 53.7% female, with a median age of 57 years (mean 56.4, SD 15.8). A broad racial composition was achieved, at 19.9% Black (African-American), 25.7% Asian, 18.0% Hispanic, and 36.5% White (Caucasian).

PLAC Activity Values by Subgroup.

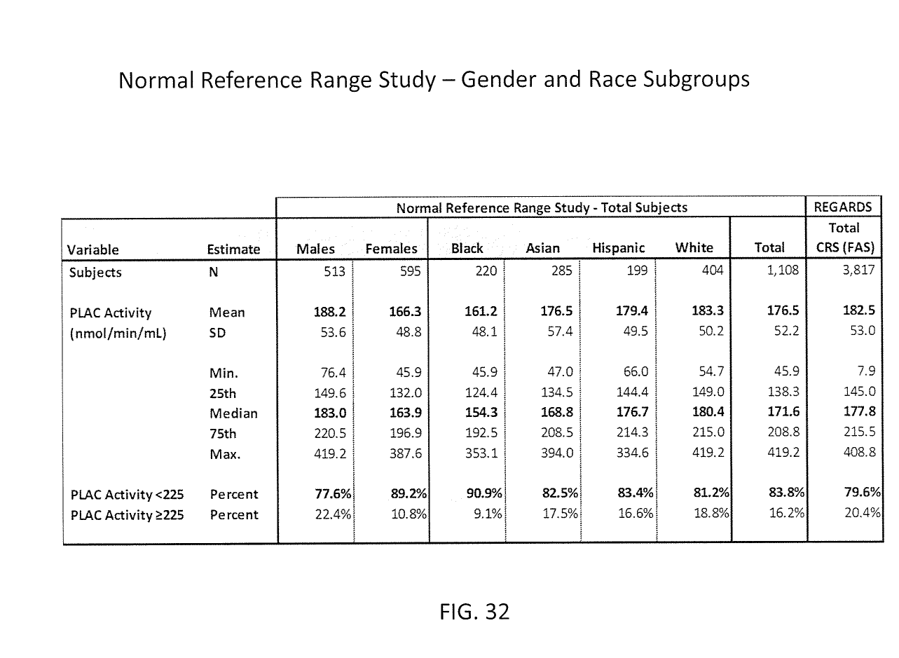

PLAC Activity testing results are summarized in FIG. 32 by the gender and race subgroups of interest, providing reference intervals for each together with the total Normal Reference Range Study. The same methods (mean, standard deviation, inter-quartile range (IQR), median, min and max) were used as in previous PLAC Activity value distribution tables, with the percentile position of the Analysis Cut point provided within each such subgroup. The Clinical Validation Study values for the total CRS of the Indicated REGARDS Population (FAS) are also provided for convenient comparison.

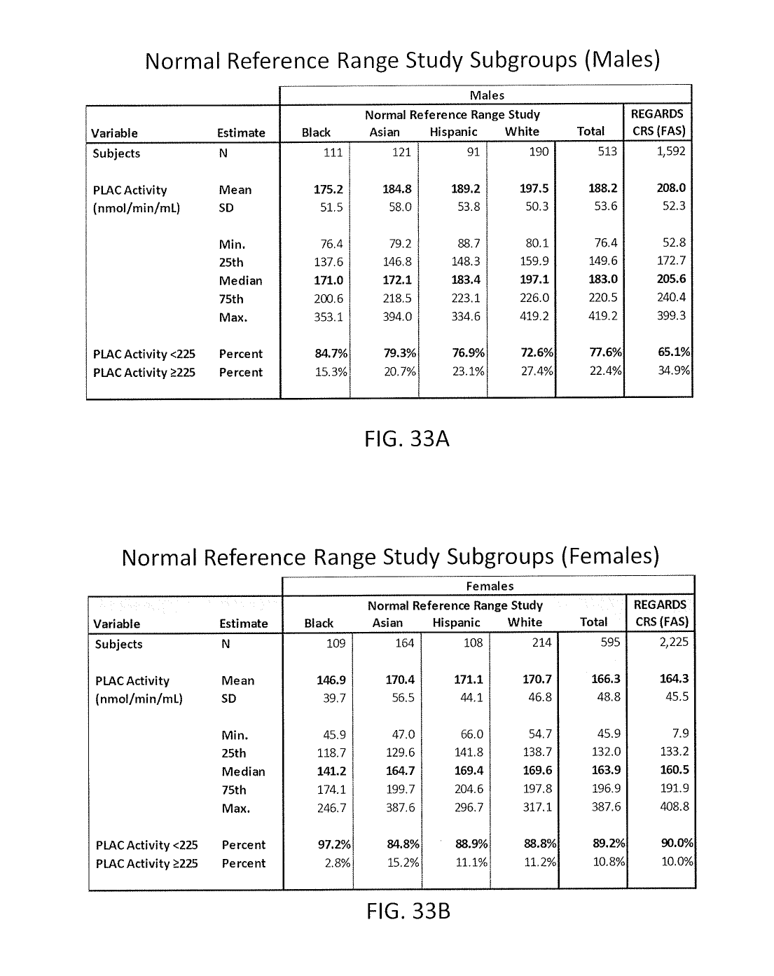

The analysis summarized in FIG. 32 is also presented for each individual gender in FIGS. 33A (males) and 33B (females) (Normal Reference Range Study Subgroups by Gender).

Despite the very different study designs, overall PLAC Activity value distributions are quite comparable between the total subjects of the Normal Reference Range Study and the total CRS, with median PLAC Activity values of 171.6 (IQR 138.3-208.8) and 177.8 (IQR 145.0-215.5) nmol/min/mL, respectively. The percentile position of the Analysis Cut point was the 83.8^{th} percentile in the overall Normal Reference Range Study versus the 79.6^{th} percentile seen in the CRS.

The PLAC Activity value distributions of each of the subgroups showed substantial overlap with the other subgroups, both within and between the two studies. For example, the interquartile range of males (the highest PLAC Activity value subgroup; IQR 149.6-220.5 nmol/min/mL) encompassed 73% of the interquartile range of females (the lowest PLAC Activity value subgroup; IQR 132.0-196.9 nmol/min/mL).

The same observations of lower female and black values seen in the Clinical Validation Study were confirmed in the Normal Reference Range Study. The Normal Reference Range Study additionally indicated that Asian and Hispanic populations have median PLAC Value distributions between those of Black (African-American) and White (Caucasian) populations.

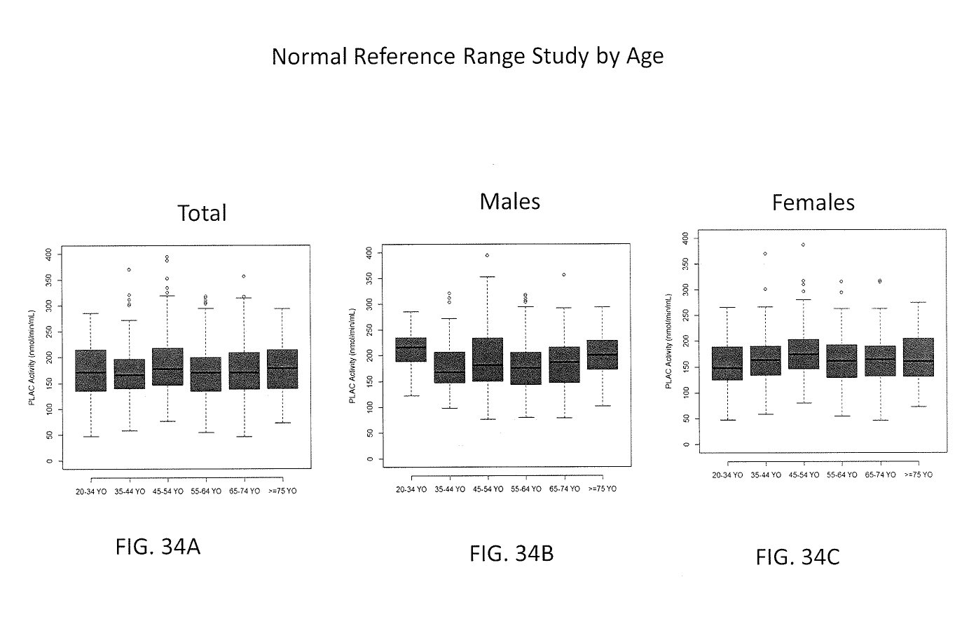

Following CLSI recommendations, the relationship of PLAC Activity and age was also explored, with similar results to those found in the Clinical Validation Study CRS. PLAC Activity value was not statistically correlated with age in the full study (R² of <0.001, p=0.518). PLAC Activity exhibited very consistent population value distributions across age decade categories, in both the full Normal Reference Study and within its individual genders. These analyses are summarized graphically in FIGS. 34A, 34B, 34C (Normal Reference Range Study Subgroups by Age).

Racial Diversity Study

The REGARDS study from which the Clinical Validation Study is selected limited its enrollment to African-Americans (Blacks) and Caucasians (Whites). These two racial groups constitute the vast majority of the United States population; in the 2010 US Census, individuals identifying themselves as White alone or Black alone represented 85.0% of the US population; when those identifying themselves as being White or Black in combination with other races are included, this proportion expands to 88.4% of the total US population (Humes et al. 2011).

The Racial Diversity Study was a cross-sectional analysis assembled of the selected race subpopulations from the Normal Reference Study which were not present in the Clinical Validation Study, specifically Asian and Hispanic subjects, who were then compared to Black and White subjects from the Clinical Validation Study Cohort Random Sample (as the direct sample of parent Indicated REGARDS Population).

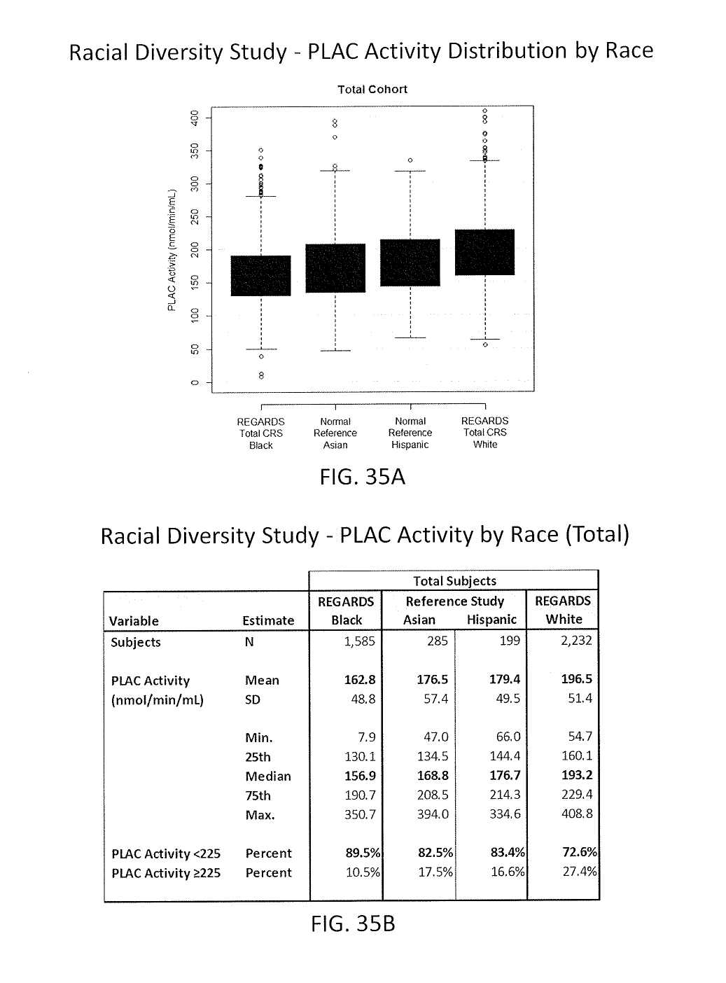

PLAC Activity testing results in the Racial Diversity Study are graphically presented in FIG. 35A. PLAC Activity testing results are also tabulated in FIG. 35B by the gender and race subgroups of interest, providing reference intervals for each together with the total Normal Reference Range Study. The same methods (mean, standard deviation, inter-quartile range (IQR), median, min and max) were used as in previous PLAC Activity value distribution tables, with the percentile position of the Analysis Cut point also provided within each such subgroup.

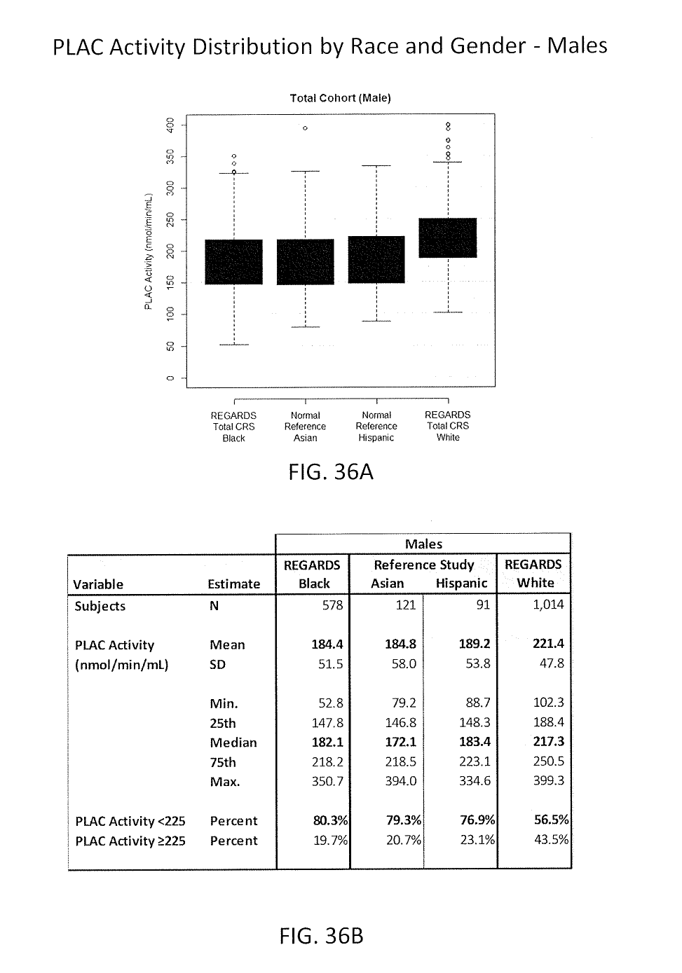

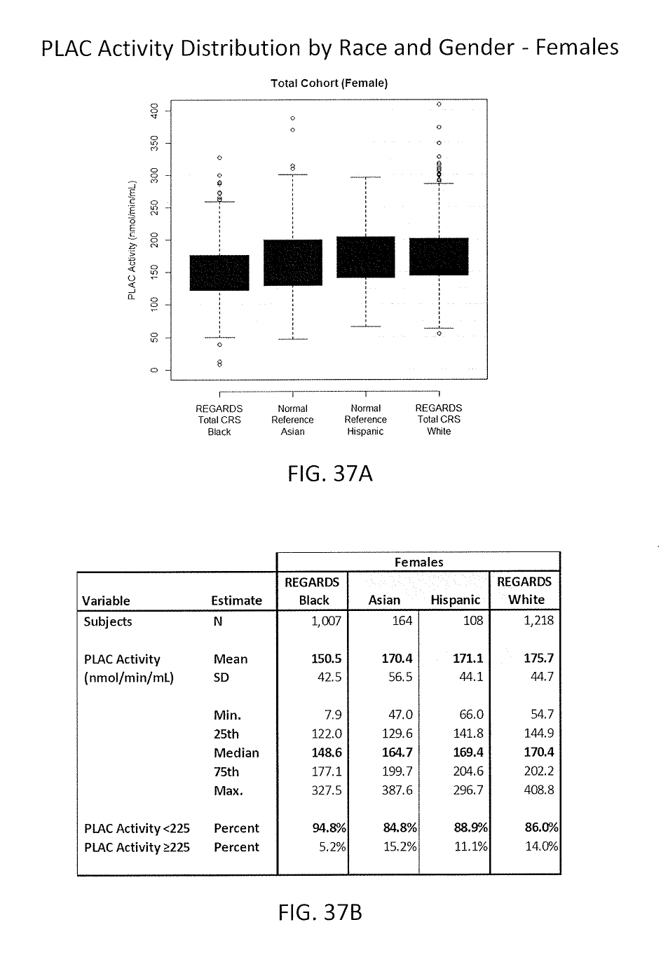

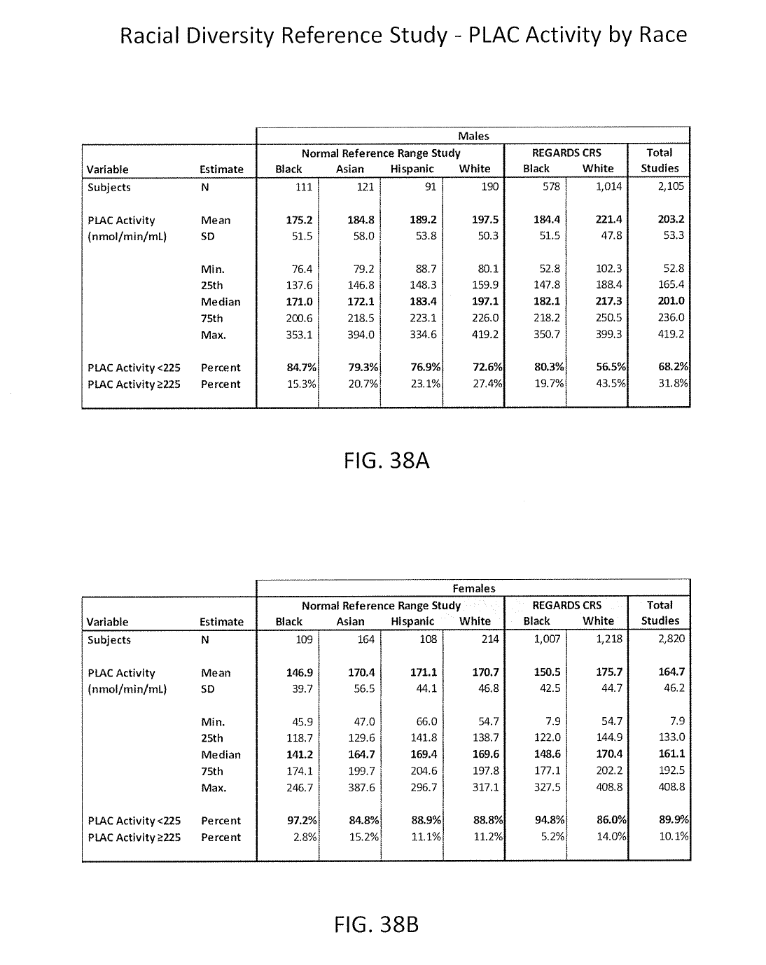

The above analyses are also presented for each individual gender in FIGS. 36 (males), 37 (females), and 38 (gender by race) (Racial Diversity Study Subgroups by Gender).

The Racial Diversity Study indicated that the PLAC Activity value distributions of the REGARDS Black and White populations from the Clinical Validation Study effectively include and surround those of Asian and Hispanic populations from the Normal Reference Study. The Clinical Validation Study races from REGARDS also include the minimum and maximum PLAC Activity values within the total Racial Diversity Study.

The PLAC Activity value distributions of each of the race subgroups in the Racial Diversity Study showed substantial overlap with the other remaining races. For example, the interquartile range of the REGARDS Whites (the highest PLAC Activity subgroup; IQR 160.1-229.4 nmol/min/mL) encompassed 65% of the interquartile range of Asians (the lowest non-REGARDS race subgroup; IQR 134.5-208.5 nmol/min/mL). Likewise, the interquartile range of the REGARDS Blacks (the lowest PLAC Activity subgroup; IQR 130.1-190.7 nmol/min/mL) encompassed 66% of the interquartile range of Hispanics (the highest non-REGARDS race subgroup; IQR 144.4-214.3 nmol/min/mL).

Discussion.

The Clinical Validation Study was shown to cover the entire range of PLAC Activity values expected in the remaining minority races that were not part of the original REGARDS study design. Furthermore, there has been no statistical interaction of race by PLAC Activity group even in the most divergent two races.

The Blacks and Whites in REGARDS cover the vast majority of the US population. Recent publications suggest that race is not an independent risk factor for coronary heart disease, and that rate differences by race are generally explained by the underlying prevalence of traditional cardiovascular risk factors (Safford et al. 2012). Race is not an input to any of the accepted cardiovascular risk calculators used in primary prevention (Goff et al. 2013). Work has also been published on the transferability of such risk factors and calculators across races (beyond those in which they are first trained and validated), indicating that such biomarkers remain valid and that racial differences in prediction can largely be recalibrated through simple adjustments based on the mean biomarker population values and expected incidence rates within the new race (D'Agostino et al. 2001). There is no evidence to suggest this would not be the case with PLAC Activity.

Conclusions—Reference Range Studies

The Normal Reference Range Study confirmed the PLAC Activity values seen in the Clinical Validation Study in a contemporary 2013 population.

Value distributions were very similar between the studies, despite the quite different study designs employed.

Interquartile ranges (IQRs) of all the subgroups substantially overlap both within and between the studies, with consistency in the relative values of genders and races.

Similar conclusions were reached on the percentile location of the analysis cut point, and the lack of PLAC Activity relationship with age.

The Racial Diversity Study indicated that the Black and White race subgroups within the Clinical Validation Study had the lowest and highest PLAC Activity distributions of the race subgroups.

Asian and Hispanic values were between those of Blacks and Whites.

### Example 4

### Clinical Program Conclusions

The presented studies above provide clinical evidence that the PLAC® Test for Lp-PLA₂ Activity, used in conjunction with clinical evaluation and patient risk assessment, is an aid in predicting risk of coronary heart disease (CHD) in patients with no prior history of cardiovascular events.

The following summarizes the primary conclusions of the Clinical Validation Study:

Primary Efficacy Analysis.

PLAC Activity values, using the pre-specified Analysis Cut point of ≧225 nmol/min/mL, were shown to be a statistically significant predictor for CUD events, classifying the Clinical Validation Study population into two PLAC Activity groups with statistically different absolute rates of CHD events.

The primary efficacy analysis achieved all pre-specified success criteria, with a Kaplan-Meier analysis log-rank p-value <0.001

At the five year median follow-up, the high PLAC Activity group had over twice the absolute rate for CHD events seen in the low PLAC Activity group with the following cumulative absolute rates of CHD events:

High PLAC Activity group 7.0% [6.2%-7.8%]

Low PLAC Activity group 3.3% [3.0%-3.6%]

The likelihood ratios and resulting post-test absolute event rates were demonstrated to represent a clinically important difference in absolute risk of CHD events for the high PLAC Activity group.

Positive Likelihood Ratio (LR+)=1.77 (High PLAC Activity group)

Negative Likelihood Ratio (LR−)=0.81 (Low PLAC Activity group)

Secondary Efficacy Analysis.

In the Clinical Validation Study, the high PLAC Activity group created by the Analysis Cut point of 225 nmol/min/mL was shown to have a statistically higher relative hazard for CHD events than that of the low PLAC Activity group of <225 nmol/min/mL.

The secondary efficacy analysis achieved all pre-specified success criteria, with a Cox proportional hazard ratio of 2.04 [95% CI 1.78-2.33, p<0.001].

Multivariate Cox Proportional Hazards Models.

Using Cox proportional hazards models, high PLAC Activity (defined by the Analysis Cut point) was shown to remain a statistically significant predictor of CHD events after full risk adjustment for other cardiovascular risk factors.

These factors included age, race, gender, smoking, hypertension, diabetes, LDL and HDL, treated as used in published guidelines.

After full risk-adjustment for these factors, the Cox hazard ratio of the high PLAC Activity group was 1.54 [95% CI 1.31-1.82, p<0.001] relative to the low PLAC Activity group.

Subgroup Analyses.

All Clinical Validation Study success criteria for the full study were also statistically significant within individual genders and races. There was no statistically significant interaction by either gender or by race.

Secondary Component Endpoints.

When analyzed independently, Myocardial Infarction, Coronary Revascularization, and CHD-Related Death (the Secondary Component Endpoints), achieved the same Clinical Validation Study success criteria as used in the main efficacy analyses of the Primary Clinical Endpoint.

PLAC Activity Tertile and Continuous Analyses.

Increases in PLAC Activity value were shown to be statistically significant predictors of the Primary Clinical Endpoint (total CHD composite) across PLAC Activity values other than the Analysis Cut point. Analyses were performed over PLAC Activity population tertile groups and also using continuous PLAC Activity values.

PLAC Activity Population Values and Analysis Cut Point.

PLAC Activity measured in the CRS Indicated REGARDS Population sample had a median value of 177.8 nmol/min/mL (IQR 145.0-215.5 nmol/min/mL).

The Analysis Cut point of 225 nmol/min/mL (used above) selected represents the 79.6% CRS population percentile.

Reference Range Studies Confirmed the PLAC Activity Values Seen in the Clinical Validation Study CRS in a Contemporary 2013 Population.

Value distributions were very similar between the studies, despite the quite different study designs employed.

Interquartile ranges (IQRs) of all the subgroups substantially overlap both within and between the studies, with consistency in the relative values of genders and races.

The Racial Diversity Study indicated that the Black and White race subgroups within the Clinical Validation Study, representing over 85% of the US population, had the lowest and highest PLAC Activity distributions of the race subgroups.

Asian and Hispanic values were between those of Blacks and Whites, and fully covered by the PLAC Activity value distribution within the CRS.

Clinical Program Summary

The Clinical Program results support the claim that the PLAC® Test for Lp-PLA₂ Activity, used in conjunction with clinical evaluation and patient risk assessment, is an aid in predicting risk of coronary heart disease (CHD) in patients with no prior history of cardiovascular events.

Normalization of the Cut Point

In general the robust cut point described above is described in terms of a PLAC Activity value of 225 nmol/min/mL. Although the cut point is expressed in terms of the PLAC Activity assay, it is apparent to one of skill in the art that the cut-point may refer to an amount equivalent to a PLAC Activity value of 225 nmol/min/mL; thus any Lp-PLA2 activity assay may be used and the cut point may be expressed in equivalent terms based on the specific parameters for that assay. For example, the activity may be expressed in activity units, or as a concentration of active Lp-PLA2. Note that the concentration of active Lp-PLA2 refers to the concentration of enzymatically active Lp-PLA2, which may be distinct from mass of Lp-PLA2. Further, there may be a relatively large variation when determining the active Lp-PLA2 based on the mass of the Lp-PLA2 present. For example, the cut point of 225 nmol/min/mL in an PLAC activity assay may be determined to have a mean of active Lp-PLA2 concentration of approximately 280 ng/ml (mean) with approximately 340 ng/ml (UCI) and 220 ng/ml (LCI).

In reference to other, including currently commercially available or later-developed Lp-PLA2 activity assays, a cut point for activity correlating to the clinical cut-point of 225 nmol/min/mL as validated for PLAC Test for Lp-PLA₂ activity in the clinical study for prediction of risk of coronary heart disease in subjects with no prior history of cardiovascular disease. Thus, for example, the cut-points for other activity assays may be readily determined by one of skill in the art by normalizing the assay to the PLAC Activity assay, e.g., using the calibrators present in the PLAC assay. One way to estimate the relative cut-points for other activity assays is to utilize linear regression statistics from method comparison studies to PLAC Test.

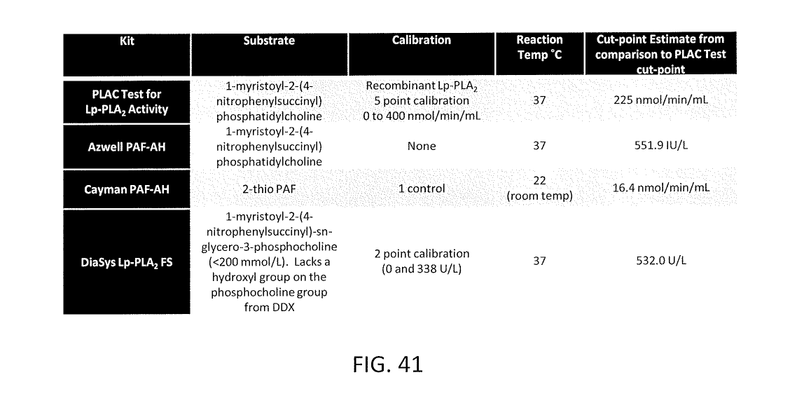

This method was used to generate the data shown in FIG. 41, illustrating the relative cut-points for other Lp-PLA₂ Activity kits, and calculating based on comparison plots of test results from a broad range ACP panel (n=40). Each assay/kit was operated using the multiple variables specified by each kit. All kits listed measure Lp-PLA₂ enzyme activity, however, under different conditions. Variations in substrates, calibration and reaction temperatures have impact on the final activity values, which may be in different units, as illustrated. Parameters for each kit are listed in the table of FIG. 41, with estimates of cut-points relative to PLAC Test for Lp-PLA₂ Activity shown in the far-right column.

In conclusion, the results of the studies herein have demonstrated for the first time that Lp-PLA2 activity levels can be used to assess risk of CHD events when compared to a cut point of an amount equivalent to a PLAC Activity value of 225 nmol/min/mL. Using a single cut point to determine risk is advantageous over previous methods without a cut point since the patient will be categorized at either low or high risk. For methods of evaluating Lp-PLA2 activity on a continuous risk scale (without a cut point) it may not be clear how to interpret a patient's Lp-PLA2 activity level (e.g., what constitutes an elevated level, and how high is too high). Further, the studies above have shown that a patient with Lp-PLA2 activity level equal to or above an amount equivalent to a PLAC Activity value of 225 nmol/min/mL are risk of a CHD event in as little as one year. These findings of short term risk due to Lp-PLA2 activity equal to or above an amount equivalent to a PLAC Activity value of 225 nmol/min/mL differ from previous findings that elevated Lp-PLA2 values, as a continuous variable, is associated with long term risk (e.g., 10 year risk, as in the Framingham Risk Score). Additionally, contrary to previous reports, these results demonstrate that it is not necessary to evaluate Lp-PLA2 activity values differently for different races. Put otherwise, patients with Lp-PLA2 activity level equal to or above an amount equivalent to a PLAC Activity value of 225 nmol/min/mL are risk of a CHD event, regardless of the patient's race. The results of these studies also demonstrate that women with an Lp-PLA2 activity level equal to or above an amount equivalent to a PLAC Activity value of 225 nmol/min/mL are at an increased risk of a CHD event compared to men. When using the newly identified cut point of an amount equivalent to a PLAC Activity value of 225 nmol/min/mL, Lp-PLA2 activity assays were capable of performing the methods described above.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method of treating a patient for coronary heart disease (CHD), the method comprising:
performing a test providing the amount of lipoprotein-associated phospholipase A2 (Lp-PLA2) activity in a patient sample, wherein the test comprises:
contacting a portion of the patient sample with an artificial substrate for Lp-PLA2 to enzymatically degrade the substrate,
detecting a signal that is proportional to the degradation of the artificial substrate for Lp-PLA2, and
determining an amount of Lp-PLA2 activity based on the signal;
determining that the amount of Lp-PLA2 activity is at or above a cut point when the amount of Lp-PLA2 activity is greater than or equal to a cut point comprising an amount equivalent to a measurement of Lp-PLA2 activity in serum or plasma value of 225 nmol/min/mL; and
treating the patient for CHD when the amount of Lp-PLA2 activity in the patient sample is at or above the cut point.

2. The method of claim 1, further comprising indicating that the amount of Lp-PLA2 activity is at or above the cut point when the amount of Lp-PLA2 activity in the patient sample is greater than or equal to an amount equivalent to the cut point.

3. The method of claim 1, wherein treating comprises administering an agent to treat CHD.

4. The method of claim 2, wherein administering comprises administering one or more medications selected from the group consisting of: an Lp-PLA2 inhibitor, an anti-inflammatory agent, an anti-thrombotic agent, an anti-platelet agent, a fibrinolytic agent, a lipid reducing agent, a direct thrombin inhibitor, a glycoprotein II b/IIIa receptor inhibitor, an agent that binds to cellular adhesion molecules and inhibits the ability of white blood cells to attach, an aldosterone antagonist, an angiotensin-converting enzyme (ACE) inhibitor, am angiotensin-receptor blocker (ARB), aspirin, a beta blocker, digoxin, a diuretic, an inotrope, digitalis, hydralazine, nitrates, statins, and warfarin.

5. (canceled)

6. The method of claim 1, wherein treating the patient for CHD comprises treating the patient of any race or gender when the amount of Lp-PLA2 activity in the patient sample is greater than or equal to an amount equivalent to an Lp-PLA2 activity of 225 nmol/min/mL.

7. A method of treating a patient for coronary heart disease (CHD), the method comprising:
performing a test providing the amount of Lp-PLA2 activity from a patient sample, wherein the test comprises:
contacting a portion of the patient sample with an artificial substrate for lipoprotein-associated phospholipase A2 (Lp-PLA2) to enzymatically degrade the substrate,
detecting a signal that is proportional to the degradation of the artificial substrate for Lp-PLA2, and
determining an amount of Lp-PLA2 activity based on the signal;
determining that the amount of Lp-PLA2 activity is at or above a cut point when the amount of Lp-PLA2 activity in the patient sample is greater than or equal to a cut point comprising an amount equivalent to a measurement of Lp-PLA2 activity in serum or plasma value of 225 nmol/min/mL; and
administering an agent to treat coronary heart disease when the amount of Lp-PLA2 activity in the patient sample is at or above the cut point.

8. The method of claim 7, further comprising indicating that the amount of Lp-PLA2 activity is at or above the cut point when the amount of Lp-PLA2 activity in the patient sample is greater than or equal to an amount equivalent to the cut point of 225 nmol/min/mL.

9. The method of claim 7, wherein administering comprises administering the agent to treat coronary heart disease when the amount of active Lp-PLA2 in the patient sample is greater than or equal to 220 ng/mL.

10. The method of claim 7, wherein administering comprises administering one or more medications selected from the group consisting of: an Lp-PLA2 inhibitor, an anti-inflammatory agent, an anti-thrombotic agent, an anti-platelet agent, a fibrinolytic agent, a lipid reducing agent, a direct thrombin inhibitor, a glycoprotein II b/IIIa receptor inhibitor, an agent that binds to cellular adhesion molecules and inhibits the ability of white blood cells to attach, an aldosterone antagonist, an angiotensin-converting enzyme (ACE) inhibitor, am angiotensin-receptor blocker (ARB), aspirin, a beta blocker, digoxin, a diuretic, an inotrope, digitalis, hydralazine, nitrates, statins, and warfarin.

11. The method of claim 7, wherein administering the agent comprises administering the agent to the patient, wherein the patient is of any race or gender when the amount of Lp-PLA2 activity in the patient sample is greater than or equal to an amount equivalent to 225 nmol/min/mL using a substrate of 1-myristoyl-2-(4-nitrophenyl succinyl) phosphatidylcholine.

12. A method of treating a patient for coronary heart disease (CHD), the method comprising:
contacting a portion of a sample from the patient with an artificial substrate for lipoprotein-associated phospholipase A2 (Lp-PLA2) comprising 1-myristoyl-2-(p-nitrophenylsuccinyl) phosphatidylcholine, to enzymatically degrade the substrate;
detecting a signal that is proportional to the degradation of the artificial substrate for Lp-PLA2;
calculating an amount of Lp-PLA2 activity based on the signal;
determining that the amount of Lp-PLA2 activity is at or above a cut point when the amount of Lp-PLA2 activity in the patient sample is greater than or equal to a cut point comprising an amount equivalent to a measurement of Lp-PLA2 activity in serum or plasma value of 225 nmol/min/mL; and
treating the patient for CHD when the amount of Lp-PLA2 activity in the patient sample is greater than or equal to the cut point.

13. The method of claim 12, further comprising indicating that the amount of Lp-PLA2 activity is at or above the cut point.

14. The method of claim 12, wherein treating comprises instructing a physician that the patient is at risk for coronary heart disease so that the physician may administer to the patient a therapy for CHD.

15. The method of claim 12, wherein treating comprises instructing a physician that the patient's Lp-PLA2 activity is greater than or equal to 225 nmol/min/mL and the patient is at risk for coronary heart disease so that the physician may administer to the patient a therapy for CHD.

16. The method of claim 12, wherein treating comprises administering an agent to treat CHD.

17. The method of claim 12, wherein treating comprises administering one or more medications selected from the group consisting of: an Lp-PLA2 inhibitor, an anti-inflammatory agent, an anti-thrombotic agent, an anti-platelet agent, a fibrinolytic agent, a lipid reducing agent, a direct thrombin inhibitor, a glycoprotein II b/IIIa receptor inhibitor, an agent that binds to cellular adhesion molecules and inhibits the ability of white blood cells to attach, an aldosterone antagonist, an angiotensin-converting enzyme (ACE) inhibitor, am angiotensin-receptor blocker (ARB), aspirin, a beta blocker, digoxin, a diuretic, an inotrope, digitalis, hydralazine, nitrates, statins, and warfarin.

18. A method of treating a patient of any race for coronary heart disease (CHD), the method comprising:
contacting a portion of a sample from the patient with an artificial substrate for lipoprotein-associated phospholipase A2 (Lp-PLA2) comprising 1-myristoyl-2-(p-nitrophenylsuccinyl) phosphatidylcholine, to enzymatically degrade the substrate;
detecting a signal that is proportional to the degradation of the artificial substrate for Lp-PLA2;
calculating an amount of Lp-PLA2 activity based on the signal;
determining that the amount of Lp-PLA2 activity is at or above a cut point when the amount of Lp-PLA2 activity in the patient sample is greater than or equal to a cut point comprising an amount equivalent to a measurement of Lp-PLA2 activity in serum or plasma value of 225 nmol/min/mL; and
administering an agent to the patient to treat coronary heart disease when the amount of Lp-PLA2 activity in the patient sample is at or above the cut point.

19. The method of claims 18, wherein administering comprising instructing a medical professional to administer an agent to the patient the patient to treat CHD.

20. The method of claim 18, wherein administering comprises administering one or more medications selected from the group consisting of: an Lp-PLA2 inhibitor, an anti-inflammatory agent, an anti-thrombotic agent, an anti-platelet agent, a fibrinolytic agent, a lipid reducing agent, a direct thrombin inhibitor, a glycoprotein II b/IIIa receptor inhibitor, an agent that binds to cellular adhesion molecules and inhibits the ability of white blood cells to attach, an aldosterone antagonist, an angiotensin-converting enzyme (ACE) inhibitor, am angiotensin-receptor blocker (ARB), aspirin, a beta blocker, digoxin, a diuretic, an inotrope, digitalis, hydralazine, nitrates, statins, and warfarin.
